(19) United States
(12) Patent Application Publication
(10) Pub. No.: US 2008/0221082 A1
(43) Pub. Date: Sep. 11, 2008

(54) Ligands of Integrin Receptors

(76) Inventors: Herve Geneste, Neuhofen (DE); Andreas Kling, Mannheim (DE); Udo Lange, Mannheim (DE); Werner Seitz, Plankstadt (DE); Claudia Isabella Graef, Mannheim (DE); Thomas Subkowski, Ladenburg (DE); Wilfried Hornberger, Neustadt (DE); Arnulf Lauterbach, Ludwigshafen (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/297,202

(22) PCT Filed: Jun. 6, 2001
(86) PCT No.: PCT/EP01/06397

§ 371 Date: Nov. 28, 2006

(30) Foreign Application Priority Data
Jun. 6, 2000 (DE) 100 27 514.1

(51) Int. Cl. A61K 31/55; C07D 487/14; C07D 235/30; C07D 277/02; A61K 31/426; A61K 31/4168

(52) U.S. Cl. 514/212.07; 540/523; 548/307.4; 548/190; 514/394; 514/370

(57) ABSTRACT

The invention relates to the use of cyclic compounds as ligands of integrin receptors, in particular as ligands of the α_{V}β₃ integrin receptor, the novel compounds themselves, their use, and pharmaceutical preparations comprising these compounds.

The present invention relates to the use of cyclic compounds as ligands of integrin receptors, in particular as ligands of the α_{V}β₃ integrin receptor, the novel compounds themselves, their use, and pharmaceutical preparations comprising these compounds.

Integrins are cell surface glycoprotein receptors which mediate interactions between similar and different cells as well as between cells and extracellular matrix proteins. They are involved in physiological processes, such as embryogenesis, hemostasis, wound healing, immune response and formation/maintenance of the tissue architecture.

Disturbances in the gene expression of cell adhesion molecules and functional disorders of the receptors can contribute to the pathogenesis of many disorders, such as tumors, thromboembolic events, cardiovascular disorders, lung diseases, disorders of the CNS, the kidney, the gastrointestinal tract or inflammation.

Integrins are heterodimers of an α- and a β-transmembrane subunit in each case, which are noncovalently bonded. Up to now, 16 different α- and 8 different β-subunits and 22 different combinations have been identified.

Integrin αᵥβ₃, also called the vitronectin receptor, mediates adhesion to a multiplicity of ligands—plasma proteins, extracellular matrix proteins, cell surface proteins, of which the majority contain the amino acid sequence RGD (Cell, 1986, 44, 517-518; Science 1987, 238, 491-497), such as vitronectin, fibrinogen, fibronectin, von Willebrand factor, thrombospondin, osteopontin, laminin, collagen, thrombin, tenascin, MMP-2, bone sialoprotein II, various viral, fungal, parasitic and bacterial proteins, natural integrin antagonists such as disintegrins, neurotoxins—mambin—and blood fluke proteins—decorsin, ornatin—and also some non-RGD ligands, such as Cyr-61 and PECAM-1 (L. Piali, J. Cell Biol. 1995, 130, 451-460; Buckley, J. Cell Science 1996, 109, 437-445, J. Biol. Chem. 1998, 273, 3090-3096).

A number of integrin receptors show cross-reactivity with ligands which contain the RGD motif. Thus integrin α_{IIb}β₃, also called the platelet fibrinogen receptor, recognizes fibronectin, vitronectin, thrombospondin, von Willebrand factor and fibrinogen.

Integrin αᵥβ₃ is expressed, inter alia, on endothelial cells, blood platelets, monocytes/macrophages, smooth muscle cells, some B cells, fibroblasts, osteoclasts and various tumor cells, such as melanoma, glioblastoma, lung, breast, prostate and bladder carcinomas, osteosarcomas or neuroblastomas.

Increased expression is observed under various pathological conditions, such as in the prothrombotic state, in vascular injury, tumor growth or metastasis or reperfusion and on activated cells, in particular on endothelial cells, smooth muscle cells or macrophages.

An involvement of integrin αᵥβ₃ has been demonstrated, inter alia, in the following syndromes:

cardiovascular disorders such as atherosclerosis, restenosis after vascular injury, and angioplasty (neointima formation, smooth muscle cell migration and proliferation) (J. Vasc. Surg. 1994, 19, 125-134; Circulation 1994, 90, 2203-2206),
acute kidney failure (Kidney Int. 1994, 46, 1050-1058; Proc. Natl. Acad. Sci. 1993, 90, 5700-5704; Kidney Int. 1995, 48, 1375-1385),
angiogenesis-associated microangiopathies such as diabetic retinopathy or rheumatoid arthritis (Ann. Rev. Physiol 1987, 49, 453-464; Int. Opthalmol. 1987, 11, 41-50; Cell 1994, 79, 1157-1164; J. Biol. Chem. 1992, 267, 1093.1-10934),
arterial thrombosis,
stroke (phase II studies with ReoPro, Centocor Inc., 8th annual European Stroke Meeting),
carcinomatous disorders, such as in tumor metastasis or in tumor growth (tumor-induced angiogenesis) (Cell 1991, 64, 327-336; Nature 1989, 339, 58-61; Science 1995, 270, 1500-1502),
osteoporosis (bone resorption after proliferation, chemotaxis and adhesion of osteoclasts to bone matrix) (FASEB J. 1993, 7, 1475-1482; Exp. Cell Res. 1991, 195, 368-375, Cell 1991, 64, 327-336),
high blood pressure (Am. J. Physiol. 1998, 275, H1449-H1454),
psoriasis (Am. J. Pathol. 1995, 147, 1661-1667),
hyperparathyroidism,
Paget's disease (J. Clin. Endocrinol. Metab. 1996, 81, 1810-1820),
malignant hypercalcemia (Cancer Res. 1998, 58, 1930-1935),
metastatic osteolytic lesions (Am. J. Pathol. 1997, 150, 1383-1393),
pathogenic protein (e.g. HIV-1 tat)-induced processes (e.g. angiogenesis, Kaposi's sarcoma) (Blood 1999, 94, 663-672)
inflammation (J. Allergy Clin. Immunol. 1998, 102, 376-381),
cardiac insufficiency, CHF, and also in
antiviral, antiparasitic, antifungal or antibacterial therapy and prophylaxis (adhesion and internalization) (J. Infect. Dis. 1999, 180, 156-166; J. Virology 1995, 69, 2664-2666; Cell 1993, 73, 309-319).

On account of its key role, pharmaceutical preparations which contain low-molecular weight integrin αᵥβ₃ ligands are of high therapeutic or diagnostic benefit, inter alia, in the indications mentioned.

Advantageous αᵥβ₃ integrin receptor ligands bind to the integrin αᵥβ₃ receptor with an increased affinity.

In contrast to integrin αᵥβ₃, particularly advantageous αᵥβ₃ integrin receptor ligands additionally have an increased selectivity and are less active with respect to the integrin α_{IIb}β₃ by at least a factor of 10, preferably at least a factor of 100.

For a multiplicity of compounds, such as anti-αᵥβ₃ monoclonal antibodies, peptides which contain the RGD binding sequence, natural, RGD-containing proteins (e.g. disintegrins) and low-molecular weight compounds, an integrin αᵥβ₃ antagonistic action has been shown and a positive in vivo effect demonstrated (FEBS Letts 1991, 291, 50-54; J. Biol. Chem. 1990, 265, 12267-12271; J. Biol. Chem. 1994, 269, 20233-20238; J. Cell Biol 1993, 51, 206-218; J. Biol. Chem. 1987, 262, 17703-17711; Bioorg. Med. Chem. 1998, 6, 1185-1208).

Antagonists of the α_{V}β₃ integrin receptor based on a bicyclic structural element are described in WO 9906049, WO 9905107, WO 9814192, WO 9724124, WO 9724122 and WO 9626190.

EP 540 334 and WO 9308174 describe bicyclic antagonists of the α_{IIb}β₃ integrin receptor.

WO 9407488 A1 describes compounds having a bicyclic molecular structure and which accelerate the release of growth hormone.

Further, vasopressin antagonists having a bicyclic molecular structure are described in the specifications EP 620216, WO 9534540, WO 9408582, WO 9802432, WO 9420473, JP 09221476 A1, JP 11060488 A1, WO 9404525, JP 04321669 A1, WO 9722591, as well as in Matsuhisa et al., Chem. Pharm. Bull. 1999, 47, 3, 329-339.

It is an object of the present invention to make available novel integrin receptor ligands having advantageous properties.

We have found that this object is achieved by the use of compounds of the formula I

B-G-L I
as ligands of integrin receptors,
where B, G and L have the following meanings:
L is a structural element of the formula I_{L}

—U-T I_{L}
where
T is a group COOH, a radical hydrolyzable to COOH or a radical bioisosteric to COOH and
—U— is —(X_{L})ₐ—(CR_{L}¹R_{L}²)_{b}—, —CR_{L}¹═CR_{L}²—, ethynylene or ═CR_{L}¹—, where
   a is 0 or 1,
   b is 0, 1 or 2
   X_{L} is CR_{L}³R_{L}⁴, NR_{L}⁵, oxygen or sulfur,
   R_{L}¹, R_{L}², R_{L}³, R_{L}⁴
      independently of one another are hydrogen, -T, —OH, —NR_{L}⁶R_{L}⁷, —CO—NH₂, a halogen radical, a branched or unbranched, optionally substituted C₁-C₆-alkyl, C₂-C₆-alkenyl, C₂-C₆-alkynyl, C₃-C₇-cycloalkyl, —CO—NH(C₁-C₆-alkyl), —CO—N(C₁-C₆-alkyl)₂ or C₁-C₄-alkoxy radical, an optionally substituted radical C₁-C₂-alkylene-T, C₂-alkenylene-T or C₂-alkynylene-T, an optionally substituted aryl or arylalkyl radical or in each case independently of one another are two radicals R_{L}¹ and R_{L}² or R_{L}³ and R_{L}⁴, or optionally R_{L}¹ and R_{L}³ together are an optionally substituted 3- to 7-membered saturated or unsaturated carbocycle or heterocycle, which can contain up to three identical or different heteroatoms O, N, S,
   R_{L}⁵, R_{L}⁶, R_{L}⁷
      independently of one another are hydrogen, a branched or unbranched, optionally substituted C₁-C₆-alkyl, C₃-C₇-cycloalkyl, CO—O—C₁-C₆-alkyl, SO₂—C₁-C₆-alkyl or CO—C₁-C₆-alkyl radical or an optionally substituted CO—O-alkylenearyl, SO₂-aryl, CO-aryl, SO₂-alkylenearyl or CO-alkylenearyl radical,
G is a structural element of the formula I_{G}

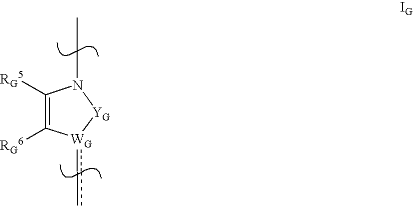

where
the structural element B is bonded to the structural element G via the ring nitrogen and the structural element L is bonded via W_{G},
Y_{G} is CO, CS, C═NR_{G}² or CR_{G}³R_{G}⁴,
R_{G}² is hydrogen, a hydroxyl group, a branched or unbranched, optionally substituted C₁-C₆-alkyl, C₁-C₄-alkoxy, C₃-C₇-cycloalkyl or —O—C₃-C₇-cycloalkyl radical or an optionally substituted aryl, —O-aryl, arylalkyl or —O-alkylenearyl radical,
R_{G}³, R_{G}⁴
   independently of one another are hydrogen or a branched or unbranched, optionally substituted C₁-C₆-alkyl, C₂-C₆-alkenyl, C₂-C₆-alkynyl or C₁-C₄-alkoxy radical or both radicals R_{G}³ and R_{G}⁴ together are a cyclic acetal —O—CH₂—CH₂—O— or —O—CH₂—O— or both radicals R_{G}³ and R_{G}⁴ together are an optionally substituted C₃-C₇-cycloalkyl radical,
R_{G}⁵ and R_{G}⁶
   independently of one another are hydrogen, a hydroxyl group, a branched or unbranched, optionally substituted C₁-C₆-alkyl or C₁-C₄-alkoxy radical, an optionally substituted aryl or arylalkyl radical or both radicals
R_{G}⁵ and R_{G}⁶ together are an optionally substituted, fused, unsaturated or aromatic 3- to 10-membered carbocycle or heterocycle, which can contain up to three different or identical heteroatoms O, N, S,
W_{G} is a structural element selected from the group of structural elements of the formulae I_{WG}¹ to I_{WG}⁴,

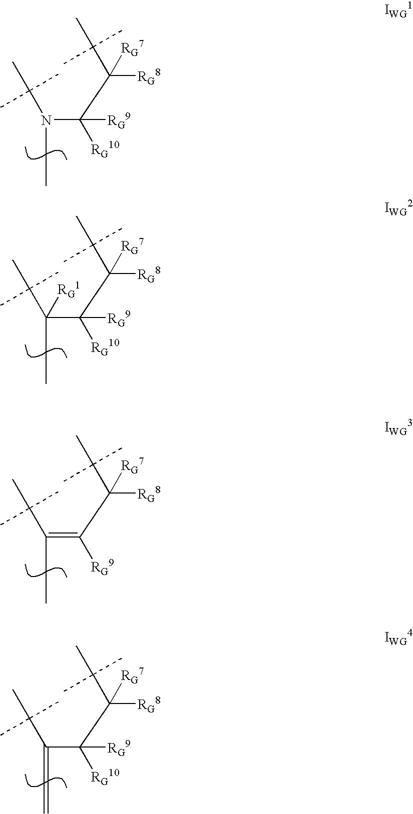

R_{G}¹ is hydrogen, halogen, a hydroxyl group or a branched or unbranched, optionally substituted C₁-C₆-alkyl or C₁-C₄-alkoxy radical,
R_{G}⁷, R_{G}⁸, R_{G}⁹, R_{G}¹⁰
   independently of one another are hydrogen, a hydroxyl group, —CN, halogen, a branched or unbranched, optionally substituted C₁-C₆-alkyl, C₂-C₆-alkenyl, C₂-C₆-alkynyl, C₁-C₄-alkylene-C₃-C₇-cycloalkyl, C₁-C₄-alkylene-C₃-C₇-heterocycloalkyl or C₁-C₄-alkylene-C₃-C₇-heterocycloalkenyl radical, a branched or unbranched, optionally substituted radical C₁-C₄-alkylene-OR_{G}¹¹, C₁-C₄-alkylene-CO—OR_{G}¹¹, C₁-C₄-alkylene-O—CO—R_{G}¹¹, C₁-C₄-alkylene-CO—R_{G}¹¹, C₁-C₄-alkylene-SO₂—NR_{G}¹²R_{G}¹³, C₁-C₄-alkylene-CO—NR_{G}¹²R_{G}¹³, C₁-C₄-alkylene-O—CO—NR_{G}¹²R_{G}¹³, C₁-C₄-alkylene-NR_{G}¹²R_{G}¹³ or C₁-C₄-alkylene-SR_{G}¹¹, C₁-C₄-alkylene-SO—R_{G}¹¹, a radical —S—R_{G}¹¹, —O—R_{G}¹¹, —SO—R_{G}¹¹, —SO₂—R_{G}¹¹, —CO—OR_{G}¹¹, —O—CO—R_{G}¹¹, —O—CO—NR_{G}¹²R_{G}¹³, —SO₂—NR_{G}¹²R_{G}¹³, —CO—NR_{G}¹²R_{G}¹³, —NR_{G}¹²R_{G}¹³ or CO—R_{G}¹¹, an optionally substituted C₃-C₇-cycloalkyl, C₃-C₇-heterocycloalkyl, C₃-C₇-heterocycloalkenyl, aryl, hetaryl, arylalkyl or hetarylalkyl radical or in each case independently of one another two radicals R_{G}⁷ and R_{G}⁹ or R_{G}⁸ and R_{G}¹⁰ or R_{G}⁷ and R_{G}⁸ or R_{G}⁹ and R_{G}¹⁰ together are an optionally substituted, saturated or unsaturated, nonaromatic, 3- to 7-membered carbocycle or heterocycle which can contain up to 3 heteroatoms selected from the group O, N, S and up to two double bonds,
R_{G}¹¹ is hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical,
R_{G}¹², R_{G}¹³
   independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NRG¹¹RG¹¹* or —CO—R_{G}¹¹, and
R_{G}¹¹* is a radical R_{G}¹¹ which is independent of R_{G}¹¹,
B is a structural element containing at least one atom which, under physiological conditions, as a hydrogen acceptor can form hydrogen bridges, where at least one hydrogen acceptor atom has a distance of 5 to 14 atomic bonds from structural element G along the shortest possible route along the structural element skeleton,
and the physiologically tolerable salts, prodrugs and the enantiomerically pure or diastereomerically pure and tautomeric forms.

In the structural element L, T is understood as meaning a group COOH, a radical hydrolyzable to COOH or a radical bioisosteric to COOH.

A radical hydrolyzable to COOH is understood as meaning a radical which changes into a group COOH after hydrolysis.

A group which may be mentioned by way of example as a radical T hydrolyzable to COOH is

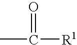

in which R¹ has the following meanings:
a) OM, where M can be a metal cation, such as an alkali metal cation, such as lithium, sodium, potassium, the equivalent of an alkaline earth metal cation, such as calcium, magnesium and barium, or an environmentally tolerable organic ammonium ion such as primary, secondary, tertiary or quaternary C₁-C₄-alkylammonium or ammonium ion, such as ONa, OK or OLi,
b) a branched or unbranched, optionally halogen-substituted C₁-C₈-alkoxy radical, such as methoxy, ethoxy, propoxy, 1-methylethoxy, butoxy, 1-methylpropoxy, 2-methylpropoxy, 1,1-dimethylethoxy, in particular methoxy, ethoxy, 1-methylethoxy, pentoxy, hexoxy, heptoxy, octoxy, difluoromethoxy, trifluoromethoxy, chlorodifluoromethoxy, 1-fluoroethoxy, 2-fluoroethoxy, 2,2-difluoroethoxy, 1,1,2,2-tetrafluoroethoxy, 2,2,2-trifluoroethoxy, 2-chloro-1,1,2-trifluoroethoxy or pentafluoroethoxy
c) a branched or unbranched, optionally halogen-substituted C₁-C₄-alkylthio radical such as methylthio, ethylthio, propylthio, 1-methylethylthio, butylthio, 1-methylpropylthio, 2-methylpropylthio or 1,1-dimethylethylthio radical
d) an optionally substituted —O-alkylenearyl radical, such as —O-benzyl
e) R¹ is further a radical —(O)ₘ—N(R¹⁸)(R¹⁹), in which m is 0 or 1 and R¹⁸ and R¹⁹, which can be identical or different, have the following meanings:
   hydrogen,
   a branched or unbranched, optionally substituted
   C₁-C₆-alkyl radical, such as methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl or the corresponding substituted radicals, preferably methyl, ethyl, propyl, butyl or i-butyl,
   C₂-C₆-alkenyl radical, such as vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl and 1-ethyl-2-methyl-2-propenyl, in particular 2-propenyl, 2-butenyl, 3-methyl-2-butenyl or 3-methyl-2-pentenyl or the corresponding substituted radicals,
   C₂-C₆-alkynyl radical, such as ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl, preferably 2-propynyl, 2-butynyl, 1-methyl-2-propynyl or 1-methyl-2-butynyl or the corresponding substituted radicals,
   C₃-C₈-cycloalkyl, such as cyclopropyl, cyclobutyl, cyclopentyl, cyclohexyl und cycloheptyl, cyclooctyl or the corresponding substituted radicals,
   or a phenyl radical, optionally mono- or polysubstituted, for example mono- to trisubstituted, by halogen, nitro, cyano, C₁-C₄-alkyl, C₁-C₄-halogenoalkyl, C₁-C₄-alkoxy, C₁-C₄-halogenoalkoxy or C₁-C₄-alkylthio such as 2-fluorophenyl, 3-chlorophenyl, 4-bromophenyl, 2-methylphenyl, 3-nitrophenyl, 4-cyanophenyl, 2-trifluoromethylphenyl, 3-methoxyphenyl, 4-trifluoroethoxyphenyl, 2-methylthiophenyl, 2,4-dichlorophenyl, 2-methoxy-3-methylphenyl, 2,4-dimethoxyphenyl, 2-nitro-5-cyanophenyl, 2,6-difluorophenyl,
   or R¹⁸ and R¹⁹ together form an optionally substituted, e.g. C₁-C₄-alkyl-substituted, C₄-C₇-alkylene chain closed to give a cycle, which can contain a heteroatom selected from the group consisting of oxygen, sulfur and nitrogen, such as —(CH₂)₄—, —(CH₂)₅—, —(CH₂)₆—, —(CH₂)₇—, —(CH₂)₂—O—(CH₂)₂—, —CH₂—S—(CH₂)₃—, —(CH₂)₂—O—(CH₂)₃—, —NH—(CH₂)₃—, —CH₂—NH—(CH₂)₂—, —CH₂—CH═CH—CH₂—, —CH═CH—(CH₂)₃—, —CO— (CH₂)₂—CO— or —CO— (CH₂)₃—CO—.

A radical bioisosteric to COOH is understood as meaning radicals which can replace the function of a group COOH in active compounds by equivalent bond donor/acceptor capabilities or by equivalent charge distribution.

Radicals which may be mentioned by way of example as radicals bioisosteric to —COOH are those such as described in “The Practice of Medicinal Chemistry, Editor: C. G. Wermuth, Academic Press 1996, pages 125 and 216, in particular the radicals —P═O(OH)₂, —SO₃H, tetrazole or acylsulfonamides.

Preferred radicals T are —COOH, —CO—O—C₁-C₈-alkyl or —CO—O-benzyl.

The radical —U— in the structural element L is a spacer selected from the group —(X_{L})ₐ—(CR_{L}¹R_{L}²)_{b}—, —CR_{L}¹═CR_{L}²—, ethynylene or ═CR_{L}¹-. In the case of the radical ═CR_{L}¹—, the structural element L is linked to the structural element G via a double bond.

X_{L} is a radical CR_{L}³R_{L}⁴, NR_{L}⁵, oxygen or sulfur.

Preferred radicals —U— are the radicals —CR_{L}¹═CR_{L}²—, ethynylene or —(X_{L})ₐ—(CR_{L}¹R_{L}²)_{b}—, where X_{L} is preferably CR_{L}³R_{L}⁴ (a=0 or 1) or oxygen (a=1).

Particularly preferred radicals —U— are the radicals —(X_{L})ₐ—(CR_{L}¹R_{L}²)_{b}—, where X_{L} is preferably CR_{L}³R_{L}⁴ (a=0 or 1) or oxygen (a 1).

Under R_{L}¹¹, R_{L}², R_{L}³ or R_{L}⁴ in the structural element L, a halogen radical is understood as meaning, for example, F, Cl, Br or I, preferably F.

Under R_{L}¹, R_{L}², R_{L}³ or R_{L}⁴ in structural element L, a branched or unbranched C₁-C₆-alkyl radical is understood as meaning, for example, methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl, preferably branched or unbranched C₁-C₄-alkyl radicals such as methyl, ethyl, propyl, 1-methylethyl, butyl, 1-methylpropyl, 2-methylpropyl or 1,1-dimethylethyl, particularly preferably methyl.

Under R_{L}¹, R_{L}², R_{L}³ or R_{L}⁴ in structural element L, a branched or unbranched C₂-C₆-alkenyl radical is understood as meaning, for example, vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl and 1-ethyl-2-methyl-2-propenyl, in particular 2-propenyl, 2-butenyl, 3-methyl-2-butenyl or 3-methyl-2-pentenyl.

Under R_{L}¹, R_{L}², R_{L}³ or R_{L}⁴ in structural element L, a branched or unbranched C₂-C₆-alkynyl radical is understood as meaning, for example, ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl, preferably ethynyl, 2-propynyl, 2-butynyl, 1-methyl-2-propynyl or 1-methyl-2-butynyl.

Under R_{L}¹, R_{L}², R_{L}³ or R_{L}⁴ in structural element L, a branched or unbranched C₃-C₇-cycloalkyl radical is understood as meaning, for example, cyclopropyl, cyclobutyl, cyclopentyl, cyclohexyl or cycloheptyl.

Under R_{L}¹, R_{L}², R_{L}³ or R_{L}⁴ in structural element L, a branched or unbranched C₁-C₄-alkoxy radical is understood as meaning, for example, methoxy, ethoxy, propoxy, 1-methylethoxy, butoxy, 1-methylpropoxy, 2-methylpropoxy or 1,1-dimethylethoxy.

The radicals —CO—NH(C₁-C₆-alkyl), —CO—N(C₁-C₆-alkyl)₂ are secondary or tertiary amides and are composed of the amide bond and the corresponding C₁-C₆-alkyl radicals such as described above for R_{L}¹, R_{L}², R_{L}³ or R_{L}⁴.

The radicals R_{L}¹, R_{L}², R_{L}³ or R_{L}⁴ can furthermore be a radical

C₁-C₂-alkylene-T, such as methylene-T or ethylene-T, C₂-alkenylene-T, such as ethenylene-T or C₂-alkynylene-T, such as ethynylene-T,
an aryl radical, such as phenyl, 1-naphthyl or 2-naphthyl or
an arylalkyl radical, such as benzyl or ethylenephenyl (homobenzyl),
where the radicals can optionally be substituted.

Furthermore, two radicals R_{L}¹ and R_{L}² or R_{L}³ and R_{L}⁴ or optionally R_{L}¹ and R_{L}³ can in each case independently of one another together be an optionally substituted 3- to 7-membered saturated or unsaturated carbocycle or heterocycle, which can contain up to three different or identical heteroatoms O, N, S.

All radicals for R_{L}¹, R_{L}², R_{L}³ or R_{L}⁴ can be optionally substituted. For the radicals R_{L}¹, R_{L}², R_{L}³ oder R_{L}⁴ and all further substituted radicals of the description below, suitable substituents, if the substituents are not specified in greater detail, are independently of one another up to 5 substituents, for example selected from the following group:

—NO₂, —NH₂, —OH, —CN, —COOH, —O—CH₂—COOH, halogen, a branched or unbranched, optionally substituted C₁-C₄-alkyl radical, such as methyl, CF₃, C₂F₅ or CH₂F, —CO—O—C₁-C₄-alkyl, C₃-C₆-cycloalkyl, C₁-C₄-alkoxy, C₁-C₄-thioalkyl, —NH—CO—O—C₁-C₄-alkyl, —O—CH₂—COO—C₁-C₄-alkyl, —NH—CO—C₁-C₄-alkyl, —CO—NH—C₁-C₄-alkyl, —NH—SO₂—C₁-C₄-alkyl, —SO₂—NH—C₁-C₄-alkyl, —N(C₁-C₄-alkyl)₂, —NH—C₁-C₄-alkyl, or —SO₂—C₁-C₄-alkyl radical, such as —SO₂—CF₃, an optionally substituted —NH—CO-aryl, —CO—NH-aryl, —NH—CO—O-aryl, —NH—CO—O-alkylenearyl, —NH—SO₂-aryl, —SO₂—NH-aryl, —CO—NH-benzyl, —NH—SO₂-benzyl or —SO₂—NH-benzyl radical, an optionally substituted radical —SO₂—NR²R³ or —CO—NR²R³ where the radicals R² and R³ independently of one another can have the meaning R_{L}⁵ as below or both radicals R² and R³ together can be a 3- to 6-membered, optionally substituted, saturated, unsaturated or aromatic heterocycle which, in addition to the ring nitrogen, can contain up to three further different or identical heteroatoms O, N, S, and optionally two radicals substituted on this heterocycle can together be a fused, saturated, unsaturated or aromatic carbocycle or heterocycle which can contain up to three different or identical heteroatoms O, N, S, and the cycle can be optionally substituted or a further, optionally substituted cycle can be fused to this cycle.

If not specified in greater detail, in all terminally bonded, substituted-hetaryl radicals of the description, two substituents can form a fused 5- to 7-membered, unsaturated or aromatic carbocycle.

Preferred radicals R_{L}¹, R_{L}², R_{L}³ or R_{L}⁴ are independently of one another hydrogen, halogen, a branched or unbranched, optionally substituted C₁-C₄-alkyl, C₁-C₄-alkoxy or C₃-C₇-cycloalkyl radical or the radical —NR_{L}⁶R_{L}⁷.

Particularly preferred radicals R_{L}¹, R_{L}², R_{L}³ or R_{L}⁴ are independently of one another hydrogen, fluorine or a branched or unbranched, optionally substituted C₁-C₄-alkyl radical, preferably methyl.

The radicals R_{L}⁵, R_{L}⁶, R_{L}⁷ in structural element L are independently of one another hydrogen, a branched or unbranched, optionally substituted

C₁-C₆-alkyl radical, for example as described above for R_{L}¹,
C₃-C₇-cycloalkyl radical, for example as described above for R_{L}¹,
CO—O—C₁-C₆-alkyl, SO₂—C₁-C₆-alkyl or CO—C₁-C₆-alkyl radical, which is composed of the group CO—O, SO₂ and CO and, for example, of the C₁-C₆-alkyl radicals described above for R_{L}¹,
or an optionally substituted CO—O-alkylenearyl, SO₂-aryl, SO₂-alkylenearyl or CO-alkylenearyl radical, which is composed of the group CO—O, SO₂ and CO and, for example, of the aryl or arylalkyl radicals described above for R_{L}¹.

Preferred radicals for R_{L}⁶ in structural element L are hydrogen, a branched or unbranched, optionally substituted C₁-C₄-alkyl, CO—O—C₁-C₄-alkyl, CO—C₁-C₄-alkyl or SO₂—C₁-C₄-alkyl radical or an optionally substituted CO—O-benzyl, SO₂-aryl, SO₂-alkylenearyl or CO-aryl radical.

Preferred radicals for R_{L}⁷ in structural element L are hydrogen or a branched or unbranched, optionally substituted C₁-C₄-alkyl radical.

Preferred structural elements L are composed of the preferred radicals of the structural element.

Particularly preferred structural elements L are composed of the particularly preferred radicals of the structural element.

G is a structural element of the formula I_{G}

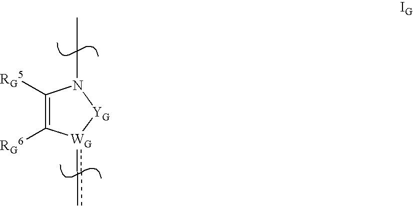

where the structural element B is bonded via the ring nitrogen and the structural element L is bonded via W_{G} to the structural element G, optionally via a double bond.

Y_{G} in structural element G is CO, CS, C═NR_{G}² or CR_{G}³R_{G}⁴, preferably CO, C═NR_{G}² or CR_{G}³R_{G}⁴, particularly preferably CO or CR_{G}³R_{G}⁴.

R_{G}² in structural element G is hydrogen, a hydroxyl group, a branched or unbranched, optionally substituted C₁-C₆-alkyl, C₁-C₄-alkoxy or C₃-C₇-cycloalkyl radical, for example as described above for R_{L}¹ in each case,

an optionally substituted —O—C₃-C₇-cycloalkyl radical, which is composed of an ether group and, for example, of the C₃-C₇-cycloalkyl radical described above for R_{L}¹,
an optionally substituted aryl or arylalkyl radical, for example as described above for R_{L}¹ in each case or
an optionally substituted —O-aryl or —O-alkylenearyl radical, which is composed of a group —O— and, for example, of the aryl or arylalkyl radicals described above for R_{L}¹.

Branched or unbranched, optionally substituted C₁-C₆-alkyl, C₂-C₆-alkenyl, C₂-C₆-alkynyl or C₁-C₄-alkoxy radicals for R_{G}³ or R_{G}⁴ in structural element G independently of one another are understood as meaning, for example, the corresponding radicals in each case described above for R_{L}¹.

Further, both radicals R_{G}³ and R_{G}⁴ can together form a cyclic acetal, such as —O—CH₂—CH₂—O— or —O—CH₂—O—.

Furthermore, both radicals R_{G}³ and R_{G}⁴ can together form an optionally substituted C₃-C₇-cycloalkyl radical.

Preferred radicals for R_{G}³ or R_{G}⁴ are independently of one another hydrogen, C₁-C₄-alkyl or C₁-C₄-alkoxy.

Branched or unbranched, optionally substituted C₁-C₆-alkyl or C₁-C₄-alkoxy radicals and optionally substituted aryl or arylalkyl radicals for R_{G}⁵ and R_{G}⁶ in structural element G independently of one another are, for example, the corresponding radicals in each case described above for R_{L}¹.

Further, both radicals R_{G}⁵ and R_{G}⁶ can together form an optionally substituted, fused, unsaturated or aromatic 3- to 10-membered carbocycle or heterocycle, which can contain up to three different or identical heteroatoms O, N, S.

Preferred radicals for R_{G}⁵ and R_{G}⁶ are independently of one another hydrogen or optionally substituted aryl radicals, preferably phenyl or arylalkyl radicals, preferably benzyl, and in each case both radicals R_{G}⁵ and R_{G}⁶ together can contain an optionally substituted, fused, unsaturated or aromatic 3- to 10-membered carbocycle or heterocycle which can contain up to three different or identical heteroatoms O, N, S.

In particularly preferred radicals for R_{G}⁵ and R_{G}⁶, both radicals R_{G}⁵ and R_{G}⁶ together form an optionally substituted, fused, unsaturated or aromatic 3- to 6-membered carbocycle or heterocycle, for example selected from one of the following doubly bonded structural formulae:

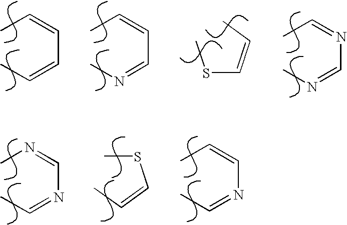

in particular selected from one of the following, doubly bonded structural formulae:

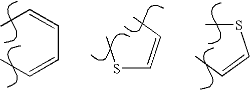

Suitable substituents of these fused, unsaturated or aromatic 3- to 10-membered carbocycles or heterocycles which together can form R_{G}⁵ and R_{G}⁶ are in particular substituents such as generally described above.

Particularly preferred substituents of these fused, unsaturated or aromatic 3- to 10-membered carbocycles or heterocycles which together can form R_{G}⁵ and R_{G}⁶ are independently of one another up to four substituents selected from the following group:

hydroxyl, —CN, F or Cl or a branched or unbranched, optionally substituted C₁-C₄-alkoxy or C₁-C₄-alkyl radical, such as methoxy, methyl, CF₃, C₂F₅ or CH₂F.

W_{G} is a structural element selected from the group of structural elements of the formulae I_{WG}¹ to I_{WG}⁴, where the dashed lines intersect the atomic bonds within the structural element G and the carbon atom substituted by R_{G}⁷ and R_{G}⁸ is bonded to Y_{G}.

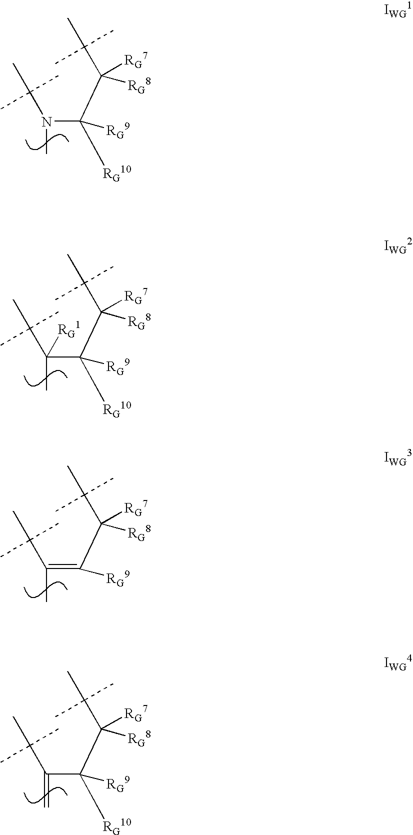

In a preferred embodiment, W_{G} is a structural element selected from the group of structural elements of the formulae I_{WG}² and I_{WG}³, in particular the structural element of the formula I_{WG}².

R_{G}¹ in structural element W_{G} is hydrogen, halogen, such as Cl, F, Br or I, a hydroxyl group or a branched or unbranched, optionally substituted C₁-C₆-alkyl radical, preferably C₁-C₄-alkyl or C₁-C₄-alkoxy radical such as in each case described above for R_{L}¹.

Particularly preferred radicals for R_{G}¹ are hydrogen, methoxy or hydroxyl.

R_{G}⁷, R_{G}⁸, R_{G}⁹ and R_{G}¹⁰ in structural element G are independently of one another hydrogen, a hydroxyl group, CN, halogen, such as F, Cl, Br, I, a branched or unbranched, optionally substituted

C₁-C₆-alkyl radical, such as optionally substituted methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl,
C₂-C₆-alkenyl radical, such as optionally substituted vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl or 1-ethyl-2-methyl-2-propenyl,
C₂-C₆-alkynyl radical, such as optionally substituted ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl or 1-ethyl-1-methyl-2-propynyl,
an optionally substituted
C₃-C₇-cycloalkyl radical, such as optionally substituted cyclopropyl, cyclobutyl, cyclopentyl, cyclohexyl or cycloheptyl,
C₃-C₇-heterocycloalkyl radical, such as optionally substituted aziridinyl, diaziridinyl, oxiranyl, oxaziridinyl, oxetanyl, thiiranyl, thietanyl, pyrrolidinyl, piperazinyl, morpholinyl, piperidinyl, tetrahydrofuranyl, tetrahydropyranyl, 1,4-dioxanyl, hexahydroazepinyl, oxepanyl, 1,2-oxathiolanyl or oxazolidinyl,
C₃-C₇-heterocycloalkenyl radical, such as optionally substituted azirinyl, diazirinyl, thiirenyl, thietyl, pyrrolinyls, oxazolinyls, azepinyl, oxepinyl, α-pyranyl, β-pyranyl, γ-pyranyl, dihydropyranyls, 2,5-dihydropyrrolinyl or 4,5-dihydrooxazolyl,
a branched or unbranched, optionally substituted C₁-C₄-alkylene-C₃-C₇-cycloalkyl radical, which is composed, for example, of branched or unbranched C₁-C₄-alkylene radicals such as methylene, ethylene, propylene, n-butylene, isobutylene or t-butylene and, for example, the abovementioned C₃-C₇-cycloalkyl radicals,
a branched or unbranched optionally substituted C₁-C₄-alkylene-C₃-C₇-heterocycloalkyl or C₁-C₄-alkylene-C₃-C₇-heterocycloalkenyl radical, which is composed of optionally substituted C₁-C₄-alkylene radicals, such as methylene, ethylene, propylene, n-butylene, isobutylene or t-butylene and, for example, the abovementioned C₃-C₇-heterocycloalkyl or C₃-C₇-heterocycloalkenyl radicals, the radicals being preferred which in the cyclic moiety contain one or two heteroatoms selected from the group consisting of N, O and S and up to two double bonds,
a branched or unbranched, optionally substituted radical C₁-C₄-alkylene-O—R_{G}¹¹, C₁-C₄-alkylene-CO—OR_{G}¹¹, C₁-C₄-alkylene-O—CO—R_{G}¹¹, C₁-C₄-alkylene-CO—R_{G}¹¹, C₁-C₄-alkylene-SO₂—NR_{G}¹²R_{G}¹³, C₁-C₄-alkylene-CO—NR_{G}¹²R_{G}¹³, C₁-C₄-alkylene-O—CO—NR_{G}¹²R_{G}¹³, C₁-C₄-alkylene-NR_{G}¹²R_{G}¹³, C₁-C₄-alkylene-SR_{G}¹¹ or C₁-C₄-alkylene-SO—R_{G}¹¹, which is composed of branched or unbranched, optionally substituted C₁-C₄-alkylene radicals, such as methylene, ethylene, propylene, n-butylene, isobutylene or t-butylene, the corresponding groups —O—, —CO—, —S—, —N and the terminal radicals R_{G}¹¹, R_{G}¹² and R_{G}¹³ described below,
an optionally substituted
aryl radical, preferably optionally substituted phenyl, 1-naphthyl or 2-naphthyl,
arylalkyl radical, preferably optionally substituted benzyl or ethylenephenyl (homobenzyl),
hetaryl radical, preferably optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl or their fused derivatives such as indazolyl, indolyl, benzothiophenyl, benzofuranyl, indolinyl, benzimidazolyl, benzothiazolyl, benzoxazolyl, quinolinyl or isoquinolinyl,
hetarylalkyl radical, preferably optionally substituted —CH₂-2-pyridyl, —CH₂-3-pyridyl, —CH₂-4-pyridyl, —CH₂-2-thienyl, —CH₂-3-thienyl, —CH₂-2-thiazolyl, —CH₂-4-thiazolyl, CH₂-5-thiazolyl, —CH₂—CH₂-2-pyridyl, —CH₂—CH₂-3-pyridyl, —CH₂—CH₂-4-pyridyl, —CH₂—CH₂-2-thienyl, —CH₂—CH₂-3-thienyl, —CH₂—CH₂-2-thiazolyl, —CH₂—CH₂-4-thiazolyl or —CH₂—CH₂-5-thiazolyl or
a radical —S—R_{G}¹¹, —O—R_{G}¹¹, —SO—R_{G}¹¹, —SO₂—R_{G}¹¹, —CO—OR_{G}¹¹, —O—CO—R_{G}¹¹, —O—CO—NR_{G}¹²R_{G}¹³, —SO₂—NR_{G}¹²R_{G}¹³, —CO—NR_{G}¹²R_{G}¹³, —NR_{G}¹²R_{G}¹³, CO—R_{G}¹¹.

Further, two radicals R_{G}⁷ and R_{G}⁹ or R_{G}⁸ and R_{G}¹⁰ or R_{G}⁷ and R_{G}⁸ or R_{G}⁹ and R_{G}¹⁰ can in each case independently of one another together form an optionally substituted, saturated or unsaturated, nonaromatic, 3- to 7-membered carbocycle or heterocycle which can contain up to 3 heteroatoms selected from the group consisting of O, N, S and up to two double bonds.

Preferred radicals for R_{G}⁷, R_{G}⁸, R_{G}⁹ and R_{G}¹⁰ in the structural element G are independently of one another hydrogen, a hydroxyl group, —CN, halogen, a branched or unbranched, optionally substituted C₁-C₆-alkyl, C₂-C₆-alkenyl, C₂-C₆-alkynyl, C₁-C₄-alkylene-C₃-C₇-cycloalkyl, C₁-C₄-alkylene-C₃-C₇-heterocycloalkyl or C₁-C₄-alkylene-C₃-C₇-heterocycloalkenyl radical, a branched or unbranched, optionally substituted radical C₁-C₄-alkylene-OR_{G}¹¹, C₁-C₄-alkylene-CO—OR_{G}¹¹, C₁-C₄-alkylene-O—CO—R_{G}¹¹, C₁-C₄-alkylene-CO—R_{G}¹¹, C₁-C₄-alkylene-SO₂—NR_{G}¹²R_{G}¹³, C₁-C₄-alkylene-CO—NR_{G}¹²R_{G}¹³, C₁-C₄-alkylene-O—CO—NR_{G}¹²R_{G}¹³, C₁-C₄-alkylene-NR_{G}¹²R_{G}¹³ or C₁-C₄-alkylene-SR_{G}¹¹, C₁-C₄-alkylene-SO—R_{G}¹¹, a radical —S—R_{G}¹¹, —O—R_{G}¹¹, —SO—R_{G}¹¹, SO₂—R_{G}¹¹, —CO—OR_{G}¹¹, —O—CO—R_{G}¹¹, —O—CO—NR_{G}¹²R_{G}¹³, —SO₂—NR_{G}¹²R_{G}¹³, —CO—NR_{G}¹²R_{G}¹³, —NR_{G}¹²R_{G}¹³ or CO—R_{G}¹¹, an optionally substituted C₃-C₇-cycloalkyl, C₃-C₇-heterocycloalkyl, C₃-C₇-heterocycloalkenyl, aryl, hetaryl, arylalkyl or hetarylalkyl radical, as described above in each case.

Particularly preferred radicals for R_{G}⁷, R_{G}⁸, R_{G}⁹ and R_{G}¹⁰ in the structural element G are independently of one another hydrogen, F or a branched or unbranched, optionally substituted C₁-C₄-alkyl radical, as described above.

A branched or unbranched, optionally substituted C₁-C₈-alkyl radical for R_{G}¹¹, R_{G}¹² and R_{G}¹³ is understood as meaning independently of one another, for example, the C₁-C₆-alkyl radicals mentioned above for R_{G}¹, plus the radicals heptyl and octyl.

Preferred substituents of the branched or unbranched, optionally substituted C₁-C₈-alkyl radicals for R_{G}¹¹, R_{G}¹² and R_{G}¹³ independently of one another are the radicals halogen, hydroxyl, C₁-C₄-alkoxy, —CN, —COOH and —CO—O—C₁-C₄-alkyl.

A branched or unbranched, optionally substituted C₂-C₆-alkenyl, C₂-C₆-alkynyl or C₁-C₄-alkylene-C₃-C₇-cycloalkyl radical, an optionally substituted C₃-C₇-cycloalkyl, aryl, arylalkyl, hetaryl or hetarylalkyl radical for R_{G}¹¹, R_{G}¹² and R_{G}¹³ independently of one another is understood as meaning, for example, the corresponding radicals mentioned above for R_{G}¹.

Preferred, branched or unbranched, optionally substituted —C₁-C₅-alkylene-C₁-C₄-alkoxy radicals for R_{G}¹¹, R_{G}¹² and R_{G}¹³ are independently of one another methoxymethylene, ethoxymethylene, t-butoxymethylene, methoxyethylene or ethoxyethylene.

Preferred, branched or unbranched, optionally substituted mono- and bis-alkylaminoalkylene or acylaminoalkylene radicals for R_{G}¹¹, R_{G}¹² and R_{G}¹³ are independently of one another branched or unbranched, optionally substituted radicals —C₁-C₄-alkylene-NH(C₁-C₄-alkyl), —C₁-C₄-alkylene-N(C₁-C₄-alkyl)₂ or —C₁-C₄-alkylene-NH—CO—C₁-C₄-alkyl.

Preferred optionally substituted heterocycloalkyl, heterocycloalkenyl, C₁-C₄-alkyleneheterocycloalkyl or C₁-C₄-alkyleneheterocycloalkenyl radicals for R_{G}¹¹, R_{G}¹² and R_{G}¹³ are independently of one another the C₃-C₇-heterocycloalkyl, C₃-C₇-heterocycloalkenyl, C₁-C₄-alkylene-C₃-C₇-heterocycloalkyl or C₁-C₄-alkylene-C₃-C₇-heterocycloalkenyl radicals described above for R_{G}¹.

Particularly preferred, optionally substituted heterocycloalkyl, heterocycloalkenyl, C₁-C₄-alkyleneheterocycloalkyl or C₁-C₄-alkyleneheterocycloalkenyl radicals for R_{G}¹¹, R_{G}¹² and R_{G}¹³ are independently of one another the C₃-C₇-heterocycloalkyl, C₃-C₇-heterocycloalkenyl, C₁-C₄-alkylene-C₃-C₇-heterocycloalkyl or C₁-C₄-alkylene-C₃-C₇-heterocycloalkenyl radicals described above for R_{G}¹, one or two heteroatoms selected from the group consisting of N, O and S and up to two double bonds being contained in the cyclic moiety.

Further, R_{G}¹² and R_{G}¹³ can independently of one another be a radical —SO₂—R_{G}¹¹, —CO—O—R_{G}¹¹, —CO—NR_{G}¹¹R_{G}¹¹* or —CO—R_{G}¹¹, R_{G}¹¹* being a radical R_{G}¹¹ which is independent of R_{G}¹¹.

Preferred structural elements G are composed of at least one preferred radical of the structural element G, while the remaining radicals are widely variable.

Particularly preferred structural elements G are composed of the preferred radicals of the structural element G.

Very particularly preferred structural elements G are composed of the particularly preferred radicals of the structural element G.

Structural element B is understood as meaning a structural element comprising at least one atom which under physiological conditions can form hydrogen bridges as a hydrogen acceptor, at least one hydrogen acceptor atom having a distance of 5 to 14 atomic bonds from structural element G along the shortest possible route along the structural element skeleton. The arrangement of the structural skeleton of structural element B is widely variable.

Suitable atoms which under physiological conditions can form hydrogen bridges as hydrogen acceptors are, for example, atoms having Lewis base properties, such as the heteroatoms nitrogen, oxygen or sulfur.

Physiological conditions is understood as meaning a pH which prevails at the site in a body at which the ligands interact with the receptors. In the present case, the physiological conditions have a pH of, for example, 5 to 9.

In a preferred embodiment, structural element B is a structural element of the formula I_{B}

A-E- I_{B}
where A and E have the following meanings:
A is a structural element selected from the group:
   a 4- to 8-membered monocyclic saturated, unsaturated or aromatic hydrocarbon which can contain up to 4 heteroatoms selected from the group O, N and S, where, in each case independently of one another, the optionally present ring nitrogen or the carbons can be substituted, with the proviso that at least one heteroatom selected from the group O, N and S is contained in the structural element A,
   or
   a 9- to 14-membered polycyclic, saturated, unsaturated or aromatic hydrocarbon which can contain up to 6 heteroatoms selected from the group N, O and S, where, in each case independently of one another, the optionally present ring nitrogen or the carbons can be substituted, with the proviso that at least one heteroatom selected from the group O, N and S is contained in the structural element A,
   a radical

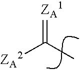

where
Z_{A}¹ is oxygen, sulfur or optionally substituted nitrogen and
Z_{A}² is optionally substituted nitrogen, oxygen or sulfur
and a radical

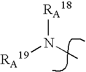

where
R_{A}¹⁸, R_{A}¹⁹
   independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R¹¹,
and
E is a spacer structural element which covalently bonds the structural element A to the structural element G, where the number of atomic bonds along the shortest possible route along the structural element skeleton E is 5 to 14.

In a particularly preferred embodiment, the structural element A is a structural element selected from the group of structural elements of the formulae I_{A}¹ to I_{A}¹⁸

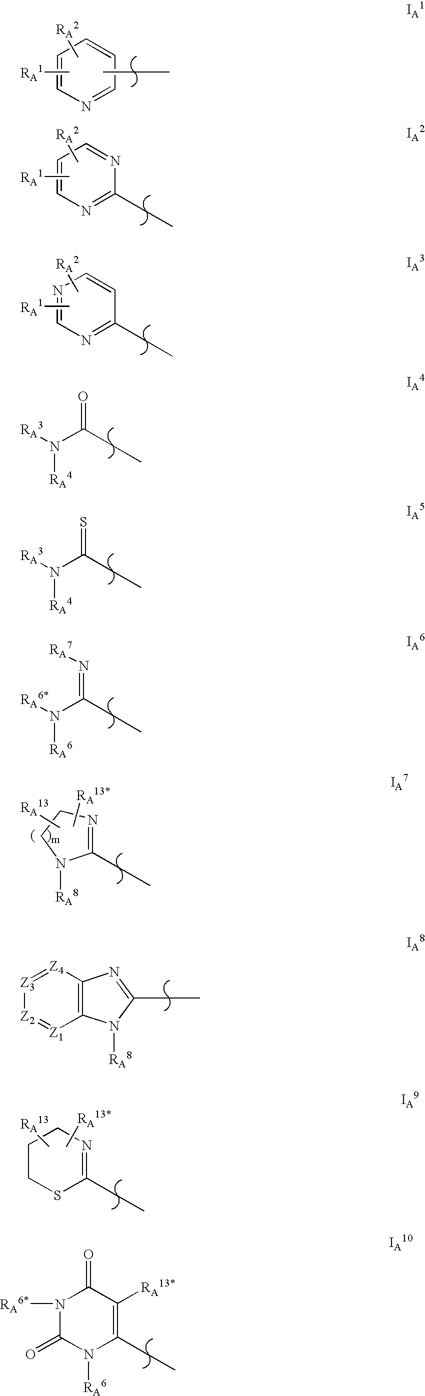

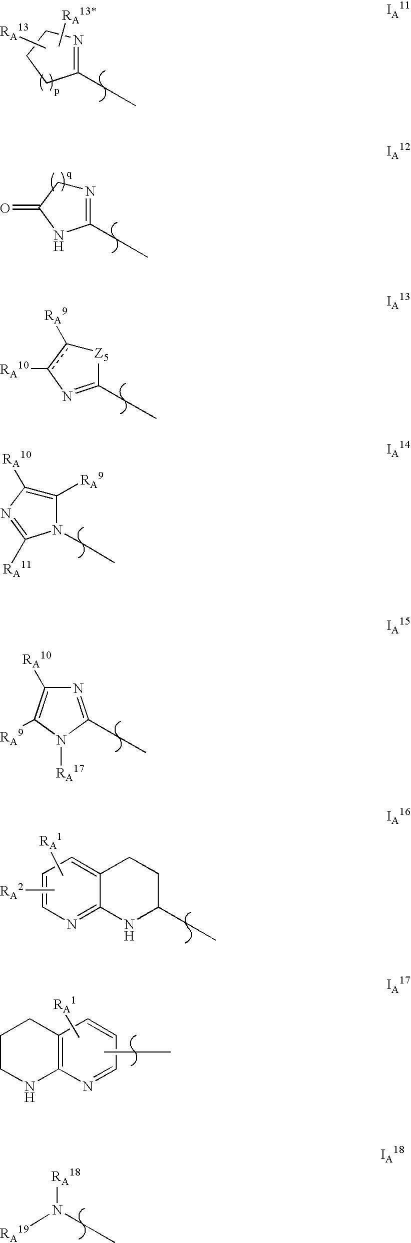

where
m, p, g
   independently of one another are 1, 2 or 3,
R_{A}¹, R_{A}²
   independently of one another are hydrogen, CN, halogen, a branched or unbranched, optionally substituted C₁-C₆-alkyl or CO—C₁-C₆-alkyl radical or an optionally substituted aryl, arylalkyl, hetaryl, hetarylalkyl or C₃-C₇-cycloalkyl radical or a radical CO—O—R_{A}¹⁴, O—R_{A}¹⁴, S—R_{A}¹⁴, NR_{A}¹⁵R_{A}¹⁶, CO—NR_{A}¹⁵R_{A}¹⁶ or SO₂NR_{A}¹⁵R_{A}¹⁶ or both radicals R_{A}¹ and R_{A}² together are a fused, optionally substituted, 5- or 6-membered, unsaturated or aromatic carbocycle or heterocycle which can contain up to three heteroatoms selected from the group O, N, and S,
R_{A}¹³, R_{A}¹³*
   independently of one another are hydrogen, CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,
   where
   R_{A}¹⁴ is hydrogen, a branched or unbranched, optionally substituted C₁-C₆-alkyl, alkylene-C₁-C₄-alkoxy, C₂-C₆-alkenyl, C₂-C₆-alkynyl or C₁-C₆-alkylene-C₃-C₇-cycloalkyl radical or an optionally substituted C₃-C₇-cycloalkyl, aryl, arylalkyl, hetaryl or hetarylalkyl radical,
   R_{A}¹⁵, R_{A}¹⁶,
      independently of one another are hydrogen, a branched or unbranched, optionally substituted C₁-C₆-alkyl, CO—C₁-C₆-alkyl, SO₂—C₁-C₆-alkyl, COO—C₁-C₆-alkyl, CO—NH—C₁-C₆-alkyl, arylalkyl, COO-alkylenearyl, SO₂-alkylenearyl, CO—NH-alkylenearyl, CO—NH-alkylenehetaryl or hetarylalkyl radical or an optionally substituted C₃-C₇-cycloalkyl, aryl, CO-aryl, CO—NH-aryl, SO₂-aryl, hetaryl, CO—NH-hetaryl or CO-hetaryl radical,
R_{A}³, R_{A}⁴
   independently of one another are hydrogen, —(CH₂)ₙ—(X_{A})ⱼ—R_{A}¹², or both radicals together are a 3- to 8-membered, saturated, unsaturated or aromatic N-heterocycle which can additionally contain two further, identical or different heteroatoms O, N or S, where the cycle is optionally substituted or a further, optionally substituted, saturated, unsaturated or aromatic cycle can be fused to this cycle,
   where
      n is 0, 1, 2 or 3,
      j is 0 or 1,
      X_{A} is —CO—, —CO—N(R_{X}¹)—, —N(R_{X}¹)—CO—, —N(R_{X}¹)—CO—N(R_{X}¹*)—, —N(R_{X}¹)—CO—O—, —O—, —S—, —SO₂—, —SO₂—N(R_{X}¹)—, —SO₂—O—, —CO—O—, —O—CO—, —O—CO—N(R_{X}¹), —N(R_{X}¹)— or —N(R_{X}¹)—SO₂—,
      R_{A}¹² is hydrogen, a branched or unbranched, optionally substituted C₁-C₆-alkyl radical, an optionally C₁-C₄-alkyl- or aryl-substituted C₂-C₆-alkynyl or C₂-C₆-alkenyl radical or a 3- to 6-membered, saturated or unsaturated heterocycle, substituted by up to three identical or different radicals, which can contain up to three different or identical heteroatoms O, N, S, a C₃-C₇-cycloalkyl, aryl or hetaryl radical, where two radicals together can be a fused, saturated, unsaturated or aromatic carbocycle or heterocycle which can contain up to three different or identical heteroatoms O, N, S, and the cycle can optionally be substituted or a further, optionally substituted, saturated, unsaturated or aromatic cycle can be fused to this cycle, or the radical R_{A}¹², together with R_{X}¹ or R_{X}¹* forms a saturated or unsaturated C₃-C₇-heterocycle which can optionally contain up to two further heteroatoms selected from the group O, S and N,
      R_{X}¹, R_{X}¹*
         independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl or SO₂—C₁-C₆-alkyl radical or an optionally substituted C₃-C₇-cycloalkyl, aryl, arylalkyl, CO—O-alkylenearyl, CO-alkylenearyl, CO-aryl, SO₂-aryl, hetaryl, CO-hetaryl or SO₂-alkylenearyl radical,
R_{A}⁶, R_{A}⁶*
   are hydrogen, a branched or unbranched, optionally substituted C₁-C₄-alkyl, —CO—O—C₁-C₄-alkyl, arylalkyl, —CO—O-alkylenearyl, —CO—O-allyl, —CO—C₁-C₄-alkyl, —CO-alkylenearyl, C₃-C₇-cycloalkyl or —CO-allyl radical or in the structural element I_{A}⁷ both radicals R_{A}⁶ and R_{A}⁶* together are an optionally substituted, saturated, unsaturated or aromatic heterocycle which, in addition to the ring nitrogen, can contain up to two further different or identical heteroatoms O, N, S,
R_{A}⁷ is hydrogen, —OH, —CN, —CONH₂, a branched or unbranched, optionally substituted
   C₁-C₄-alkyl, C₁-C₄-alkoxy, C₃-C₇-cycloalkyl or —O—CO—C₁-C₄-alkyl radical, or an optionally substituted arylalkyl, —O-alkylenearyl, —O—CO-aryl, —O—CO-alkylenearyl or —O—CO-allyl radical, or both radicals R_{A}⁶ and R_{A}⁷ together are an optionally substituted, unsaturated or aromatic heterocycle which, in addition to the ring nitrogen, can contain up to two further different or identical heteroatoms O, N, S,
R_{A}⁸ is hydrogen, a branched or unbranched, optionally substituted C₁-C₄-alkyl, CO—C₁-C₄-alkyl, SO₂—C₁-C₄-alkyl or CO—O—C₁-C₄-alkyl radical or an optionally substituted aryl, CO-aryl, SO₂-aryl, CO—O-aryl, CO-alkylenearyl, SO₂-alkylenearyl, CO—O-alkylenearyl or alkylenearyl radical,
R_{A}⁹, R_{A}¹⁰
   independently of one another are hydrogen, —CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶, or both radicals R_{A}⁹ and R_{A}¹⁰ together in the structural element I_{A}¹⁴ are a 5- to 7-membered saturated, unsaturated or aromatic carbocycle or heterocycle which can contain up to three different or identical heteroatoms O, N, S and is optionally substituted by up to three identical or different radicals,
R_{A}¹¹ is hydrogen, —CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,
R_{A}¹⁷ is hydrogen or, in the structural element I_{A}¹⁶, both radicals R_{A}⁹ and R_{A}¹⁷ together are a 5- to 7-membered saturated, unsaturated or aromatic heterocycle which, in addition to the ring nitrogen, can contain up to three different or identical heteroatoms O, N, S and is optionally substituted by up to three identical or different radicals,
R_{A}¹⁸, R_{A}¹⁹
   independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹¹ which is independent of R_{G}¹¹,
Z¹, Z², Z³, Z⁴
   independently of one another are nitrogen, C—H, C-halogen or a branched or unbranched, optionally substituted C—C₁-C₄-alkyl or C—C₁-C₄-alkoxy radical,
Z⁵ is NR_{A}⁸, oxygen or sulfur.

In a further very particularly preferred embodiment, the structural element A is a structural element of the formula I_{A}¹, I_{A}⁴, I_{A}⁷, I_{A}⁸ or I_{A}⁹.

For R_{A}¹ or R_{A}² independently of one another a branched or unbranched, optionally substituted C₁-C₆-alkyl radical is understood as meaning, for example, the corresponding radicals described above for R_{G}¹, preferably methyl or trifluoromethyl.

For R_{A}¹ or R_{A}² in the structural elements I_{A}¹, I_{A}², I_{A}³ or I_{A}¹⁷, the branched or unbranched, optionally substituted radical CO—C₁-C₆-alkyl is composed, for example, of the group CO and the branched or unbranched, optionally substituted C₁-C₆-alkyl radicals described above for R_{A}¹ or R_{A}².

Optionally substituted hetaryl, hetarylalkyl, aryl, arylalkyl or C₃-C₇-cycloalkyl radicals for R_{A}¹ or R_{A}² independently of one another are understood as meaning, for example, the corresponding radicals described above for R_{G}⁷.

For R_{A}¹ or R_{A}², the optionally substituted radicals CO—O—R_{A}¹⁴, O—R_{A}¹⁴, S—R_{A}¹⁴, NR_{A}¹⁵R_{A}¹⁶, CO—NR_{A}¹⁵R_{A}¹⁶ or SO₂NR_{A}¹⁵R_{A}¹⁶ are composed, for example, of the groups CO—O, O, S, N, CO—N or SO₂—N and the radicals R_{A}¹⁴, R_{A}¹⁵ or R_{A}¹⁶ described in greater detail below.

Further, both radicals R_{A}¹ and R_{A}² can together form a fused, optionally substituted, 5- or 6-membered, unsaturated or aromatic carbocycle or heterocycle which can contain up to three heteroatoms selected from the group consisting of O, N and S.

R_{A}¹³ and R_{A}¹³* are independently of one another hydrogen, CN,

halogen, such as fluorine, chlorine, bromine or iodine,
a branched or unbranched, optionally substituted C₁-C₆-alkyl radical, such as described above for R_{G}¹, preferably methyl or trifluoromethyl or
an optionally substituted aryl, arylalkyl, hetaryl or C₃-C₇-cycloalkyl radical or a radical CO—O—R_{A}¹⁴, O—R_{A}¹⁴, S—R_{A}¹⁴, NR_{A}¹⁵R_{A}¹⁶, SO₂NR_{A}¹⁵R_{A}¹⁶ or CO—NR_{A}¹⁵R_{A}¹⁶ as in each case described above for R_{A}¹.

Preferred radicals for R_{A}¹³ and R_{A}¹³* are the radicals hydrogen, F, Cl, a branched or unbranched, optionally substituted C₁-C₆-alkyl radical, optionally substituted aryl or arylalkyl or a radical CO—O—R_{A}¹⁴, O—R_{A}¹⁴, NR_{A}¹⁵R_{A}¹⁶, SO₂—NR_{A}¹⁵R_{A}¹⁶ or CO—NR_{A}¹⁵R_{A}¹⁶.

A branched or unbranched, optionally substituted C₁-C₆-alkyl, C₃-C₇-cycloalkyl, alkylenecycloalkyl, alkylene-C₁-C₄-alkoxy, C₂-C₆-alkenyl or C₂-C₆-alkynyl radical for R_{A}¹⁴ in structural element A is understood as meaning, for example, the corresponding radicals described above for R_{G}⁷.

Optionally substituted aryl, arylalkyl, hetaryl or alkylhetaryl radicals for R_{A}¹⁴ in structural element A are understood as meaning, for example, the corresponding radicals described above for R_{G}⁷.

Preferred radicals for R_{A}¹⁴ are hydrogen, a branched or unbranched, optionally substituted C₁-C₆-alkyl radical and optionally substituted benzyl.

A branched or unbranched, optionally substituted C₁-C₆-alkyl or arylalkyl radical or an optionally substituted C₃-C₇-cycloalkyl, aryl, hetaryl or hetarylalkyl radical for R_{A}¹⁵ or R_{A}¹⁶ independently of one another is understood as meaning, for example, the corresponding radicals described above for R_{A}¹⁴.

The branched or unbranched, optionally substituted CO—C₁-C₆-alkyl, SO₂—C₁-C₆-alkyl, COO—C₁-C₆-alkyl, CO—NH—C₁-C₆-alkyl, COO-alkylenearyl, CO—NH-alkylenearyl, CO—NH-alkylenehetaryl or SO₂-alkylenearyl radicals or the optionally substituted CO-aryl, SO₂-aryl, CO—NH-aryl, CO—NH-hetaryl or CO-hetaryl radicals for R_{A}¹⁵ or R_{A}¹⁶ are composed, for example, of the corresponding groups —CO—, —SO₂—, —CO—O—, —CO—NH— and the corresponding branched or unbranched, optionally substituted C₁-C₆-alkyl, hetarylalkyl or arylalkyl radicals or the corresponding optionally substituted aryl or hetaryl radicals described above.

A radical —(CH₂)ₙ—(X_{A})ⱼ—R_{A}¹² for R_{A}³ or R_{A}⁴ independently of one another is understood as meaning a radical which is composed of the corresponding radicals —(CH₂)ₙ—, (X_{A})ⱼ and R_{A}¹². Here, n can be: 0, 1, 2 or 3 and j can be: 0 or 1.

X_{A} is a doubly bonded radical selected from the group —CO—, —CO—N(R_{X}¹)—, —N(R_{X}¹)—CO—, —N(R_{X}¹)—CO—N(R_{X}¹*)—, —N(R_{X}¹)—CO—O—, —O—, —S—, —SO₂—, —SO₂—N(R_{X}¹)—, —SO₂—O—, —CO—O—, —O—CO—, —O—CO—N(R_{X}¹)—, —N(R_{X}¹)— and —N(R_{X}¹)—SO₂—.

R_{A}¹² is hydrogen,

a branched or unbranched, optionally substituted C₁-C₆-alkyl radical, as described above for R_{G}⁷.
a C₂-C₆-alkynyl or C₂-C₆-alkenyl radical optionally substituted by C₁-C₄-alkyl or aryl,
or a 3- to 6-membered, saturated or unsaturated heterocycle which is substituted by up to three identical or different radicals and can contain up to three different or identical heteroatoms O, N, S, such as optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 2-(1,3,4-thiadiazolyl), 2-(1,3,4)-oxadiazolyl, 3-isoxazolyl, 4-isoxazolyl, 5-isoxazolyl, triazinyl.

Further, R_{A}¹² and R_{X}¹ or R_{X}¹* can together form a saturated or unsaturated C₃-C₇-heterocycle which can optionally contain up to two further heteroatoms selected from the group consisting of O, S and N.

Preferably, the radical R_{A}¹² together with the radical R_{X}¹ or R_{X}¹* forms a cyclic amine as the C₃-C₇-heterocycle in the case where the radicals are bonded to the same nitrogen atom, such as N-pyrrolidinyl, N-piperidinyl, N-hexahydroazepinyl, N-morpholinyl or N-piperazinyl, where in heterocycles which carry free amine protons, such as N-piperazinyl, the free amine protons can be replaced by customary amine protective groups, such as methyl, benzyl, Boc (tert-butoxycarbonyl), Z (benzyloxycarbonyl), tosyl, —SO₂—C₁-C₄-alkyl, —SO₂-phenyl or —SO₂-benzyl.

A branched or unbranched, optionally substituted C₁-C₆-alkyl, C₂-C₁₂-alkynyl, preferably C₂-C₆-alkynyl or C₂-C₆-alkenyl radical, an optionally substituted C₃-C₇-cycloalkyl, aryl, arylalkyl or hetaryl radical for R_{X}¹ and R_{X}¹* independently of one another is understood as meaning, for example, the corresponding radicals described above for R_{G}⁷.

Preferred, branched or unbranched, optionally substituted C₁-C₆-alkoxyalkyl for R_{X}¹ and R_{X}¹* are independently of one another methoxymethylene, ethoxymethylene, t-butoxymethylene, methoxyethylene or ethoxyethylene.

Preferred, branched or unbranched, optionally substituted radicals CO—C₁-C₆-alkyl, CO—O—C₁-C₆-alkyl, SO₂—C₁-C₆-alkyl, CO—O-alkylenearyl, CO-alkylenearyl, CO-aryl, SO₂-aryl, CO-hetaryl or SO₂-alkylenearyl are preferably composed of the C₁-C₆-alkyl, arylalkyl, aryl or hetaryl radicals and the radicals —CO—, —O—, —SO₂— described above.

Preferred radicals for R_{X}¹ and R_{X}¹* are independently of one another hydrogen, methyl, cyclopropyl, allyl and propargyl.

R_{A}³ and R_{A}⁴ can further together form a 3- to 8-membered saturated, unsaturated or aromatic N heterocycle which can additionally contain two further, identical or different heteroatoms O, N or S, where the cycle can be optionally substituted or a further, optionally substituted, saturated, unsaturated or aromatic cycle can be fused to this cycle,

R_{A}⁵ is a branched or unbranched, optionally substituted C₁-C₆-alkyl, arylalkyl, C₁-C₄-alkyl-C₃-C₇-cycloalkyl or C₃-C₇-cycloalkyl radical or an optionally substituted aryl, hetaryl, heterocycloalkyl or heterocycloalkenyl radical, such as described above for R_{G}⁷.

R_{A}⁶ and R_{A}⁶* are independently of one another hydrogen, a branched or unbranched, optionally substituted

C₁-C₄-alkyl radical, such as optionally substituted methyl, ethyl, propyl, 1-methylethyl, butyl, 1-methylpropyl, 2-methylpropyl or 1,1-dimethylethyl,
—CO—O—C₁-C₄-alkyl or —CO—C₁-C₄-alkyl radical such as composed of the group —CO—O— or —CO— and the C₁-C₄-alkyl radicals described above,
arylalkyl radical, as described above for R_{G}⁷,
—CO—O-alkylenearyl or —CO-alkylenearyl radical such as composed of the group —CO—O— or —CO— and the arylalkyl radicals described above,
—CO—O-allyl or —CO-allyl radical,
or C₃-C₇-cycloalkyl radical, such as described above for R_{G}⁷.

Further, both radicals R_{A}⁶ and R_{A}⁶* in structural element I_{A}⁷ can together form an optionally substituted, saturated, unsaturated or aromatic heterocycle which, in addition to the ring nitrogen, can contain up to two further different or identical heteroatoms O, N, S.

R_{A}⁷ is hydrogen, —OH, —CN, —CONH₂, a branched or unbranched, optionally substituted C₁-C₄-alkyl radical, for example as described above for R_{A}⁶, C₁-C₄-alkoxy, arylalkyl or C₃-C₇-cycloalkyl radical, for example as described above for R_{L}¹⁴, a branched or unbranched, optionally substituted —O—CO—C₁-C₄-alkyl radical, which is composed of the group —O—CO— and, for example, of the C₁-C₄-alkyl radicals mentioned above or an optionally substituted —O-alkylenearyl, —O—CO-aryl, —O—CO-alkylenearyl or —O—CO-allyl radical which is composed of the groups —O— or —O—CO— and, for example, of the corresponding radicals described above for R_{G}⁷.

Further, both radicals R_{A}⁶ and R_{A}⁷ can together form an optionally substituted unsaturated or aromatic heterocycle which, in addition to the ring nitrogen, can contain up to two further different or identical heteroatoms O, N, S.

For RAS in structural element A, the branched or unbranched, optionally substituted C₁-C₄-alkyl radical or an optionally substituted aryl or arylalkyl radical is understood as meaning, for example, the corresponding radicals described above for R_{A}¹⁵, where the radicals CO—C₁-C₄-alkyl, SO₂—C₁-C₄-alkyl, CO—O—C₁-C₄-alkyl, CO-aryl, SO₂-aryl, CO—O-aryl, CO-alkylenearyl, SO₂-alkylenearyl or CO—O-alkylenearyl are composed analogously to the other composed radicals of the group consisting of CO, SO₂ and COO and, for example, of the corresponding C₁-C₄-alkyl, aryl or arylalkyl radicals described above for R_{A}¹⁵ and these radicals can be optionally substituted.

In each case, for R_{A}⁹ or R_{A}¹⁰ independently of one another, a branched or unbranched, optionally substituted C₁-C₆-alkyl radical or an optionally substituted aryl, arylalkyl, hetaryl or C₃-C₇-cycloalkyl radical is understood as meaning, for example, the corresponding radicals described above for R_{A}¹⁴, preferably methyl or trifluoromethyl.

In each case, for R_{A}⁹ or R_{A}¹⁰ independently of one another, a radical CO—O—R_{A}¹⁴, O—R_{A}¹⁴, S—R_{A}¹⁴, SO₂—NR_{A}¹⁵R_{A}¹⁶, NR_{A}¹⁵R_{A}¹⁶ or CO—NR_{A}¹⁵R_{A}¹⁶ is understood as meaning, for example, the corresponding radicals described above for R_{A}¹³.

Further, both radicals R_{A}⁹ and R_{A}¹⁰ together in structural element I_{A}¹⁴ can form a 5- to 7-membered saturated, unsaturated or aromatic carbocycle or heterocycle, which can contain up to three different or identical heteroatoms O, N, S and is optionally substituted by up to three identical or different radicals.

Substituents in this case are in particular understood as meaning halogen, CN, a branched or unbranched, optionally substituted C₁-C₄-alkyl radical, such as methyl or trifluoromethyl or the radicals O—R_{A}¹⁴, S—R_{A}¹⁴, NR_{A}¹⁵R_{A}¹⁶, CO—NR_{A}¹⁵R_{A}¹⁶ or —((R_{A}⁸)HN)C═N—R_{A}⁷.

A branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶ for R_{A}¹¹ is understood, for example, as meaning the corresponding radicals described above for R_{A}⁹.

Further, in structural element I_{A}¹⁶, both radicals R_{A}⁹ and R_{A}¹⁷ together can form a 5- to 7-membered saturated, unsaturated or aromatic heterocycle which, in addition to the ring nitrogen, can contain up to three different or identical heteroatoms O, N, S and is optionally substituted by up to three identical or different radicals.

A branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹¹ for R_{A}¹⁸ and R_{A}¹⁹ independently of one another is understood as meaning, for example, the radicals described above for R_{G}¹², preferably hydrogen or a branched or unbranched, optionally substituted C₁-C₈-alkyl radical.

Z¹, Z², Z³, Z⁴ are independently of one another nitrogen, C—H, C-halogen, such as C—F, C—Cl, C—Br or C—I or a branched or unbranched, optionally substituted C—C₁-C₄-alkyl radical which is composed of a carbon radical and, for example, a C₁-C₄-alkyl radical described above for R_{A}⁶ or a branched or unbranched optionally substituted C—C₁-C₄-alkoxy radical which is composed of a carbon radical and, for example, a C₁-C₄-alkoxy radical described above for R_{A}⁷.

Z⁵ is oxygen, sulfur or a radical NR_{A}⁸.

Preferred structural elements A are composed of at least one preferred radical of the radicals belonging to the structural element A, while the remaining radicals are widely variable.

Particularly preferred structural elements A are composed of the preferred radicals of the structural element A.

In a preferred embodiment, the spacer structural element E is understood as meaning a structural element that consists of a branched or unbranched aliphatic C₂-C₃₀-hydrocarbon radical which is optionally substituted and contains heteroatoms and/or of a 4- to 20-membered aliphatic or aromatic mono- or polycyclic hydrocarbon radical which is optionally substituted and contains heteroatoms.

In a further preferred embodiment, the spacer structural element E is composed of two to four substructural elements, selected from the group consisting of E¹ and E², where the sequence of linkage of the substructural elements is arbitrary and E¹ and E² have the following meanings:
E¹ is a substructural element of the formula I_{E1}

—(Y_{E})ₖ₁—(CR_{E}¹R_{E}²)_{c}-(Q_{E})ₖ₂-(CR_{E}³R_{E}⁴)_{d}— I_{E1}
and
E² is a substructural element of the formula I_{E2}

—(NR_{E}¹¹)ₖ₃—(CR_{E}⁵R_{E}⁶)_{f}-(Z_{E})ₖ₄-(CR_{E}⁷R_{E}⁸)_{g}—(X_{E})ₖ₅—(CR_{E}⁹R_{E}¹⁰)ₕ—(NR_{E}¹¹*)ₖ₆— I_{E2},
where
c, d, f, g, h
   independently of one another are 0, 1 or 2,
k1, k2, k3, k4, k5, k6
   independently of one another are 0 or 1,
X_{E}, Q_{E}
   independently of one another are an optionally substituted 4- to 11-membered mono- or polycyclic, aliphatic or aromatic hydrocarbon which can contain up to 6 double bonds and up to 6 identical or different heteroatoms selected from the group N, O and S, where the ring carbons and/or the ring nitrogens can optionally be substituted,
Y_{E}, Z_{E}
   independently of one another are CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴, C(═CR_{E}¹³R_{E}¹⁴), CR_{E}¹³═CR_{E}¹⁴, —CR_{E}¹³(OR_{E}¹⁵)—CHR_{E}¹⁴— or —CHR_{E}¹³—CR_{E}¹⁴(OR_{E}¹⁵)—,
R_{E}¹, R_{E}², R_{E}³, R_{E}⁴, R_{E}⁵, R_{E}⁶, R_{E}⁷, R_{E}⁸, R_{E}⁹, R_{E}¹⁰
   independently, of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)ₓ—(W_{E})_{z}—R_{E}¹⁷, an optionally substituted C₃-C₇-cycloalkyl, aryl, arylalkyl, hetaryl or hetarylalkyl radical, or
   independently of one another in each case two radicals R_{E}¹ and R_{E}² or R_{E}³ and R_{E}⁴ or R_{E}⁵ and R_{E}⁶ or R_{E}⁷ and R_{E}⁸ or R_{E}⁹ and R_{E}¹⁰ together are a 3- to 7-membered, optionally substituted, saturated or unsaturated carbocycle or heterocycle which can contain up to three heteroatoms selected from the group O, N and S,
x is 0, 1, 2, 3 or 4,
z is 0 or 1,
W_{E} is —CO—, —CO—N(R_{W}²)—, —N(R_{W}²)—CO—, —N(R_{W}²)—CO—N(R_{W}²*)—, —N(R_{W}²)—CO—O—, —O—, —S—, —SO₂—, —SO₂—N(R_{W}²)—, —SO₂—O—, —CO—O—, —O—CO—, —O—CO—N(R_{W}²)—, —N(R_{W}²)— or —N(R_{W}²)—SO₂—,
R_{W}², R_{W}²*
   independently of one another are hydrogen, a branched or unbranched, optionally substituted C₁-C₆-alkyl, C₂-C₆-alkenyl, C₂-C₈-alkynyl, CO—C₁-C₆-alkyl CO—O—C₁-C₆-alkyl or SO₂—C₁-C₆-alkyl radical or an optionally substituted hetaryl, hetarylalkyl, arylalkyl, C₃-C₇-cycloalkyl, CO—O-alkylenearyl, CO-alkylenearyl, CO-aryl, SO₂-aryl, CO-hetaryl or SO₂-alkylenearyl radical,
R_{E}¹⁷ is hydrogen, a hydroxyl group, CN, halogen, a branched or unbranched, optionally substituted C₁-C₆-alkyl radical, an optionally substituted C₃-C₇-cycloalkyl, aryl, hetaryl or arylalkyl radical, a C₂-C₆-alkynyl or C₂-C₆-alkenyl radical optionally substituted by C₁-C₄-alkyl or aryl, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocycle substituted by up to three identical or different radicals, which can contain up to three different or identical heteroatoms O, N, S, where two radicals together can be a fused, saturated, unsaturated or aromatic carbocycle or heterocycle which can contain up to three different or identical heteroatoms O, N, S and the cycle can optionally be substituted or a further, optionally substituted, saturated, unsaturated or aromatic cycle can be fused to this cycle, or the radical R_{E}¹⁷ forms, together with R_{W}² or R_{W}²*, a saturated or unsaturated C₃-C₇-heterocycle which can optionally contain up to two further heteroatoms selected from the group O, S and N,
R_{E}¹¹, R_{E}¹¹*
   independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl, CO—NH—C₁-C₆-alkoxyalkyl, CO—NH—C₁-C₆-alkyl or SO₂—C₁-C₆-alkyl radical or an optionally substituted hetaryl, arylalkyl, C₃-C₇-cycloalkyl, CO—O-alkylenearyl, CO—NH-alkylenearyl, CO-alkylenearyl, CO-aryl, CO—NH-aryl, SO₂-aryl, CO-hetaryl, SO₂-alkylenearyl, SO₂-hetaryl or SO₂-alkylenehetaryl radical,
R_{E}¹² is hydrogen, a branched or unbranched, optionally substituted C₁-C₆-alkyl, C₂-C₆-alkenyl, C₂-C₆-alkynyl radical, an optionally substituted C₃-C₇-cycloalkyl, hetaryl, arylalkyl or hetarylalkyl radical or a radical CO—R_{E}¹⁶, COOR_{E}¹⁶ or SO₂—R_{E}¹⁶,
R_{E}¹³, R_{E}¹⁴
   independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁵ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁶ is hydrogen, a hydroxyl group, a branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical.

The coefficient c is preferably 0 or 1, the coefficient d is preferably 1 or 2, and the coefficients f, g, h independently of one another are preferably 0 or 1.

An optionally substituted 4- to 11-membered mono- or polycyclic aliphatic or aromatic hydrocarbon which can contain up to 6 double bonds and up to 6 identical or different heteroatoms selected from the group N, O, S, where the ring carbons or ring nitrogens can optionally be substituted, for Q_{E} and X_{E} independently of one another is preferably understood as meaning optionally substituted arylene, such as optionally substituted phenylene or naphthylene, or optionally substituted hetarylene such as the radicals

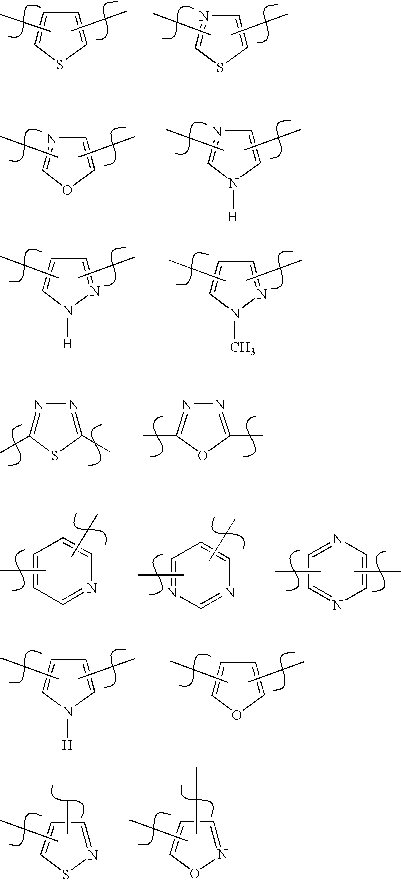

and their substituted or fused derivatives, or radicals of the formulae I_{E}¹ to I_{E}¹¹,

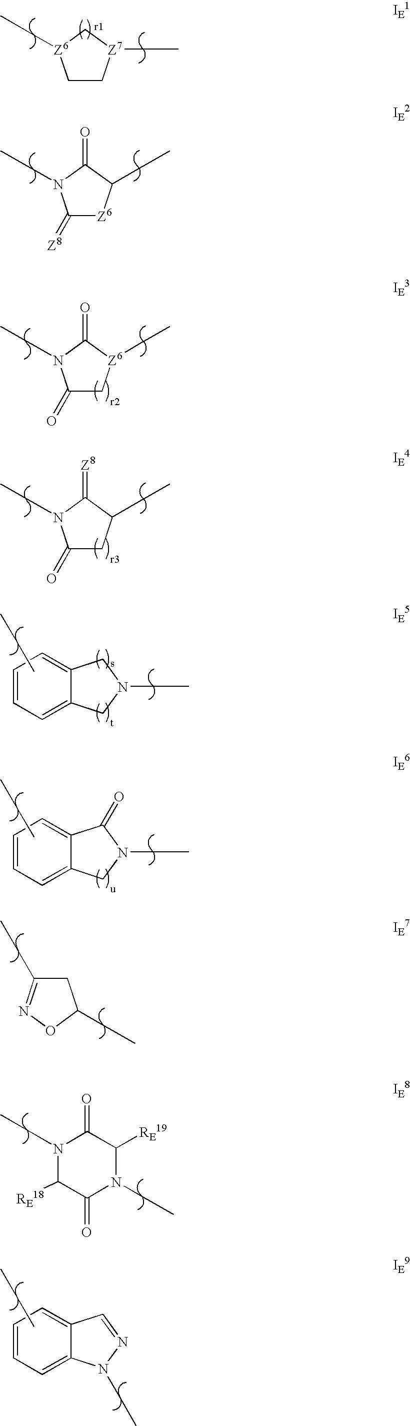

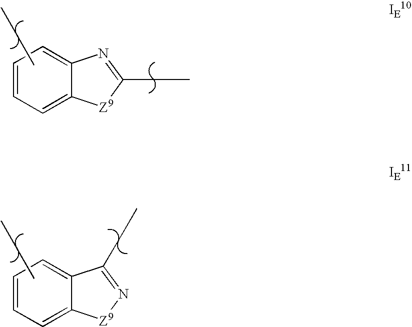

where the incorporation of the radicals can take place in both orientations. Aliphatic hydrocarbons are understood as meaning, for example, saturated and unsaturated hydrocarbons.

Z⁶ and Z⁷ are independently of one another CH or nitrogen.

Z⁸ is oxygen, sulfur or NH.

Z⁹ is oxygen, sulfur or NR_{E}²⁰.

r1, r2, r3 and t are independently of one another 0, 1, 2 or 3.

s and u are independently of one another 0, 1 or 2.

Particularly preferably, X_{E} and Q_{E} independently of one another are optionally substituted phenylene, a radical

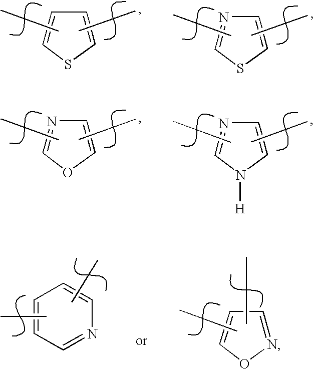

and their substituted or fused derivatives, or radicals of the formulae I_{E1}, I_{E}², I_{E}³, I_{E}⁴ and I_{E}⁷, where the incorporation of the radicals can take place in both orientations.

R_{E}¹⁸ and R_{E}¹⁹ are independently of one another hydrogen, —NO₂, —NH₂, —CN, —COOH, a hydroxyl group, halogen, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical, as in each case described above.

R_{E}²⁰ is independently of one another hydrogen, a branched or unbranched, optionally substituted C₁-C₆-alkyl, C₁-C₆-alkoxyalkyl, C₃-C₁₂-alkynyl, CO—C₁-C₆-alkyl, CO—O—C₁-C₆-alkyl or SO₂—C₁-C₆-alkyl radical or an optionally substituted C₃-C₇-cycloalkyl, aryl, arylalkyl, CO—O-alkylenearyl, CO-alkylenearyl, CO-aryl, SO₂-aryl, hetaryl, CO-hetaryl or SO₂-alkylenearyl radical, preferably hydrogen or a branched or unbranched, optionally substituted C₁-C₆-alkyl radical.

Y_{E} and Z_{E} are independently of one another CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴, C(═CR_{E}¹³R_{E}¹⁴), CR_{E}¹³═CR_{E}¹⁴, —CR_{E}¹³(OR_{E}¹⁵)—CHR_{E}¹⁴— or —CHR_{E}¹³—CR_{E}¹⁴(OR_{E}¹⁵)—, preferably CO, SO₂ and oxygen.

R_{E}¹² is hydrogen, a branched or unbranched, optionally substituted C₁-C₆-alkyl, C₂-C₆-alkenyl or C₂-C₈-alkynyl radical or an optionally substituted C₃-C₇-cycloalkyl, hetaryl, arylalkyl or hetarylalkyl radical, such as correspondingly described above for R_{G}⁷ or a radical CO—R_{E}¹⁶, COOR_{E}¹⁶ or SO₂—R_{E}¹⁶, preferably hydrogen, methyl, allyl, propargyl and cyclopropyl.

A branched or unbranched, optionally substituted C₁-C₆-alkyl, C₂-C₆-alkenyl or C₂-C₆-alkynyl radical or an optionally substituted C₃-C₇-cycloalkyl, aryl, arylalkyl, hetaryl or hetarylalkyl radical for R_{E}¹³, R_{E}¹⁴ or R_{E}¹⁵ independently of one another is understood as meaning, for example, the corresponding radicals described above for R_{G}⁷.

A branched or unbranched, optionally substituted C₁-C₄-alkoxy radical for R_{E}¹³ or R_{E}¹⁴ independently of one another is understood as meaning, for example, the C₁-C₄-alkoxy radicals described above for R_{A}¹⁴.

Preferred alkylenecycloalkyl radicals for R_{E}¹³, R_{E}¹⁴ or R_{E}¹⁵ independently of one another are, for example, the C₁-C₄-alkylene-C₃-C₇-cycloalkyl radicals described above for R_{G}⁷.

A branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical for R_{E}¹⁶ is understood as meaning, for example, the corresponding radicals described above for R_{G}¹¹.

A branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical for R_{E}¹, R_{E}², R_{E}³, R_{E}⁴, R_{E}⁵, R_{E}⁶, R_{E}⁷, R_{E}⁸, R_{E}⁹ or R_{E}¹⁰ independently of one another is understood as meaning, for example, the corresponding radicals mentioned above for R_{G}¹.

Further, two radicals R_{E}³ and R_{E}⁴ or R_{E}⁵ and R_{E}⁶ or R_{E}⁷ and R_{E}⁸ or R_{E}⁹ and R_{E}¹⁰ can in each case independently of one another together form a 3- to 7-membered, optionally substituted, saturated or unsaturated carbo- or heterocycle which can contain up to three heteroatoms from the group O, N and S.

The radical —(CH₂)ₓ—(W_{E})_{z}—R_{E}¹⁷ is composed of a C₀-C₄-alkylene radical, optionally a bonding element W_{E} selected from the group —CO—, —CO—N(R_{W}²)—, —N(R_{W}²)—CO—, —N(R_{W}²)—CO—N(R_{W}²*)—, —N(R_{W}²)—CO—O—, —O—, —S—, —SO₂—, —SO₂—N(R_{W}²)—, —SO₂—O—, —CO—O—, —O—CO—, —O—CO—N(R_{W}²)—, —N(R_{W}²)— or —N(R_{W}²)—SO₂—, preferably selected from the group —CO—N(R_{W}²)—, —N(R_{W}²)—CO—, —O—, —SO₂—N(R_{W}²)—, —N(R_{W}²)— or —N(R_{W}²)—SO₂—, and the radical R_{E}¹⁷, where

### R_{W}² and R_{W}²*

independently of one another are hydrogen, a branched or unbranched, optionally substituted C₁-C₆-alkyl, C₂-C₆-alkenyl, C₂-C₈-alkynyl, CO—C₁-C₆-alkyl, CO—O—C₁-C₆-alkyl or SO₂—C₁-C₆-alkyl radical or an optionally substituted hetaryl, hetarylalkyl, arylalkyl, C₃-C₇-cycloalkyl, CO—O-alkylenearyl, CO-alkylenearyl, CO-aryl, SO₂-aryl, CO-hetaryl or SO₂-alkylenearyl radical, preferably independently of one another are hydrogen, methyl, cyclopropyl, allyl, propargyl, and

### R_{E}¹⁷

is hydrogen, a hydroxyl group, CN, halogen, a branched or unbranched, optionally substituted C₁-C₆-alkyl radical, an optionally substituted C₃-C₇-cycloalkyl, aryl, hetaryl or arylalkyl radical, a C₂-C₆-alkynyl or C₂-C₆-alkenyl radical optionally substituted by C₁-C₄-alkyl or aryl, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3 to 8-membered, saturated or unsaturated heterocycle which is substituted by up to three identical or different radicals and can contain up to three different or identical heteroatoms O, N, S, where two radicals together can be a fused, saturated, unsaturated or aromatic carbocycle or heterocycle which can contain up to three different or identical heteroatoms O, N, S, and the cycle can be optionally substituted or a further, optionally substituted, saturated, unsaturated or aromatic cycle can be fused to this cycle, such as optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 2-(1,3,4-thiadiazolyl), 2-(1,3,4)-oxadiazolyl, 3-isoxazolyl, 4-isoxazolyl, 5-isoxazolyl or triazinyl.

Further, R_{E}¹⁷ and R_{W}² or R_{W}²* can together form a saturated or unsaturated C₃-C₇-heterocycle which can optionally contain up to two further heteroatoms selected from the group consisting of O, S and N.

Preferably, the radicals R_{E}¹⁷ and R_{W}² or R_{W}²* together form a cyclic amine as the C₃-C₇-heterocycle in the case where the radicals are bonded to the same nitrogen atom, such as N-pyrrolidinyl, N-piperidinyl, N-hexahydroazepinyl, N-morpholinyl or N-piperazinyl where in heterocycles which carry free amine protons, such as N-piperazinyl, the free amine protons can be replaced by customary amine protective groups, such as methyl, benzyl, Boc (tert-butoxycarbonyl), Z (benzyloxycarbonyl), tosyl, —SO₂—C₁-C₄-alkyl, —SO₂-phenyl or —SO₂-benzyl.

Preferred radicals for R_{E}¹, R_{E}², R_{E}³, R_{E}⁴, R_{E}⁵, R_{E}⁶, R_{E}⁷, R_{E}⁸, R_{E}⁹ or R_{E}¹⁰ are independently of one another hydrogen, halogen, a branched or unbranched, optionally substituted C₁-C₆-alkyl radical, optionally substituted aryl or the radical —(CH₂)ₓ—(W_{E})_{z}—R_{E}¹⁷.

Particularly preferred radicals for R_{E}¹, R_{E}², R_{E}³, R_{E}⁴, R_{E}⁵, R_{E}⁶, R_{E}⁷, R_{E}⁸, R_{E}⁹ or R_{E}¹⁰ are independently of one another hydrogen, F, a branched or unbranched, optionally substituted C₁-C₄-alkyl radical, in particular methyl.

A branched or unbranched, optionally substituted C₁-C₆-alkyl, C₁-C₆-alkoxyalkyl, C₂-C₆-alkenyl, C₂-C₁₂-alkynyl or arylalkyl radical or an optionally substituted aryl, hetaryl or C₃-C₇-cycloalkyl for R_{E}¹¹ and R_{E}¹¹* in structural element E independently of one another is understood as meaning, for example, the corresponding radicals described above for R_{G}⁷.

The branched or unbranched, optionally substituted radicals CO—C₁-C₆-alkyl, CO—O—C₁-C₆-alkyl, CO—NH—C₁-C₆-alkoxyalkyl, CO—NH—C₁-C₆-alkyl or SO₂—C₁-C₆-alkyl radical or the optionally substituted radicals CO—O-alkylenearyl, CO—NH-alkylenearyl, CO-alkylenearyl, CO-aryl, CO—NH-aryl, SO₂-aryl, CO-hetaryl, SO₂-alkylenearyl, SO₂-hetaryl or SO₂-alkylenehetaryl for R_{E}¹¹ and R_{E}¹¹* independently of one another are composed, for example, of the corresponding groups CO, COO, CONH or SO₂ and the corresponding radicals mentioned above.

Preferred radicals for R_{E}¹¹ or R_{E}¹¹* are independently of one another hydrogen, a branched or unbranched, optionally substituted C₁-C₆-alkyl, C₁-C₆-alkoxy, C₂-C₆-alkenyl, C₂-C₁₂-alkynyl or arylalkyl radical, or an optionally substituted hetaryl or C₃-C₇-cycloalkyl radical.

Particularly preferred radicals for R_{E}¹¹ or R_{E}¹¹* are hydrogen, methyl, cyclopropyl, allyl or propargyl.

In a particularly preferred embodiment of structural element E₁, structural element E₁ is a radical —CH₂—CH₂—CO—, —CH₂—CH₂—CH₂—CO— or a C₁-C₅-alkylene radical.

In a particularly preferred embodiment of structural element E, the spacer structural element E used is a structural element of the formula I_{E1E2}

-E₂-E₁- I_{E1E2}

where the structural elements E₂ and E₁ have the meanings described above.

Preferred structural elements E are composed of at least one preferred radical of the radicals belonging to structural element E, while the remaining radicals are widely variable.

Particularly preferred structural elements E are composed of the preferred radicals of structural element E.

Preferred structural elements B are composed either of the preferred structural element A, while E is widely variable, or of the preferred structural element E, while A is widely variable.

In a further preferred embodiment, the structural element E used is the structural element E′ described below for the novel compounds of the formula I′.

In a further preferred embodiment, the structural element G used is the structural element G′ described below for the novel compounds of the formula I′.

The compounds of the formula I, and also the intermediates for their preparation, can have one or more asymmetric substituted carbon atoms. The compounds can be present as pure enantiomers or pure diastereomers or as a mixture thereof. The use of an enantiomerically pure compound as the active compound is preferred.

The compounds of the formula I can also be present in other tautomeric forms.

The compounds of the formula I can also be present in the form of physiologically tolerable salts.

The compounds of the formula I can also be present as prodrugs in a form in which the compounds of the formula I are liberated under physiological conditions. By way of example, reference may be made here to the group T in structural element L, which in some cases contains groups which are hydrolyzable to the free carboxylic acid group under physiological conditions. Derivatized structural elements B or A are also suitable which liberate the structural element B or A respectively under physiological conditions.

In preferred compounds of the formula I, in each case one of the three structural elements B, G or L has the preferred range, while the remaining structural elements are widely variable.

In particularly preferred compounds of the formula I, in each case two of the three structural elements B, G and L have the preferred range, while the remaining structural elements are widely variable.

In very particularly preferred compounds of the formula I, in each case all three structural elements B, G and L have the preferred range, while the remaining structural element is widely variable.

Preferred compounds of the formula I contain, for example, the preferred structural element G, while the structural elements B and L are widely variable.

In particularly preferred compounds of the formula I, for example, B is replaced by the structural element A-E- and the compounds contain, for example, the preferred structural element G and the preferred structural element A, while the structural elements E and L are widely variable.

Further particularly preferred compounds of the formula I contain, for example, the preferred structural element G and the preferred structural element A, while the structural elements E and L are widely variable.

The invention further relates to the use of the structural element of the formula I_{GL}

-G-L I_{GL}

for the preparation of compounds which bind to integrin receptors.

The compounds of the formula I bind to integrin receptors. The compounds of the formula I are therefore preferably suitable as integrin receptor ligands and for the production of drugs for treating diseases in which an integrin receptor is involved, in particular for treating diseases in which the interaction between integrins and their natural ligands is dysregulated, i.e. excessive or decreased.

Integrin receptor ligands are understood as meaning agonists and antagonists.

An excessive or decreased interaction is understood as meaning both an excessive or decreased expression of the natural ligand and/or of the integrin receptor and thus an excessive or decreased amount of natural ligand and/or integrin receptor or an increased or decreased affinity of the natural ligand for the integrin receptor.

The interaction between integrins and their natural ligands is dysregulated compared with the normal state, i.e. excessive or decreased, if this dysregulation does not correspond to the physiological state. An increased or decreased interaction can lead to pathophysiological situations.

The level of dysregulation which leads to a pathophysiological situation is dependent on the individual organism and on the site and nature of the disorder.

Preferred integrin receptors for which the compounds of the formula I according to the invention can be used are the α₅β₁, α₄β₁, α_{V}β₅ and α_{V}β₃ integrin receptors.

The compounds of the formula I particularly preferably bind to the α_{V}β₃ integrin receptor and can thus be particularly preferably used as ligands of the α_{V}β₃ integrin receptor and for the treatment of illnesses in which the interaction between α_{V}β₃ integrin receptor and its natural ligand is excessive or reduced.

The compounds of the formula I are preferably used for the treatment of the following illnesses:

cardiovascular disorders such as atherosclerosis, restenosis after vascular injury or stent implantation, and angioplasty (neointima formation, smooth muscle cell migration and proliferation),
acute kidney failure,
angiogenesis-associated microangiopathies such as diabetic angiopathies or retinopathy or rheumatoid arthritis,
blood platelet-mediated vascular occlusion, arterial thrombosis,
stroke, reperfusion damage after myocardial infarct or stroke, carcinomatous disorders, such as in tumor metastasis or in tumor growth (tumor-induced angiogenesis),
osteoporosis (bone resorption after chemotaxis and adhesion of osteoclasts to the bone matrix),
high blood pressure, psoriasis, hyperparathyroidism, Paget's disease, malignant hypercalcemia, metastatic osteolytic lesions, inflammation, wound healing, cardiac insufficiency, congestive heart failure CHF, as well as in
antiviral, antimycotic, antiparasitic or antibacterial therapy and prophylaxis (adhesion and internalization).

Furthermore, the invention relates in particular to the use of the compounds of the formula I as ligands of the α_{V}β₃ integrin receptor.

The invention furthermore relates to the novel compounds of the formula I′

A-E′-G′-L I′

where the structural elements A and L have the meanings described above. Preferred structural elements A and L are described.

Structural element E′ is composed of two to four substructural elements selected from the group consisting of E¹ and E², the linkage sequence of the substructural elements being arbitrary and E¹ and E² having the following meanings:
E¹ is a substructural element of the formula I_{E1}

—(Y_{E})ₖ₁—(CR_{E}¹R_{E}²)_{c}-(Q_{E})ₖ₂-(CR_{E}³R_{E}⁴)_{d}— I_{E1}
and
E² is a substructural element of the formula I_{E2}

—(NR_{E}¹¹)ₖ₃—(CR_{E}⁵R_{E}⁶)_{f}-(Z_{E})ₖ₄-(CR_{E}⁷R_{E}⁸)_{g}—(X)ₖ₅—(CR_{E}⁹R_{E}¹⁰)ₕ—(NR_{E}¹¹*)ₖ₆— I_{E2},

where all radicals and coefficients of structural element E′ have the meanings of structural element E described above, with the proviso that in the case
in which Y_{E} or Z_{E}=CO and a radical X_{E} or Q_{E} or an aromatic or heteroaromatic radical of the structural element A is bonded directly to Y_{E} or Z_{E}, a direct atomic bond from Y_{E} or Z_{E} to the structural element G′ is excluded.

In a further preferred embodiment of structural element E′, the structural element E′ used is a structural element of the formula I_{E1E2}

-E₂-E₁- I_{E1E2}
where E¹ and E² have the following meanings:
E¹ is a substructural element of the formula I_{E1}

—(Y_{E})ₖ₁—(CR_{E}¹R_{E}²)_{c}-(Q_{E})ₖ₂-(CR_{E}³R_{E}⁴)_{d}— I_{E1}
and
E² is a substructural element of the formula I_{E2}

—(NR_{E}¹¹)ₖ₃—(CR_{E}⁵R_{E}⁶)_{f}-(Z_{E})ₖ₄-(CR_{E}⁷R_{E}⁸)—(X_{E})ₖ₅—(CR_{E}⁹R_{E}¹⁰)ₕ—(NR_{E}¹¹*)ₖ₆— I_{E2},

where all radicals and coefficients of structural element E′ have the meanings of structural element E described above, with the proviso that in the case where
Y_{E}═CO,
k1 and k5=1 and
h and k6=0
the sum of the indices c, k2 and d must be other than 0
   and in the case where an aromatic or heteroaromatic radical from the structural element A is bonded directly to Y_{E} or Z_{E}, a direct atomic bond from Y_{E} or Z_{E} to the structural element G′ is excluded.

Further preferred embodiments of structural element E′ correspond to the preferred embodiments of structural element E with the proviso described above.

Structural element G′ is identical to structural element G, as described above, except for the radicals R_{G}¹² and R_{G}¹³. In structural element G′, the radicals R_{G}¹² and R_{G}¹³ of structural element G are replaced by the radicals R_{G′}¹² and R_{G′}¹³.

The radicals R_{G}¹² and R_{G′}¹³ in structural element G′ have the following meanings:

independently of one another hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹⁴,
where R_{G}¹⁴ is hydrogen, a branched or unbranched, optionally substituted C₁-C₈-alkyl, C₂-C₆-alkenyl, C₂-C₆-alkynyl or C₁-C₅-alkylene-C₁-C₄-alkoxy radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical.

Preferred radicals of R_{G′}¹² and R_{G}¹³ are the corresponding radicals described above for R_{G}¹² and R_{G}¹³.

Further preferred embodiments of the structural element G′ correspond to the preferred embodiments of structural element G.

The compounds of the formula I′, and also the intermediates for their preparation, can have one or more asymmetric substituted carbon atoms. The compounds can be present as pure enantiomers or pure diastereomers or as a mixture thereof. The use of an enantiomerically pure compound as active compound is preferred.

The compounds of the formula I′ can also be present in other tautomeric forms.

The compounds of the formula I′ can also be present in the form of physiologically tolerable salts.

The compounds of the formula I′ can also be present as prodrugs in a form in which the compounds of the formula I′ are liberated under physiological conditions. By way of example, reference may be made here to the group T in structural element L, which in some cases contains groups which are hydrolyzable to the free carboxylic acid group under physiological conditions. Derivatized structural elements A which liberate the structural element A under physiological conditions are also suitable.

Analogously to the compounds of the formula I, the following applies for the compounds of the formula I′ as mentioned above:

In preferred compounds of the formula I′, one of the four structural elements A, E′, G′ or L in each case have the preferred range, while the remaining structural elements are widely variable.

In particularly preferred compounds of the formula I′, two of the four structural elements A, E′, G′ or L in each case have the preferred range, while the remaining structural elements are widely variable.

In further particularly preferred compounds of the formula I′, three of the four structural elements A, E′, G′ or L in each case have the preferred range, while the remaining structural element is widely variable.

In very particularly preferred compounds of the formula I′, all four structural elements A, E′, G′ or L have the preferred range.

Preferred compounds of the formula I, have, for example, the preferred structural element G′, while the structural elements A, E′ and L are widely variable.

Further particularly preferred compounds of the formula I′ have, for example, the preferred structural element G′ and the preferred structural element A, while the structural elements E′ and L are widely variable.

Very particularly preferred compounds of the formula I′ are listed below, the number before the text block being the number of an individualized compound of the formula I′, and in the text block A-E′-G′-L the abbreviations separated by a bonding dash in each case being an individual structural element A, E′, G′ or L and the meaning of the abbreviations of the structural elements being explained after the table.

No. A-E′-G′-L
1 bhs-edia3-phen-es
2 2py-inda2-phen-es
3 bhs-35thirna2-meph-es
4 bim-dibema2-dmeph-es
5 2py-bam2-4-clph-es
6 2py-dibema2-dmeph-es
7 bhs-a24thima2-dmeph-es
8 bhs-aaf-phen-es
9 bhs-a24thima2-4-clph-es
10 bim-me42thiaz2-phen-es
11 2py-edia2-phen-es
12 bim-a24thima2-hdb-es
13 2py-apma2-4-clph-es
14 gua-chex2-phen-es
15 bhs-dibema2-4-clph-es
16 bhs-bam2-phen-es
17 bim-a23thima2-4-clph-es
18 bim-dibema2-phen-es
19 bim-bam2-4-clph-es
20 2py-pipa2-4-clph-es
21 2py-me25thima2-phen-es
22 gua-a24thima2-ioph-es
23 imhs-apma2-phen-es
24 2py-apma2-ioph-es
25 gua-edia3-phen-es
26 bhs-a23thima2-4-clph-es
27 2py-a24thima2-2pyph-es
28 2py-bam2-thoph-es
29 gua-apma2-phen-es
30 2py-a24thima2-reph-es
31 gua-hexa-phen-es
32 dimethpym-apma2-phen-es
33 2py-dibema2-meph-es
34 bhs-apma2-phen-es
35 bim-edia3-phen-es
36 gua-apma2-meph-es
37 bim-apma2-reph-es
38 2py-a24thima2-dmeph-es
39 gua-aaf-phen-es
40 gua-apma2-yrph-es
41 gua-pipeme2-phen-es
42 2py-35thima2-phen-es
43 gua-a24thima2-reph-es
44 bim-bam2-phen-es
45 gua-a24thima2-phen-ms
46 gua-apma2-reph-es
47 mam2py-a24thima2-phen-es
48 2py-a24thima2-phen-gs
49 2py-apma2-phen-nes
50 2py-a24thima2-4-clph-es
51 bhs-penta-phen-es
52 gua-35thima2-dmeph-es
53 bim-dibema2-thoph-es
54 bim-a24thima2-reph-es
55 2py-a23thima2-4-clph-es
56 gua-a23thima2-phen-es
57 dhim-a24thima2-phen-es
58 gua-penta-phen-es
59 bhs-a24thima2-reph-es
60 2py-a23thima2-meph-es
61 gua-prodia2-phen-es
62 bhs-apma2-reph-es
63 2py-apma2-meph-es
64 gua-bam2-4-clph-es
65 2py-me35thima2-phen-es
66 gua-apma2-2pyph-es
67 2py-35thima2-thoph-es
68 clim-a24thima2-phen-es
69 2py-buta-phen-es
70 am2py-apma2-phen-es
71 gua-a24thima2-dmeph-es
72 bhs-apma2-hdb-es
73 bhs-dibema2-thoph-es
74 bim-dibema2-meph-es
75 bhs-bam2-meph-es
76 bhs-apma2-yrph-es
77 bhs-apma2-dmeph-es
78 gua-me25thima2-phen-es
79 2py-a24thima2-meph-es
80 gua-inda2-phen-es
81 bhs-mepipe2-phen-es
82 bhs-a24thima2-phen-as
83 bim-pipa2-thoph-es
84 bhs-bam2-4-clph-es
85 bhs-a24thima2-phen-es
86 bhs-35thima2-phen-es
87 bim-penta-phen-es
88 bhs-apma2-dbph-es
89 gua-42thiaz2-phen-es
90 bhs-a24thima2-24pym-es
91 2py-dibema2-phen-es
92 bim-a24thima2-dm-es
93 bhs-pipa2-4-clph-es
94 2py-apma2-phen-ps
95 2py-apma2-dmeph-es
96 2py-mepipe2-phen-es
97 bhs-35thima2-4-clph-es
98 gua-apma2-phen-mals
99 gua-35thima2-4-clph-es
100 mam2py-apma2-phen-es
101 gua-buta-phen-es
102 2py-a23thima2-thoph-es
103 2py-pyma2-phen-es
104 gua-apma2-phen-gs
105 bim-apma2-phen-as
106 bhs-apma2-4-clph-es
107 bhs-a24thima2-4-pyph-es
108 thpym-apma2-phen-es
109 gua-pipa2-dmeph-es
110 amim-a24thima2-phen-es
111 bim-aepi2-phen-es
112 bim-a23thima2-thoph-es
113 bhs-a23thima2-thoph-es
114 gua-pdagk-phen-es
115 2py-hexa-phen-es
116 bhs-a24thima2-meph-es
117 gua-bam2-meph-es
118 2py-35thima2-meph-es
119 2py-pipa2-meph-es
120 bhs-a23thima2-phen-es
121 bim-pipeme2-phen-es
122 bhs-buta-phen-es
123 bhs-pipa2-dmeph-es
124 bhs-pipa2-meph-es
125 2py-35thima2-dmeph-es
126 bim-bam2-thoph-es
127 gua-35thima2-thoph-es
128 bim-edia2-phen-es
129 bim-apma2-phen-nes
130 gua-bam2-thoph-es
131 gua-bam2-phen-es
132 pippy-a24thima2-phen-es
133 gua-35thima2-phen-es
134 bim-a23thima2-phen-es
135 gua-dibema2-dmeph-es
136 bhs-apma2-phen-gs
137 bhs-apma2-thoph-es
138 2py-apma2-phen-es
139 im-a24thima2-phen-es
140 gua-aepi2-phen-es
141 2py-mea2-phen-es
142 gua-a24thima2-phen-es
143 2py-a24thima2-thaph-es
144 gua-apma2-4-clph-es
145 bhs-apma2-phen-f2es
146 bhs-inda2-phen-es
147 bim-a24thima2-dbph-es
148 bim-apma2-phen-ms
149 gua-a23thima2-thoph-es
150 pippy-apma2-phen-es
151 bhs-apma2-meph-es
152 2py-apma2-phen-as
153 gua-mea2-phen-es
154 bhs-me35thima2-phen-es
155 bhs-pdagk-phen-es
156 bim-42thiaz2-phen-es
157 2py-pipeme2-phen-es
158 bim-me25thima2-phen-es
159 gua-dibema2-phen-es
160 2py-apma2-oxph-es
161 bhs-a24thima2-3clph-es
162 bim-pyma2-phen-es
163 bhs-edia2-phen-es
164 imhs-a24thima2-phen-es
165 gua-dibema2-thoph-es
166 bim-a24thima2-4-clph-es
167 bim-apma2-phen-ps
168 gua-apma2-dm-es
169 2py-a24thima2-phen-mals
170 2py-dibema2-4-clph-es
171 bhs-dibema2-meph-es
172 bim-aof-phen-es
173 bhs-pipeme2-phen-es
174 gua-35thima2-meph-es
175 bim-aaf-phen-es
176 bim-dibema2-4-clph-es
177 bim-a24thima2-2pyph-es
178 bim-a24thima2-yrph-es
179 bim-35thima2-4-clph-es
180 bhs-aepi2-phen-es
181 bim-pipa2-phen-es
182 gua-a23thima2-dmeph-es
183 2py-a24thima2-yrph-es
184 gua-a24thima2-dmeoph-es
185 bhs-42thiaz2-phen-es
186 bim-mepipe2-phen-es
187 2py-chex2-phen-es
188 bhs-prodia2-phen-es
189 gua-bam2-dmeph-es
190 bhs-me25thima2-phen-es
191 thpym-a2.4thima2-phen-es
192 bim-pipa2-dmeph-es
193 bhs-dibema2-phen-es
194 gua-a24thima2-thoph-es
195 2py-pipa2-thoph-es
196 clim-apma2-phen-es
197 bhs-chex2-phen-es
198 gua-a24thima2-phen-nes
199 gua-a24thima2-meph-es
200 bhs-a24thima2-dmeoph-es
201 2py-apma2-24pym-es
202 2py-a24thima2-phen-pms
203 gua-a24thima2-4-piph-es
204 bim-apma2-3clph-es
205 gua-apma2-hdb-es
206 2py-bam2-meph-es
207 bhs-a24thima2-phen-nes
208 gua-pyma2-phen-es
209 bim-a24thima2-thoph-es
210 gua-mepipe2-phen-es
211 bim-apma2-4-clph-es
212 2py-42thiaz2-phen-es
213 bim-apma2-24pym-es
214 bhs-a23thima2-dmeph-es
215 gua-apma2-dbph-es
216 gua-me35thima2-phen-es
217 bim-35thima2-phen-es
218 gua-apma2-thaph-es
219 bim-35thima2-thoph-es
220 gua-apma2-phen-f2es
221 2py-apma2-3clph-es
222 gua-apma2-thoph-es
223 bim-pipa2-meph-es
224 2py-aof-phen-es
225 bhs-35thima2-dmeph-es
226 bhs-hexa-phen-es
227 bim-pipa2-4-clph-es
228 bhs-apma2-phen-pms
229 bim-a23thima2-dmeph-es
230 2py-me42thiaz2-phen-es
231 bim-a24thima2-phen-gs
232 bim-apma2-dmeph-es
233 gua-a23thima2-4-clph-es
234 bim-a24thima2-phen-mals
235 2py-apma2-phen-ms
236 bhs-a24thima2-ioph-es
237 bim-a24thima2-phen-es
238 2py-pdagk-phen-es
239 gua-a24thima2-phen-ps
240 2py-pipa2-phen-es
241 2py-aepi2-phen-es
242 2py-a24thima2-thoph-es
243 bhs-bam2-dmeph-es
244 bim-hexa-phen-es
245 bim-a24thima2-meph-es
246 bhs-me42thiaz2-phen-es
247 am2py-a24thima2-phen-es
248 bim-apma2-meph-es
249 bim-me35thima2-phen-es
250 gua-pipa2-phen-es
251 bhs-a24thima2-oxph-es
252 2py-pipa2-dmeph-es
253 2py-apma2-4-pyph-es
254 bhs-35thima2-thoph-es
255 gua-me42thiaz2-phen-es
256 bim-a24thima2-thaph-es
257 gua-a24thima2-phen-as
258 2py-a24thima2-phen-f2es
259 2py-a24thima2-dbph-es
260 bim-35thima2-meph-es
261 bim-apma2-phen-es
262 bim-a24thima2-phen-pms
263 bim-chex2-phen-es
264 bim-a24thima2-dmeph-es
265 bim-mea2-phen-es
266 2py-apma2-thoph-es
267 dimethpym-a24thima2-phen-es
268 2py-dibema2-thoph-es
269 2py-apma2-dmeoph-es
270 gua-dibema2-4-clph-es
271 bhs-pipa2-phen-es
272 gua-edia2-phen-es
273 gua-apma2-dmeph-es
274 2py-edia3-phen-es
275 gua-a23thima2-meph-es
276 bim-pdagk-phen-es
277 gua-apma2-phen-pms
278 bhs-mea2-phen-es
279 bim-35thima2-dmeph-es
280 bhs-aof-phen-es
281 2py-prodia2-phen-es
282 bim-inda2-phen-es
283 bhs-bam2-thoph-es
284 bim-apma2-4-pyph-es
285 2py-aaf-phen-es
286 2py-bam2-phen-es
287 bhs-apma2-dm-es
288 2py-penta-phen-es
289 gua-aof-phen-es
290 im-apma2-phen-es
291 gua-pipa2-4-clph-es
292 bim-apma2-ioph-es
293 bim-bam2-meph-es
294 gua-pipa2-meph-es
295 bhs-apma2-thaph-es
296 bhs-apma2-2pyph-es
297 bim-apma2-dmeoph-es
298 amim-apma2-phen-es
299 dhim-apma2-phen-es
300 bhs-a24thima2-phen-ps
301 2py-a23thima2-dmeph-es
302 gua-pipa2-thoph-es
303 bim-a23thima2-meph-es
304 2py-bam2-dmeph-es
305 bhs-a24thima2-thoph-es
306 bhs-apma2-phen-mals
307 bhs-a23thima2-meph-es
308 bim-buta-phen-es
309 2py-apma2-reph-es
310 gua-dibema2-meph-es
311 2py-a24thima2-hdb-es
312 gua-a24thima2-4-clph-es
313 bhs-pipa2-thoph-es
314 gua-a24thima2-3clph-es
315 gua-a24thima2-oxph-es
316 bim-bam2-dmeph-es
317 bim-apma2-thoph-es
318 bim-apma2-oxph-es
319 2py-a24thima2-phen-es
320 bhs-a24thima2-phen-ms
321 bim-prodia2-phen-es
322 2py-a24thima2-dm-es
323 bhs-pyma2-phen-es
324 bim-a24thima2-phen-f2es
325 2py-a23thima2-phen-es
326 gua-a24thima2-24pym-es
327 2py-35thima2-4-clph-es
328 bhs-dibema2-dmeph-es

In the above list, the following abbreviations are used for the structural units A, E′, G′ and L.

| A= | Abbreviation |
|---|---|
| 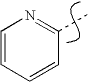 | 2py |
| 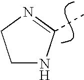 | dhim |
| 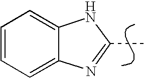 | bim |
| 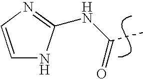 | imhs |
| 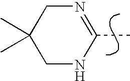 | dimethpym |
| 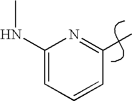 | mam2py |
| 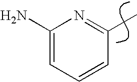 | am2py |
| 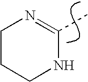 | thpym |
| 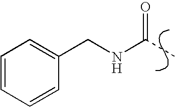 | bhs |
| 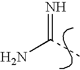 | gua |
| 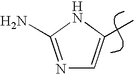 | amim |
| 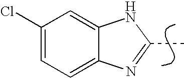 | clim |
| 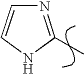 | im |
| 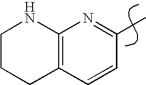 | pippy |

| E′= | Abbreviation |
|---|---|
| 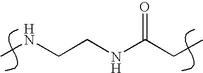 | edia2 |
| 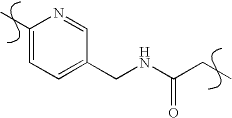 | pyma2 |
| 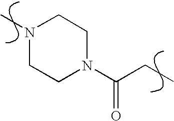 | pipa2 |
| 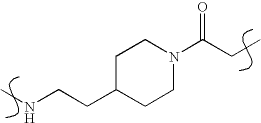 | aepi2 |
| 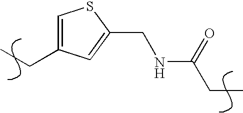 | me35thima2 |
| 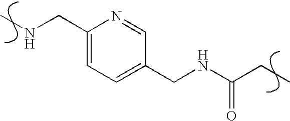 | dibema2 |
| 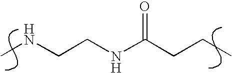 | edia3 |
| 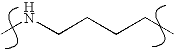 | buta |
| 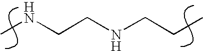 | aaf |
| 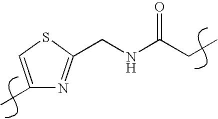 | 42thiaz2 |
| 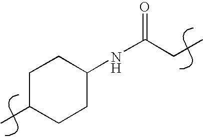 | chex2 |
| 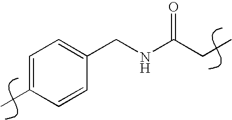 | bam2 |
| 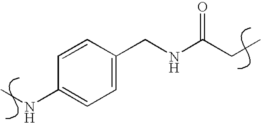 | apma2 |
| 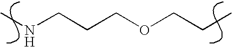 | pdagk |
| 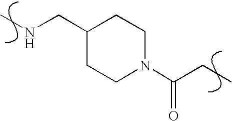 | mepipe2 |
| 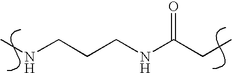 | prodia2 |
| 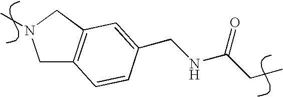 | inda2 |
| 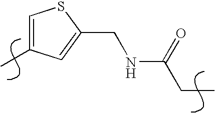 | 35thima2 |
| 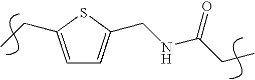 | me25thima2 |
| 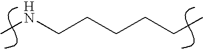 | penta |
| 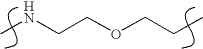 | aof |
| 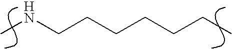 | hexa |
| 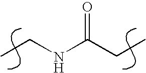 | mea2 |
| 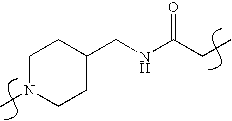 | pipeme2 |
| 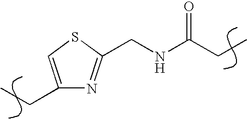 | me42thiaz2 |
| 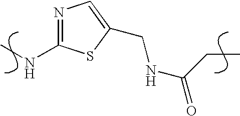 | a23thima2 |
| 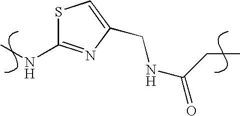 | a24thima2 |

The bond to the structural unit L should be understood as meaning a double bond in the compound where L=as.

| G′= | Abbreviation |
|---|---|
| 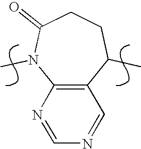 | 24pym |
| 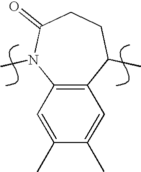 | dmeph |
| 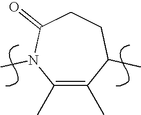 | dm |
| 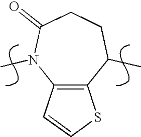 | thoph |
| 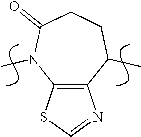 | thaph |
| 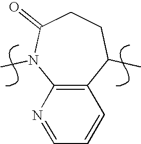 | 2pyph |
| 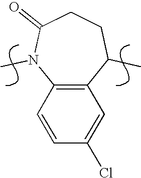 | 4clph |
| 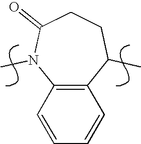 | phen |
| 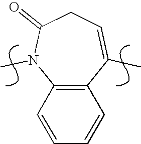 | dbph |
| 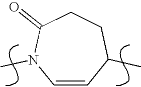 | hdb |
| 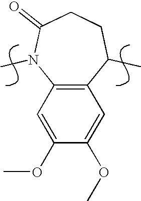 | dmeoph |
| 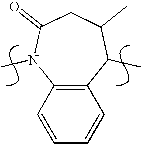 | meph |
| 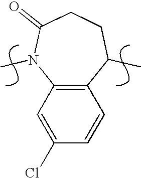 | 3clph |
| 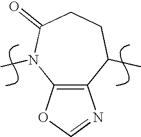 | oxph |
| 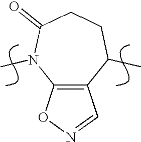 | ioph |
| 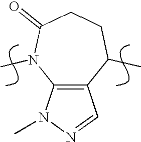 | yrph |
| 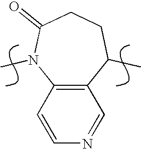 | 4pyph |
| 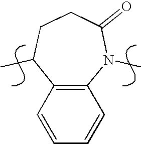 | reph |

| L= | Abbreviation |
|---|---|
| 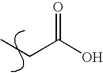 | es |
| 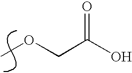 | gs |
| 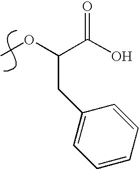 | pms |
| 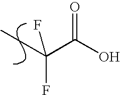 | f2es |
| 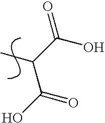 | mals |
| 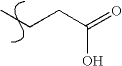 | ps |
| 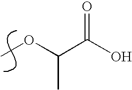 | ms |
| 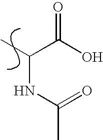 | nes |
| 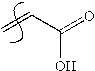 | as |

The compounds of the general formula I and thus also the compounds of the formula I′ as well as the starting substances used for their preparation can be prepared by methods of organic chemistry known to the person skilled in the art, such as are described in standard works such as Houben-Weyl, “Methoden der Organischen Chemie” [Methods of Organic Chemistry], Thieme-verlag, Stuttgart, or March “Advanced Organic Chemistry”, 4^{th} Edition, Wiley & Sons. Further preparation methods are also described in R. Larock, “Comprehensive Organic Transformations”, Weinheim 1989, in particular the preparation of alkenes, alkynes, halides, amines, ethers, alcohols, phenols, aldehydes, ketones, nitrites, carboxylic acids, esters, amides and acid chlorides.

The synthesis of compounds of the formula I can be carried out either according to the “classical” method in solution or on a polymeric support, in each case reaction conditions as are known and suitable for the respective reactions being used. Use can also be made in this case of variants which are known per se but not mentioned here.

The general synthesis of compounds of the formula I is described in schemes 1-7. If not stated otherwise, all starting materials and reagents are commercially available, or can be prepared from commercially obtainable precursors by customary methods.

Fused 2,3,4,5-tetrahydro-1H-azepinediones of type II are known and can be prepared by known methods, e.g. starting from anthranilic acid esters or the corresponding heterocyclic analogs via Dieckmann condensation and subsequent decarboxylation, as is described in the following publications: J. Am. Chem. Soc. 80, 1958, 2172-2178; J. Chem. Soc. 1959, 3111; J. Chem. Soc. 1934, 1326; Arch. Pharm. 324, 1991, 579-581. The preparation of 3,4-dihydro-1H-azepine-2,5-dione is described in Heterocycles 8, 1977, 345-350.

The conversion into compounds of the type III is generally carried out by methods known to the person skilled in the art, such as are described, for example, in Larock, “Comprehensive Organic Transformations”, Weinheim 1989, pp. 167ff, although methods which are not mentioned here can also be used. Preferably, compounds of the general formula III can be prepared by reaction of the ketones II with a phosphonic ester of the general formula (EtO)₂P(═O)(X_{L})ₐ(CR_{L}¹R_{L}²)_{b}—COOSG1 in the presence of a base.

The reaction preferably takes place in a polar aprotic solvent, such as tetrahydrofuran, dioxane; dimethylformamide (DMF), dimethylacetamide or acetamide; dimethyl sulfoxide, sulfolane; N-methylpyrrolidone, 1,3-dimethyltetrahydro-2(1H)-pyrimidinone (DMPU), 1,3-dimethyl-2-imidazolidinone; in a temperature range depending on the nature of the solvent used from −40° C. up to the boiling point of the corresponding solvent.

The base used can be an alkali metal or alkaline earth metal hydride such as sodium hydride, potassium hydride or calcium hydride, a carbonate such as alkali metal carbonate, e.g. sodium carbonate or potassium carbonate, an alkali metal or alkaline earth metal hydroxide such as sodium hydroxide or potassium hydroxide, an alkoxide such as sodium methoxide, potassium tert-butoxide, an organometallic compound such as butyllithium or alkali metal amides such lithium diisopropylamide, or lithium, sodium or potassium bis(trimethylsilyl)amide.

The reaction to give IV is carried out by hydrogenation of the double bond under standard conditions. Use can also be made here of variants which are known per se but not mentioned. Preferably, the hydrogenation is carried out in the presence of a noble metal catalyst, such as Pd on activated carbon, Pt, PtO₂, Rh on Al₂O₃ in an inert solvent at a temperature of 0-150° C. and a pressure of 1-200 bar; the addition of an acid such as acetic acid or hydrochloric acid can be advantageous. Hydrogenation in the presence of 5-10% Pd on activated carbon is particularly preferred.

Solvents which can be used are all customary inert solvents, such as hydrocarbons such as hexane, heptane, petroleum ether, toluene, benzene or xylene; chlorinated hydrocarbons such as trichloroethylene, 1,2-dichloroethane, carbon tetrachloride, chloroform, dichloromethane; alcohols such as methanol, ethanol, isopropanol, n-propanol, n-butanol or tert-butanol; ethers such as diethyl ether, methyl tert-butyl ether, diisopropyl ether, tetrahydrofuran, dioxane; glycol ethers such as ethylene glycol monomethyl ether or monoethyl ether, ethylene glycol dimethyl ether; ketones such as acetone, butanone; amides such as dimethylformamide (DMF), dimethylacetamide or acetamide; sulfoxides such as dimethyl sulfoxide, sulfolane; pyridine, N-methylpyrrolidone, 1,3-dimethyltetrahydro-2(1H)-pyrimidinone (DMPU), 1,3-dimethyl-2-imidazolidinone, water or mixtures of the solvents mentioned.

Compounds of type V are prepared by reaction with compounds of the general formula A-E-X¹ (VI), the radical X¹ being a customary leaving group, for example halogen such as chlorine, bromine, iodine or aryl- or alkylsulfonyl optionally substituted by halogen, alkyl or haloalkyl, such as toluenesulfonyl, trifluoromethanesulfonyl and methylsulfonyl or another equivalent leaving group. The reaction preferably takes place in an inert solvent (such as previously described) with addition of a suitable base, i.e. of a base which brings about deprotonation of the intermediate IV, in a temperature range from −40° C. up to the boiling point of the corresponding solvent.

The base used can be an alkali metal or alkaline earth metal hydride such as sodium hydride, potassium hydride or calcium hydride, a carbonate such as alkali metal carbonate, e.g. sodium or potassium carbonate, an alkali metal or alkaline earth metal hydroxide such as sodium hydroxide or potassium hydroxide, an alkoxide such as sodium methoxide, potassium tert-butoxide, an organometallic compound such as butyllithium or alkali metal amides such as lithium diisopropylamide, lithium bis(trimethylsilyl)amide, sodium bis(trimethylsilyl)amide or potassium bis(trimethylsilyl)amide.

Removal of the protective group SG1 according to standard conditions (see below) leads to the compounds of the general formula I. If SG1 is C₁-C₄-alkyl or benzyl, the compounds of the general formula V correspond directly to the compounds of type I.

Alternatively to this synthesis strategy, compounds of type I can also be prepared via VII as an intermediate, here too reaction conditions being used such as are known to the person skilled in the art and described in standard works. Compound VII is prepared by reaction of compounds of type IV having radio of the general formula D_{E}-E-X² (VIII) under reaction conditions such as have already been described for the preparation of V from IV and VI. X² here is a suitable leaving group, as has already been described for X¹, and D_{E} is CN, N₃ or a protected amino or acid function of the general formula NSG3 or COOSG2. The synthesis of the fragments D_{E}-E or A-E is carried out—depending on the actual structure of E—by removal of the protective groups and coupling of the remaining fragments according to standard methods, e.g. amide couplings. The introduction of A is then carried out analogously to the reactions described in schemes 3-7.

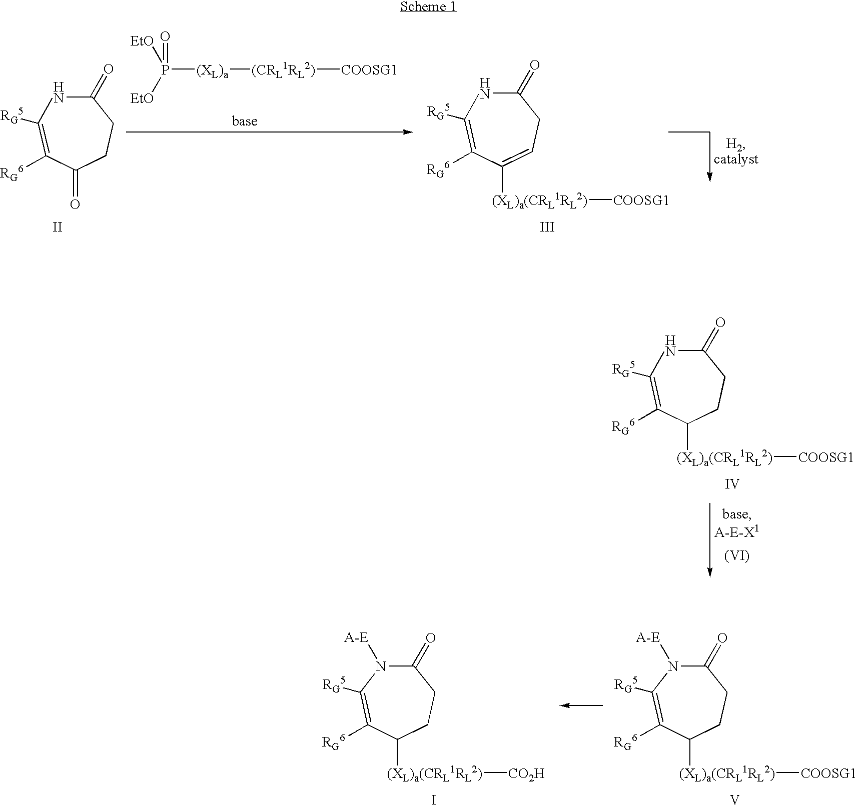

Compounds of the formula I in which W_{G} in structural element G is a structural element of the formula I_{WG}¹ can be prepared according to scheme 2.

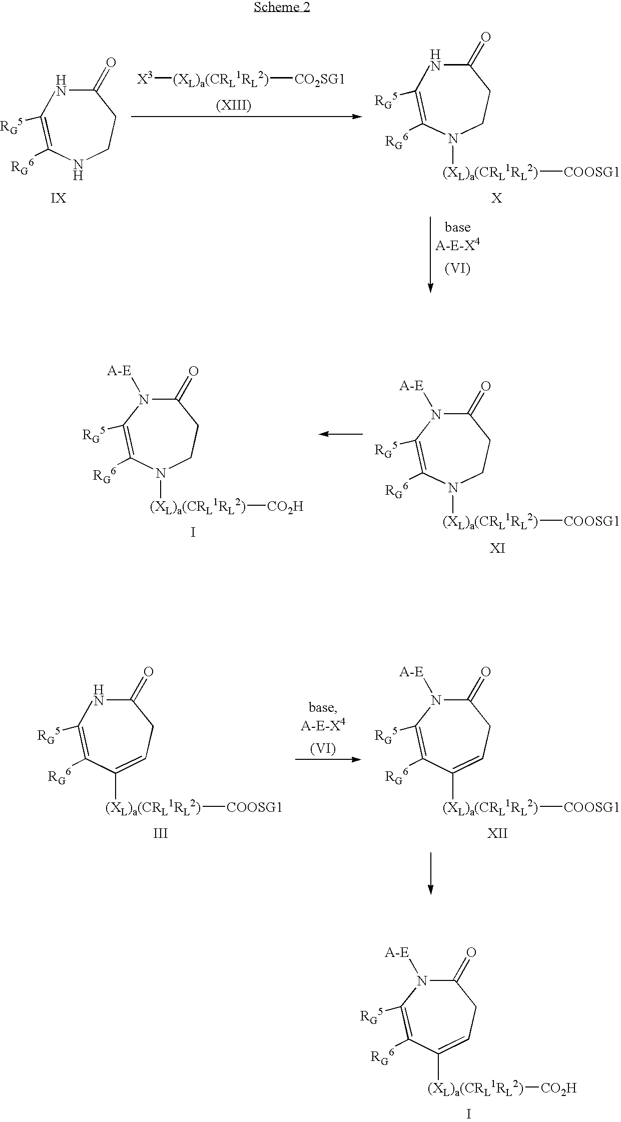

The starting point of the synthesis is compounds of type IX, which are either known or are accessible by methods known to the person skilled in the art, such as are described, for example, in J. Am. Chem. Soc. 71, 1949, 1985. Alkylation with a compound of the general formula XIII (X³, X⁴=a customary leaving group) under customary reaction conditions leads to X. The further reactions to give I then proceed analogously to scheme 1 via compounds of type XI.

In the case in which W_{G} in structural element G is a structural element of the formula I_{WG}³, compounds of type III can be converted into compounds of type XII and then into I analogously to the preparation of V (scheme 2).

The coupling of the individual fragments and the removal of the protective groups can be carried out according to known processes (see Larock, “Comprehensive Organic Transformations; protective groups: Greene, T., “Protective Groups in Organic Synthesis”, New York 1991), in the case of amide bonds also analogously to the methods of peptide synthesis, such as are described in standard works, e.g. in Bodanszky “The Practice of Peptide Synthesis”, 2^{nd} Edition, Springer-Verlag 1994, and Bodanszky “Principles of Peptide Synthesis”, Springer-Verlag 1984. A general survey of the customary methods for peptide synthesis and a listing of suitable reagents can furthermore be found in NOVABIOCHEM 1999 “Catalog and Peptide Synthesis Handbook”.

The amide couplings mentioned can be carried out with the aid of customary coupling reagents using suitably protected amino and carboxylic acid derivatives. Another method consists in the use of preactivated carboxylic acid derivatives, preferably of carboxylic acid halides, symmetrical or mixed anhydrides or so-called active esters, which are customarily used for the acylation of amines. These activated carboxylic acid derivatives can also be prepared in situ.

As a rule, the couplings can be carried out in inert solvents in the presence of an acid-binding agent, preferably of an organic base such as triethylamine, pyridine, diisopropylethylamine, N-methylmorpholine, quinoline; the addition of an alkali metal or alkaline earth metal hydroxide, carbonate or hydrogencarbonate or of another salt of a weak acid of the alkali metals or alkaline earth metals, preferably of potassium, sodium, calcium or cesium, can also be favorable.

Depending on the conditions used, the reaction time is between minutes and 14 days, the reaction temperature between −40° C. and 140° C., preferably between −20° C. and 100° C.

Suitable inert solvents are, for example, hydrocarbons such as hexane, heptane, petroleum ether, toluene, benzene or xylene; chlorinated hydrocarbons such as trichloroethylene, 1,2-dichloroethane, carbon tetrachloride, chloroform, dichloromethane; alcohols such as methanol, ethanol, isopropanol, n-propanol, n-butanol or tert-butanol; ethers such as diethyl ether, methyl tert-butyl ether, diisopropyl ether, tetrahydrofuran, dioxane; glycol ethers such as ethylene glycol monomethyl ether or monoethyl ether, ethylene glycol dimethyl ether; ketones such as acetone, butanone; amides such as dimethylformamide (DMF), dimethylacetamide or acetamide; nitriles such as acetonitrile; sulfoxides such as dimethyl sulfoxide, sulfolane; N-methylpyrrolidone, 1,3-dimethyltetrahydro-2(1H)-pyrimidinone (DMPU), 1,3-dimethyl-2-imidazolidinone, nitro compounds such as nitromethane or nitrobenzene; esters such as ethyl acetate; water; or mixtures of the solvents mentioned.

Protective groups SG which can be used are all protective groups which are known from peptide synthesis and customary to the person skilled in the art, such as are also described in the abovementioned standard works. The protective groups in the compounds of the formulae V, VII, XI and XII are likewise removed according to conditions such as are known to the person skilled in the art and are described by Greene and Wuts in “Protective Groups in organic Synthesis”, 2^{nd} Edition, Wiley & Sons, 1991.

Protective groups such as SG3 are so-called N-terminal amino protective groups; Boc, Fmoc, benzyloxycarbonyl (Z), acetyl, Mtr are preferred here.

SG1 and SG2 are acid protective groups; C₁-C₄-alkyl is preferred here, such as methyl, ethyl, tert-butyl, or alternatively benzyl or trityl, or alternatively polymer-bonded protective groups in the form of the commercially available polystyrene resins such as 2-chlorotrityl chloride resin or Wang resin (Bachem, Novabiochem).

Acid-labile protective groups (e.g. Boc, tert-butyl, Mtr, trityl) can be removed—depending on the protective group used—using organic acids such as trifluoroacetic acid (TFA), trichloroacetic acid, perchloric acid, trifluoroethanol; but also inorganic acids such as hydrochloric acid or sulfuric acid, sulfonic acids such as benzene- or p-toluenesulfonic acid, the acids generally being employed in an excess. HCl or TFA is preferably used. In the case of trityl, the addition of thiols such as thioanisole or thiophenol can be advantageous. The presence of an additional inert solvent is possible, but not always necessary. Suitable inert solvents are preferably organic solvents, for example carboxylic acids such as acetic acid; ethers such as THF or dioxane; amides such as DMF or dimethylacetamide; halogenated hydrocarbons such as dichloromethane; alcohols such as methanol, isopropanol; or water. Suitable solvents are also mixtures of those mentioned. The reaction temperature for these reactions is between −10° C. and 50° C.; the reaction is preferably carried out in a range between 0° C. and 30° C.

Base-labile protective groups such as Fmoc are cleaved by treatment with organic amines such as dimethylamine, diethylamine, morpholine or piperidine as 5-50% solutions in CH₂Cl₂ or DMF. The reaction temperature for these reactions is between −10° C. and 50° C.; the reaction is preferably carried out in a range between 0° C. and 30° C.

Acid protective groups such as methyl or ethyl are preferably cleaved by basic hydrolysis in an inert solvent. The bases used are preferably alkali metal or alkaline earth metal hydroxides, preferably NaOH, KOH or LiOH; the solvents used are all customary inert solvents, such as hydrocarbons such as hexane, heptane, petroleum ether, toluene, benzene or xylene; chlorinated hydrocarbons such as trichloroethylene, 1,2-dichloroethane, carbon tetrachloride, chloroform, dichloromethane; alcohols such as methanol, ethanol, isopropanol, n-propanol, n-butanol or tert-butanol; ethers such as diethyl ether, methyltert-butyl ether, diisopropyl-ether, tetrahydrofuran, dioxane; glycol ethers such as ethylene glycol monomethyl ether or monoethyl ether, ethylene glycol dimethyl ether; ketones such as acetone, butanone; amides such as dimethylformamide (DMF), dimethylacetamide or acetamide; nitriles such as acetonitrile; sulfoxides such as dimethyl sulfoxide, sulfolane; N-methylpyrrolidone, 1,3-dimethyltetrahydro-2(1H)-pyrimidinone (DMPU), 1,3-dimethyl-2-imidazolidinone; nitro compounds such as nitromethane or nitrobenzene; water or mixtures of the solvents mentioned. The addition of a phase-transfer catalyst—depending on the solvent or solvent mixture used—may be advantageous. The reaction temperature for these reactions is generally between −10° C. and 100° C.

Hydrogenolytically removable protective groups such as benzyloxycarbonyl (Z) or benzyl can be removed, for example, by hydrogenolysis in the presence of a catalyst (e.g. of a noble metal catalyst on activated carbon as support). Suitable solvents are those mentioned above, in particular alcohols such as methanol, ethanol; amides such as DMF or dimethylacetamide; esters such as ethyl acetate. As a rule, the hydrogenolysis is carried out at a pressure of 1-200 bar and temperatures between 0° and 100° C.; the addition of an acid such as acetic acid or hydrochloric acid may be advantageous. The catalyst used is preferably 5-10% Pd on activated carbon.

The synthesis of structural units of type E is generally carried out by methods known to the person skilled in the art; the structural units used are either commercially available or accessible by methods known from the literature. The synthesis of some of these structural units is described by way of example in the experimental section.

In the case in which the fragments Q_{E} or X_{E} contained in the compounds of type VI and VIII are a hetaryl radical, the structural units used are either commercially available or accessible by methods known to the person-skilled in the art. A multiplicity of preparation methods are described in detail in Houben-Weyl's “Methoden der organischen Chemie” (Vol. E6: furans, thiophenes, pyrroles, indoles, benzothiophenes, -furans, -pyrroles; Vol. E7: quinolines, pyridines, Vol. E8: isoxazoles, oxazoles, thiazoles, pyrazoles, imidazoles and their benzo-fused representatives, as well as oxadiazoles, thiadiazoles and triazoles; Vol. E9: pyridazines, pyrimidines, triazines, azepines and their benzo-fused representatives as well as purines). The linkage of these fragments to E, depending on the structure of E, can also take place via the amino or acid function according to methods which are known to the person skilled in the art.

Structures of the general formula A-E-D_{E} are synthesized according to methods known to the person skilled in the art, which are described, for example, in WO 97/08145. Examples of these are the conversion of compounds of the general formula:

HNR_{E}¹¹E_{A1}-D_{E} (XIV)

NC-E_{A2}-D_{E} (XV)

into compounds of the general formula:

A-HNR_{E}¹¹-E_{A1}-D_{E} (XVI)

A-E-D_{E} (XVII)

The groups E_{A1} and E_{A} in the formulae XIV-XVIII represent structural fragments which, after an appropriate modification (e.g. reaction with suitable reagents or coupling with appropriate structural units) as a whole form the structural fragment A-E. These structural units can then be reacted either directly—in the case of the corresponding free amines or carboxylic acids—or after removal of the protective groups—to give compounds of the general formula I (scheme 1 and 2). In principle, A, however, can also be introduced into compounds of the type IV, as described in scheme 1, where the reaction conditions mentioned can be used just as variants which are not described here.

In schemes 3-7, a number of the methods for the introduction of A are described by way of example, in each case reaction conditions being used which are known and suitable for the respective reactions. Use can also be made in this case of variants which are known per se but not mentioned here.

Ureas and thioureas (AE-1 to AE-3) can be prepared by customary methods of organic chemistry, e.g. by reaction of an isocyanate or of an isothiocyanate with an amine, if appropriate in an inert solvent with heating (Houben-Weyl Volume VIII, 157ff.) (scheme 3):

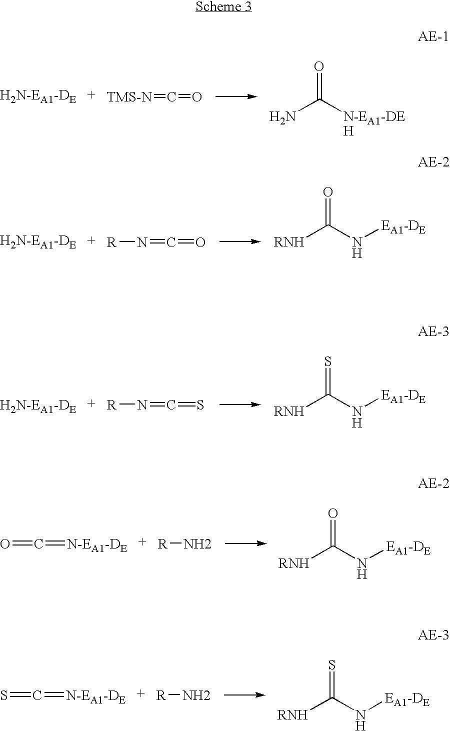

Scheme 4 shows, by way of example, the preparation of compounds of the type AE-4, as is described, for example, by Blakemoore et al. in Eur. J. Med. Chem. 1987 (22) 2, 91-100, or Misra et al. in Bioorg. Ned. Chem. Lett. 1994 4 (18), 2165-2170.

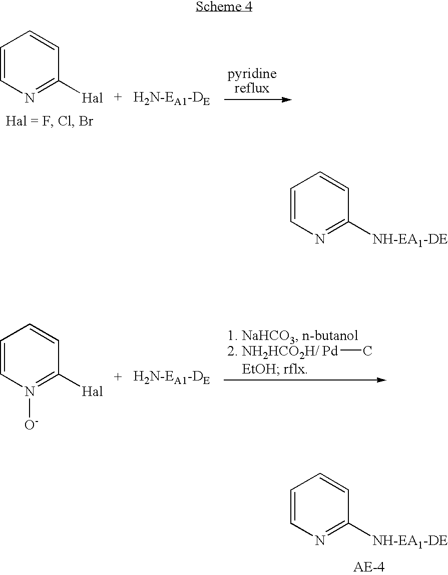

Unsubstituted or cyclic guanidine derivatives of the general formulae AE-5 and AE-6 can be prepared by means of commercially available or simply accessible reagents, such as are described, for example, in Synlett 1990, 745, J. Org. Chem. 1992, 57, 2497, Bioorg. Ned. Chem. 1996, 6, 1185-1208; Bioorg. Med. Chem. 1998, 1185, or Synth. Comm. 1998, 28, 741-746 (scheme 5).

The preparation of compounds of the general formula AE-7 can be carried out analogously to U.S. Pat. No. 3,202,660, compounds of the formulae AE-9, AE-10, AE-11 and AE-12 analogously to WO 97/08145. Compounds of the formula AE-8 can be prepared, as shown in scheme 6, e.g. according to the method described by Perkins et al., Tetrahedron Lett. 1999, 40, 1103-1106. Scheme 5 gives a survey of the synthesis of the compounds mentioned, the circle in AE-8 representing a fused cycle, such as aryl or hetaryl.

Compounds of the general formula AE-13 can be prepared analogously to Froeyen et al., Phosphorus Sulfur silicon Relat. Elem. 1991, 63, 283-293; AE-14 analogously to Yoneda et al., Heterocycles 1998, 15 N′-1, Spec. Issue, 341-344 (scheme 6). The preparation of corresponding compounds can also be carried out analogously to WO 97/36859.

Compounds of the general formula AE-15 can be prepared according to Synthesis 1981, 963-965 or Synth. Comm. 1997, 27 (15), 2701-2707; AE-16 analogously to J. Org. Chem. 1991, 56 (6), 2260-2262 (scheme 7), the circle being a fused cycle, such as aryl, hetaryl or cycloalkyl.

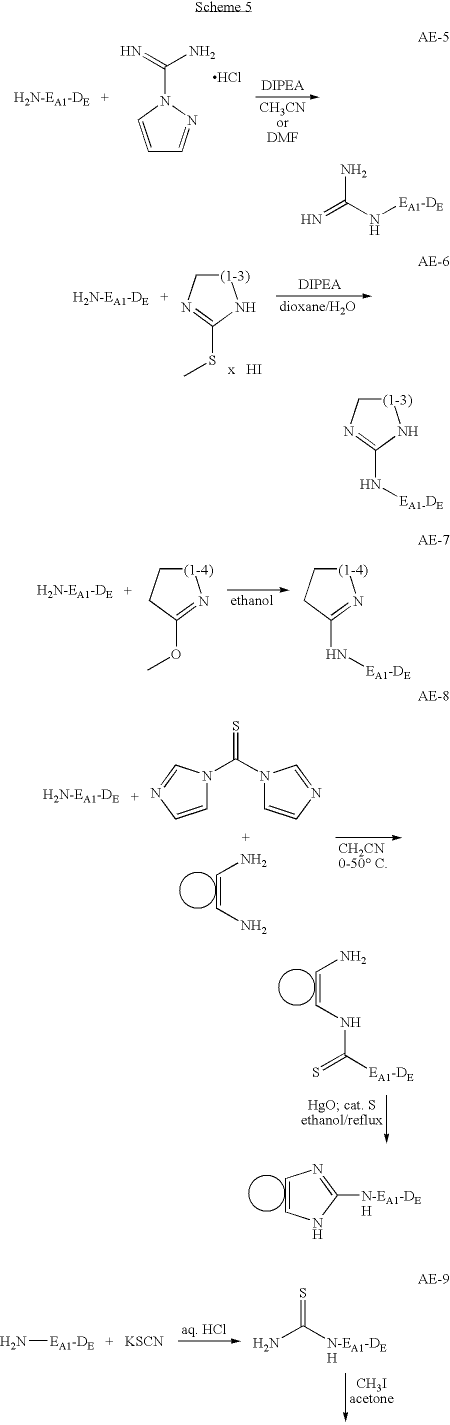

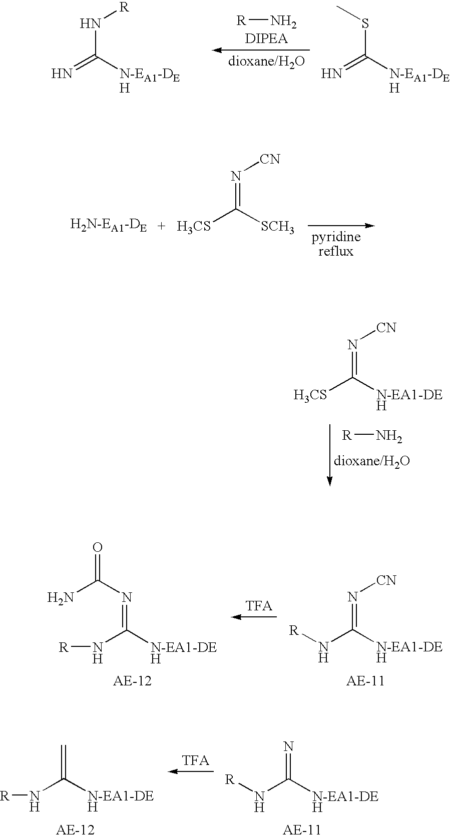

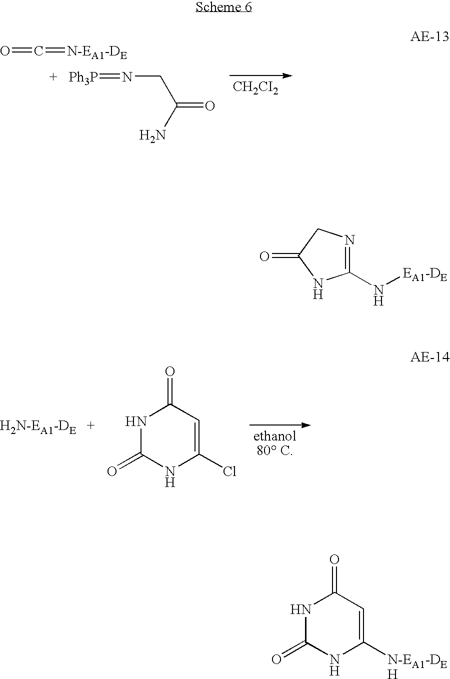

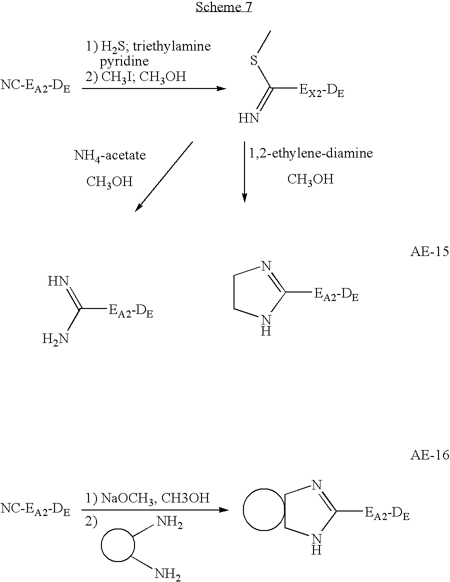

The invention further relates to pharmaceutical preparations, comprising at least one compound of the formula I′ in addition to the customary pharmaceutical excipients.

The compounds according to the invention can be administered orally or parenterally (subcutaneously, intravenously, intramuscularly, intraperitoneally) in the customary manner. Administration can also be carried out through the nasopharynx using vapors or sprays. Further, the compounds according to the invention can be introduced by direct contact with the affected tissue.

The dose depends on the age, condition and weight of the patient and on the manner of administration. As a rule, the daily dose of active compound is between approximately 0.5 and 50 mg/kg of body weight in the case of oral administration and between approximately 0.1 and 10 mg/kg of body weight in the case of parenteral administration.

The novel compounds can be administered in solid or liquid form in the customary pharmaceutical administration forms, e.g. as tablets, film-coated tablets, capsules, powders, granules, coated tablets, suppositories, solutions, ointments, creams or sprays. These are prepared in a customary manner. The active compounds can in this case be processed using the customary pharmaceutical excipients such as tablet binders, fillers, preservatives, tablet disintegrants, flow regulators, plasticizers, wetting agents, dispersants, emulsifiers, solvents, release-delaying agents, antioxidants and/or propellants (cf. H. Sucker et al.: Pharmazeutische Technologie, Thieme-Verlag, Stuttgart, 1991). The administration forms thus obtained normally contain the active compound in an amount from 0.1 to 90% by weight.

The invention further relates to the use of compounds of the formula I′ for the production of drugs for treating illnesses. The compounds of the formula I′ can be used for treating human and animal illnesses. The compounds of the formula I′ which represent the novel compounds of the formula I bind, as mentioned above, to integrin receptors. They are therefore suitable, as mentioned above, preferably as integrin receptor ligands and for the production of drugs for treating illnesses in which an integrin receptor is involved, in particular for the treatment of illnesses in which the interaction between integrins and their natural ligands is dysregulated, i.e. excessive or reduced, as described above.

Advantageously, the compounds of the formula I, preferably the compounds of the formula I′, can be administered in combination with at least one further compound in order to achieve an improved curative action in a number of indications. These further compounds can have the same or a different mechanism of action as/from the compounds of the formula I.

In addition to the compounds of the formula I, preferably in addition to the compounds of the formula I′ and the customary pharmaceutical excipients, the pharmaceutical preparations can therefore contain at least one further compound, depending on the indication, in each case selected from one of the 10 groups below.

### Group 1:

inhibitors of blood platelelet adhesion, activation or aggregation, such as acetylsalicylic acid, lysine acetylsalicylate, piracetam, dipyridamol, abciximab, thromboxane antagonists, fibrinogen antagonists, such as tirofiban, or inhibitors of ADP-induced aggregation such as ticlopidine or clopidogrel,
anticoagulants which prevent thrombin activity or formation, such as inhibitors of IIa, Xa, XIa, IXa or VIIa,
antagonists of blood platelet-activating compounds and selectin antagonists
for the treatment of blood platelet-mediated vascular occlusion or thrombosis, or

### Group 2:

inhibitors of blood platelet activation or aggregation, such as GPIIb/IIIa antagonists, thrombin or factor Xa inhibitors or ADP receptor antagonists,
serine protease inhibitors,
fibrinogen-lowering compounds,
selectin antagonists,
antagonists of ICAM-1 or VCAM-1
inhibitors of leukocyte adhesion
inhibitors of vessel wall transmigration,
fibrinolysis-modulating compounds, such as streptokinase, tPA,
plasminogen-activating stimulants, TAFI inhibitors, XIa inhibitors or PAI-1 antagonists,
inhibitors of complement factors,
endothelin receptor antagonists,
tyrosine kinase inhibitors,
antioxidants and
interleukin 8 antagonists
for the treatment of myocardial infarct or stroke, or

### Group 3:

endothelin antagonists,
ACE inhibitors,
angiotensin receptor antagonists,
endopeptidase inhibitors,
beta-blockers,
calcium channel antagonists,
phosphodiesterase inhibitors and
caspase inhibitors
for the treatment of congestive heart failure, or

### Group 4:

thrombin inhibitors,
inhibitors of factor Xa,
inhibitors of the coagulation pathway which leads to thrombin formation, such as heparin or low-molecular weight heparins, inhibitors of blood platelet adhesion, activation or aggregation, such as GPIIb-IIIa antagonists or antagonists of the blood platelet adhesion and activation mediated by vWF or GPIb,
endothelin receptor antagonists,
nitrogen oxide synthase inhibitors,
CD44 antagonists,
selectin antagonists,
MCP-1 antagonists,
inhibitors of signal transduction in proliferating cells, antagonists of the cell response mediated by EGF, PDGF, VEGF or bFGF and
antioxidants
for the treatment of restenosis after vascular injury or stent implantation, or

### Group 5:

antagonists of the cell response mediated by EGF, PDGF, VEGF or bFGF,
heparin or low-molecular weight heparins or further GAGs,
inhibitors of MMPs,
selectin antagonists,
endothelin antagonists,
ACE inhibitors,
angiotensin receptor antagonists and
glycosylation inhibitors or AGE formation inhibitors or AGE breakers and antagonists of their receptors, such as RAGE,
for the treatment of diabetic angiopathies, or

### Group 6:

lipid-lowering compounds,
selectin antagonists,
antagonists of ICAM-1 or VCAM-1
heparin or low-molecular weight heparins or further GAGs,
inhibitors of MMPs,
endothelin antagonists,
apolipoprotein A1 antagonists,
cholesterol antagonists,
HMG CoA reductase inhibitors,
ACAT inhibitors,
ACE inhibitors,
angiotensin receptor antagonists,
tyrosine kinase inhibitors,
protein kinase C inhibitors,
calcium channel antagonists,
LDL receptor function stimulants,
antioxidants
LCAT mimetics and
free radical scavengers
for the treatment of atherosclerosis, or

### Group 7:

cytostatic or antineoplastic compounds,
compounds which inhibit proliferation, such as kinase inhibitors and
heparin or low-molecular weight heparins or further GAGS
for the treatment of cancer, preferably for the inhibition of tumor growth or metastasis, or

### Group 8:

compounds for antiresorptive therapy,
compounds for hormone exchange therapy, such as estrogen or progesterone antagonists,
recombinant human growth hormone,
bisphosphonates, such as alendronates
compounds for calcitonin therapy,
calcitonin stimulants,
calcium channel antagonists,
bone formation stimulants, such as growth factor antagonists, interleukin-6 antagonists and
Src tyrosine kinase inhibitors
for the treatment of osteoporosis, or

### Group 9:

TNF antagonists,
antagonists of VLA-4 or VCAM-1,
antagonists of LFA-1, Mac-1 or ICAMs,
complement inhibitors,
immunosuppressants,
interleukin-1, -5 or -8 antagonists and
dihydrofolate reductase inhibitors
for the treatment of rheumatoid arthritis, or

### Group 10:

collagenase,
PDGF antagonists and

### MMPs

for improved wound healing.

A pharmaceutical preparation comprising at least one compound of the formula I, preferably comprising at least one compound of the formula I′, if appropriate pharmaceutical excipients and at least one further compound, depending on the indication, in each case selected from one of the above groups, is understood as meaning a combined administration of at least one of the compounds of the formula I, preferably of one of the compounds of the formula I′, with at least one further compound in each case selected from one of the groups described above and, if appropriate, pharmaceutical excipients.

Combined administration can be carried out by means of a substance mixture comprising at least one compound of the formula I, preferably of the formula I′, if appropriate pharmaceutical excipients and at least one further compound, depending on the indication, in each case selected from one of the above groups, but also spatially and/or chronologically separate.

In the case of the spatially and/or chronologically separate administration, the administration of the components of the pharmaceutical preparation, the compounds of the formula I, preferably of the formula I′ and the compounds selected from one of the abovementioned groups, takes place spatially and/or chronologically separately.

For the treatment of restenosis after vascular injury or stenting, the administrations of the compounds of the formula I, preferably of the formula I′, can be carried out locally at the affected sites, on their own or in combination with at least one compound selected from group 4. It may also be advantageous to coat the stents with these compounds.

For the treatment of osteoporosis, it may be advantageous to carry out the administration of the compounds of the formula I, preferably of the formula I′, in combination with an antiresorptive or hormone exchange therapy.

The invention accordingly relates to the use of the abovementioned pharmaceutical preparations for the production of drugs for treating illnesses.

In a preferred embodiment, the invention relates to the use of the abovementioned combined pharmaceutical preparations for the production of drugs for treating

blood platelet-mediated vascular occlusion or thrombosis
when using compounds of group 1,
myocardial infarct or stroke
when using compounds of group 2,
congestive heart failure
when using compounds of group 3,
restenosis after vascular injury or stent implantation
when using compounds of group 4,
diabetic angiopathies
when using compounds of group 5,
atherosclerosis
when using compounds of group 6,
cancer
when using compounds of group 7,
osteoporosis
when using compounds of group 8,
rheumatoid arthritis
when using compounds of group 9,
wound healing
when using compounds of group 10.

The following examples illustrate the invention, the selection of these examples being non-limiting.

### I. SYNTHESIS EXAMPLES

### I.A Precursors

### Example 1

### t-Butyl (2-oxo-2,3-dihydro-1H-1-benzazepin-5-yl)acetate (1)

22.3 g (80 mmol) of t-butyl diethylphosphonate (95%) were added dropwise at 0° C. to a suspension of 3.27 g of NaH (60%; deoiled) in ml of DMF. The mixture was stirred until a clear solution was formed and then 12.4 g (70.9 mmol) of 3,4-dihydro-1H-1-benzazepine-2,5-dione (preparation according to Arch. Pharm. 1991, 324, 579) in 90 ml of DMF were added dropwise at 0° C. The reaction mixture then remained standing at RT for about 3 days. For workup, the mixture was poured into 700 ml of cold 5% NaCl solution, and the resulting yellow precipitate was filtered off with suction and washed with H₂O. The moist residue was taken up in CH₂Cl₂, washed with 5% NaHCO₃ solution and dried over Na₂SO₄. The residue which remained after evaporation was treated with 150 ml of cyclohexane in the presence of heat, and after cooling, filtering off with suction and washing with n-hexane 17.5 g (90.5%) of white crystals remained; m.p.: 136-138° C.

### Example 2

### t-Butyl (2-oxo-2,3,4,5-tetrahydro-1H-1-benzazepin-5-yl)acetate (2)

A suspension of 3 g of 10% Pd/C in 50 ml of ethanol was prehydrogenated, then a solution of compound 1 (14.7 g; 53.8 mmol) in 125 ml of ethanol and 75 ml of dioxane was added, and the mixture was hydrogenated under standard conditions until the absorption of hydrogen was complete. After filtering off the catalyst with suction and washing it with ethanol, the filtrate was concentrated in vacuo, the oily residue was dissolved in diethyl ether and the crystallization commencing was completed by addition of n-hexane. After filtering off the precipitate with suction and washing it with n-hexane, 14.2 g (96%) of white crystals remained; m.p.: 101-103° C.

### Example 3

### [5-(2-t-Butoxy-2-oxoethyl)-2-oxo-2,3,4,5-tetrahydro-1H-benzazepin-5-yl]acetic acid (3)

a.) A solution of compound 2 (16.8 g; 61.1 mmol) in 60 ml of DMF was added dropwise at 10-20° C. to a suspension of 2.6 g of NaH (60%, deoiled) in 35 ml of DMF and the mixture was stirred until the appearance of an almost clear yellowish solution. t-Methyl bromoacetate (10 g; 63.4 mmol) was then added dropwise and the mixture was stirred overnight. For workup, the reaction mixture was poured into 400 ml of 5% cold NaCl solution and extracted 3× with 100 ml each of a diethyl ether/n-hexane mixture. The combined extracts were then washed with H₂O, 10% NaHCO₃ solution and NaCl solution, dried over Na₂SO₄, filtered and evaporated. The residual yellowish oil was reacted further without further purification; FAB-MS: 348 [M−H⁺].

b.) Crude product 3a was dissolved in 100 ml of dioxane and 65 ml of 1N NaOH were added dropwise at RT with stirring. After about 45′, the reaction mixture was adjusted to pH 7 using 1N KHSO₄ solution, the dioxane was largely distilled off in vacuo, and the residue was diluted with H₂O, adjusted to pH 9 using 1N NaOH and extracted 3× with diethyl ether. The aqueous phase was then rendered acidic using 1N KHSO₄ solution, the acid precipitating was extracted with a mixture of diethyl ether/n-hexane 4:1, and the organic phase was washed with H₂O, 1N NaOH solution and NaCl solution and dried over Na₂SO₄. Filtration and evaporation afforded an oily residue, which could be crystallized by treatment with diethyl ether/n-hexane 1:4 (water-saturated). Filtering with suction, washing with n-hexane and drying afforded 17.8 g (87.5%) of white crystals: m.p.: 117-119° C.

### Example 4

### N-[4-(Aminomethyl)phenyl]-1H-benzimidazole-2-amine (hydrochloride) (4)

a.) 20 g of tert-butyl-4-aminobenzyl carbamate (89.97 mmol)—dissolved in 100 ml of CH₃CN—were added dropwise at 0° C. to a solution of 24.5 g of thiocarbonyldiimidazole and 1.56 g of imidazole in 600 ml of CH₃CN and the mixture was stirred at RT overnight. 19.5 g of 1,2-phenylenediamine were then added and the mixture was again stirred at RT for 2 h. For workup, the reaction mixture was evaporated in vacuo, the residue was taken up in CH₂Cl₂, and the solution was washed 7× with 10% citric acid solution and 2× with satd. NaCl solution, dried over Na₂SO₄, filtered and concentrated. The crude product thus obtained (31.78 g; brown foam) was reacted directly without further purification; ESI-MS [M+H⁺]=373.15.

¹H-NMR (360 MHz, DMSO) δ ppm: 9.5 and 9.05 (each s, 1H), 7.45 (d, 2H), 7.35 (m, 1H), 7.20 (d, 1H), 7.15, 6.95, 6.75, 6.60 (each m, 1H), 4.85 (s, 2H), 4.10 (d, 2H), 1.35 (s, 9H).

b.) Crude product 4a was dissolved in 750 ml of ethanol together with 36.7 g of HgO (yellow) and 0.4 g of sulfur and heated to reflux for 2 h. The reaction mixture was then filtered twice through Celite and evaporated to dryness; 20.7 g, ESI-MS [M+H⁺]=339.15.

c.) 7 g of the crude product 4b were introduced into 70 ml of CH₂Cl₂, 35 ml of HCl in diethyl ether (satd. at 0° C.) were added and the mixture was stirred at RT for 2 h. The resulting precipitate was filtered off with suction, washed with CH₂Cl₂ and dried.

6.7 g of brown amorphous solid; ESI-MS [M+H⁺]=239.15

1H-NMR (360 MHz, DMSO) δ ppm: 11.6 (s broad, 1H), 8.4 (s broad, 3H), 8.25 (s broad, 1H), 7.65 and 7.55 (each d, 2H), 7.45 and 7.3 (each m, 2H), 4.19 (m, 2H).

### Example 5

### N-[5-(Aminomethyl)-1,3-thiazol-2-yl]guanidine (dihydrochloride) (5)

a.) 31 g (130 mmol) of 2-chloro-3-(1,3-dioxo-1,3-dihydro-2H-isoindol-2-yl)propanal (preparation according to THL 39 (1998), 8085-8088) and 15.4 g of amidinothiourea were heated at 110° C. for 75′ in 200 ml of n-butanol, then the mixture was evaporated and the residue was treated with CH₂Cl₂ and conc. NH₃. Evaporation of the organic phase, purification of the residue by chromatography on silica gel (CH₂Cl₂/CH₃OH 0-5%) and crystallization from acetone afforded 12.3 g of N-{5-[(1,3-dioxo-1,3-dihydro-2H-isoindol-2-yl)methyl]-1,3-thiazol-2-yl}guanidine.

b.) 1 g of 5a in 20 ml of CH₃OH was treated with 0.81 ml of hydrazine hydrate and stirred at RT for 2 h. The mixture was then cooled to 0° C., filtered, and the filtrate was concentrated and stirred with dilute HCl. This process was repeated a number of times, and the crude product obtained in this way was then stirred with ethanol; 0.92 g of white solid, ESI-MS [M+H⁺]=172.05.

### Example 6

### N-[4-(Aminomethyl)phenyl]-N′-benzylurea (6)

a.) 4-Aminobenzylamine (10.0 g, 81.85 mmol) in 150 ml CH₂Cl₂ was treated with triethylamine (6.8 g, 67.12 mmol) and then treated at 0° C. with di-t-butyl dicarbonate (18.6 g, 85.0 mmol). The mixture was stirred at 0° C. for 1 h and then at RT for 2 h. For workup, 150 ml of a 1%-aqueous citric acid solution were added, the phases were separated and the aqueous phase was reextracted 2 times with CH₂Cl₂ (150 ml). Fresh washing with H₂O, drying of the combined organic phases using Na₂SO₄ and evaporation afforded a solid, which was washed with stirring with a little diisopropyl ether, filtered off with suction and dried.

13.0 g; ESI-MS [M+H⁺-^{t}Bu]=167.05.

¹H-NMR (360 MHz, CDCl₃) δ (ppm): 7.04 (2H, d), 6.61 (2H, d), 4.78 (1H, s br.), 4.17 (2H, d), 3.67 (2H, s br.), 1.46 (9H, s).

b.) Benzyl isocyanate (2.40 g, 18.0 mmol) was added with ice-cooling to a solution of the protected amine 6a (4.0 g, 17.99 mmol) and triethylamine (1.82 g, 18.0 mmol) in 220 ml of toluene/DMF 10:1. The reaction mixture was stirred at RT overnight. It was possible to filter off some of the urea formed directly as a precipitate and dry it. The filtrate was washed 2× with H₂O, with dilute tartaric acid to pH 3 and again 2 times with H₂O to pH 5, and the organic phase was then dried and evaporated. Altogether, 6.0 g were thus obtained; ESI-MS [M+H⁺-^{t}Bu]=300.15.

c.) The urea 6b thus obtained was introduced in 90 ml of CH₂Cl₂, and TFA (2.24 g, 196.25 mmol)—dissolved in 90 ml of CH₂Cl₂—was added dropwise at 0° C. After 3 h, 1 ml of TFA was added again, then the mixture was stirred at RT overnight. After fresh addition of 1 ml of TFA, the mixture was stirred for a further 5 h, then poured onto ice water and extracted with ethyl acetate (2×50 ml). The water phase was rendered basic with 2N NaOH solution and extracted with CH₂Cl₂ (2×50 ml). The insoluble portion between the phases was filtered off and dried.

4 g; ESI-MS [2M+H⁺]=511.35

1H-NMR (200 MHz, DMSO) δ (ppm): 8.52 (1H, s), 7.39-7.07 (9H, m), 6.62 (1H, t), 4.27 (2H, d), 3.61 (2H, s).

### Example 7

### [4-(1H-Benzimidazol-2-yl)phenyl]methaneamine (hydrochloride) (7)

a.) Di(tert-butyl) 4-cyanobenzylimidodicarbonate (10 g, 30.08 mmol; preparation according to Synth. Comm. 28, 23, 1998, 4419ff) in 200 ml of pyridine was treated with 45 ml of triethylamine and saturated at 0° C. with H₂S for 1.5 h. The reaction mixture was allowed to stand at RT overnight and then evaporated. The residue thus obtained was then stirred with diethyl ether, filtered off with suction and dried (8.5 g).

b.) 6 g of the thioamide 7a (16.37 mmol) in 40 ml of dry CH₂Cl₂ were alkylated at RT overnight using 23.2 g of CH₃I and the mixture was then evaporated. The residue thus obtained was taken up in 40 ml of CH₃OH, 1.95 g of 1,2-phenylenediamine were added and it was again stirred overnight. Evaporating the reaction mixture and stirring the solid with n-pentane afforded 6.9 g of the desired benzimidazole.

M.p.: >170° C. (decomposition); ESI-MS: [M+H⁺]=424.25

c.) 1 g of the bis-Boc compound 7b was dissolved in 5 ml of CH₂Cl₂, 5 ml of TFA were added at 0° C. and the mixture was stirred at room temperature for 1 h. Evaporating the reaction mixture, treating with HCl in diethyl ether and stirring the isolated solid with diethyl ether afforded 0.6 g of the amine as the hydrochloride; ESI-MS: [M+H⁺]=224.05.

### Example 8

### N¹-(1H-Benzimidazol-2-yl)pentane-1,5-diamine (hydrochloride) (8)

Preparation was carried out analogously to the preparation of compound 4 starting from 7 g of N-Boc-1,5-diaminopentane hydrochloride (29.3 mmol). After reaction analogously to 4a, 10.3 g of N-Boc 5-{[(2-aminoanilino)carbothioyl]amino}pentane-1-amine were obtained; ESI-MS [M+H⁺]=353.25.

Cyclodesulfurization and subsequent removal of the Boc group with TFA afforded an oily crude product, which was taken up in CH₃OH and converted into the corresponding hydrochloride using 250 ml of ethereal HCl (saturated at 0° C.). Stirring the solid obtained with a mixture of CH₃OH/methyl t-butyl ether afforded 1.8 g of a reddish amorphous solid.

¹H-NMR (360 MHz, DMSO) δ ppm: 9.30 (t, 1H), 8.15 (s broad, 3H), 7.40 and 7.25 (each m, 2H), 3.35 (m, 2H superimposed with H₂O peak), 2.80 (m, 2H), 1.65 (m, 4H), 1.45 (m, 2H).

### Example 9

### N¹-(1H-Benzimidazol-2-yl)butane-1,4-diamine (trifluoroacetate) (9)

Preparation was carried out analogously to the preparation of compound 4 starting from 9.87 g of N-Boc-1,4-diaminobutane (52.3 mmol). After reaction analogously to 4a, 17.08 g of N-Boc 4-{[(2-aminoanilino)carbothioyl]amino}butane-1-amine were obtained; ESI-MS [M+H⁺]=338.99.

Subsequent cyclodesulfurization and Boc removal using TFA afforded a brown solid, which was stirred a number of times with n-pentane and then recrystallized from a mixture of CH₃OH/methyl t-butyl ether; 14.35 g, ESI-MS [M+H⁺]=205.15.

¹H-NMR (360 MHz, DMSO) δ ppm: 9.20 (t, 1H), 7.80 (s broad, 3H), 7.35 and 7.20 (each m, 2H), 3.40 (m, 2H partially superimposed with H₂O peak), 2.80 (m, 2H), 1.65 (m, 4H).

### Example 10

### trans-N-[(4-Aminocyclohexyl)methyl]-1H-benzimidazole-2-amine (dihydrochloride) (10)

Preparation was carried out analogously to compound 4 starting from 5.4 g of tert-butyl-4-(aminomethyl)cyclohexylamine carbamate (WO 9603374; Bioorg. Med. Chem. Lett. 1997, 7 (1), 67). After removal of the Boc group, 3.3 g of white dihydrochloride were obtained; FAB-MS [M+H⁺]: 245.

### Example 11

### trans-N-{[4-(Aminomethyl)cyclohexyl]-1H-benzimidazole-2-amine (dihydrochloride) (11)

Preparation was carried out analogously to compound 4 starting from 10 g of benzyl {4-[(tert-butoxycarbonyl)amino]cyclohexyl}-methylcarbamate (EP 669317) by removal of the Boc group using 4N HCl in dioxane, synthesis of the benzimidazole and subsequent hydrogenolysis. 3.6 g of white dihydrochloride were isolated; FAB-MS [M+H⁺]: 245.

### Example 12

### [6-(1H-Benzimidazol-2-yl)pyridin-3-yl]methaneamine (trifluoroacetate) (12)

a.) Preparation was carried out analogously to 7 starting from tert-butyl (6-cyanopyridin-3-yl)methylcarbamate (6.0 g, 25.72 mmol); crystallization of the crude product from ethanol afforded 5.15 g; ESI-MS [M+H⁺]=325.

b.) 0.55 g of the Boc-protected amine 12a in 10 ml of CH₂Cl₂ was treated with 5 ml of TFA and stirred at RT for 2 h. Evaporation of the reaction mixture afforded 0.95 g of a white solid; ESI-MS [M+H⁺]: 225.25.

### Example 13

### N-[4-(Aminomethyl)phenyl]-2-pyridineamine (13)

tert-Butyl 4-aminobenzylcarbamate (2 g; 9 mmol) was heated to reflux for 32 h with 8.74 g of 2-fluoropyridine. The reaction mixture was evaporated in vacuo and the residue obtained was stirred with n-pentane (1.9 g). The Boc group was cleaved using TFA, and the crude product obtained was precipitated from diethyl ether as the hydrochloride and then converted into the free-base (0.8 g) using NH₃; ESI-MS [M+H⁺]: 200.25.

### N-[4-(Aminomethyl)benzyl]-2-pyridineamine (14)

a.) 20 g 2-Aminopyridine were dissolved in 100 ml CH₃OH, adjusted to pH 6 with isopropanolic HCl and 36 g p-cyanobenzaldehyde were added. 9.35 g Sodium cyanoborohydride were added portionwise over one hour and stirred overnight. For workup, the suspension was evaporated, the residue taken up in 100 ml water and with KOH adjusted to pH>10. The watery phase was saturated with NaCl and extracted 3× with diethylether. The ether phase was washed after filtration of a precipitate 3× with a FeSO₄ solution, dried and evaporated. Purification of the residue by chromatography on silica gel (heptane/ethyl acetate 1:1) afforded 28.15 g 4-[2-pyridinyl-amino)methyl]benzonitrile.

b.) 10 g 4-[2-Pyridinyl-amino)methyl]benzonitrile were dissolved in 280 ml ammonia-alkali CH₃OH, 10 g Raney-nickel added and it was hydrogenated for 24 h. It was filtered, evaporated and the residue purified by chromatography on silica gel (ethyl acetate/ethyl alcohol 1:3).

5.18 g, ESI-MS: [M+H⁺]=214.

### [5-(1H-Benzimidazole-2-yl(thiene-2-yl]methaneamine (15)

Preparation was carried out starting from 5-(aminomethyl)thiophene-2-carbonitrile (preparation according to WO 95/23609), which was reacted to the respective Boc-derivative according to standard methods.

1.1 eq. Sodium methanolate solution was added to t-butyl-5-cyanothiene-2-ylcarbamate (25 g; 104.9 mmol) in 330 ml CH₃OH and stirred overnight at RT, then for 2 h at 40-50°. 18.95 g Phenylenediaminebihydrochloride were then added and again stirred at RT. For workup, water was added, the resulting precipitate filtered off and carefully dried. 19.6 yellow solid; ESI-MS: [M+H⁺]=330. The following cleavage of the Boc-group with TFA afforded a raw product, which was dissolved in water, 2× extracted with diethyl ether, the watery phase adjusted to pH 10-11 and then extracted 2× with ethyl acetate. The watery phase was saturated with NaCl and again extracted with ethyl acetate. The combined organic phases were dried and evaporated (6.3 g); ESI-MS [M+H⁺]=230.1.

### tert-Butyl-2-[4-(1H-benzimidazole-2-yl)phenyl]ethylcarbamate (16)

Preparation was carried out analogously to the preparation of [4-(1H-benzimidazole-2-yl)phenyl]methaneamine (hydrochloride) (7) starting from tert-butyl-2-(4-cyanophenyl)ethylcarbamate. The raw product obtained after reaction with H₂S, alkylation with CH₃I and reaction with 1,2-phenylenediamine was purified by chromatography on silica gel (CH₂Cl₂/CH₃OH 4-50%) (4.8 g); ESI-MS [M+H⁺]=338.15. The amine required for the further reaction was obtained by cleavage of the Boc-group with TFA (under standard conditions); the isolated TFA-salt was then directly utilized in the respective couplings.

### N-(Piperidine-4-ylmethyl)-1H-benzimidazole-2-amine (trifluoroacetate) (17)

a) A solution of tert-butyloxycarbonyl-4-(aminomethyl)-1-piperidine (5.39 g; 25 mmol) in 25 ml CH₃CN was added dropwise to 6.75 g thiocarbonyldiimidazole and 0.5 g imidazole in 100 ml CH₃CN at 0° C. and then stirred for 3 h at RT. 1,2-Phenylenediamine (5.5 g; 50.86 mmol) was then added and heated for about 1 h to 60° C. The solid obtained upon cooling was filtered off with suction and dried.

6.79 g; ESI-MS [M+H⁺-^{t}Bu]=309.15

b) tert-Butyoxycarbonyl-4-({[(2-aminoanilino)carbothioyl]amino}methyl)1-piperidine (5 g; 13.72 mmol), 5.94 g HgO (yellow) and 0.6 g sulfur in 150 ml ethyl alcohol were heated under reflux for 1 h. The mixture was filtered 2× over Celite, evaporated and the obtained raw product purified by chromatography on silica gel (CH₂Cl₂/CH₃OH 5-25%).

2.65 g; ESI-MS [M+H⁺]=331.25

¹H-NMR (360 MHz, DMSO) δ ppm: 7.15 and 6.9 (each m, 2H), 3.95 (d, 2H) 3.2 (m, 2H, 2.7 (br m; 2H), 1.8 (m, 1H), 1.7 (m, 2H), 1.35 (s, 9H), 1.05 (m, 2H).

c) tert-Butyloxycarbonyl-4-[(1H-benzimidazole-2-ylamino)methyl]-1-piperidine (2.65 g; 8.02 mmol) was treated with 10 ml TFA according to standard conditions. Evaporation and mixing the raw product with n-pentane afforded 2.39; ESI-MS [M+H⁺]=231.15.

¹H-NMR (360 MHz, DMSO δ ppm: 13.25 (s, 1H), 9.35 (m, 1H), 8.8 and 8.5 (each br s, 1H), 7.4 and 7.20 (each m, 2H), 3.3 (m, 4H), 2.85 (m, 2H), 1.9 (m, 3H), 1.35 (m, 2H).

### N-{[5-(Aminomethyl)thiene-3-yl]methyl}pyridine-2-amine (trifluoroacetate) (18)

a) A solution of tert-butyl-(4-cyanothiene-2-yl)methylcarbamate (7 g; 29.4 mmol) in 120 ml ethyl alcohol was saturated with NH₃ and then hydrogenated under standard conditions in the presence of Ra—Ni (9 g watery suspension; decanted with ethyl alcohol). Filtration of the reaction mixture, evaporation and chromatography of the obtained residue on silica gel (CH₂Cl₂/CH₃OH plus watery NH₃) afforded 4.4 g of the amine as a yellow oil.

b) 1.2. of the amine 18a (4.3 mmol), 06.g ethyldiisopropylamine and 15 g 2-fluoropyridine were heated for 20 h to reflux. The residue obtained after evaporation of the mixture was taken up in CH₂Cl₂, washed with 0.1n HCl and saturated NaCl solution, dried and again evaporated.

1 g; ESI-MS [M+H⁺]=320.15

c) 0.9 of the Boc-protected amine 18b were dissolved in 10 ml CH₂Cl₂, 5 ml TFA was added at 0° C. and it was stirred at room temperature for 1 h. Evaporation of the reaction mixture afforded 1.65 g of a brownish oil which was reacted directly without further purification (ESI-MS [M+H⁺]=220.05).

### 3-Amino-N-(1H-imidazole-2-yl)propaneamide (19)

a) Z-β-Alanine (10 g; 44.8 mmol) was dissolved in 200 ml DMF and 15.86 g (3.5 eq) N-methylmorpholine and 5.9 g (0.5 eq) 2-aminoimidazolesulfate were added. 7.87 g (1.3. eq) HOBt and 11.16 g (1.3 eq) N′-(dimethylaminopropyl)-N-ethylcarbodiimide were added at −10° C. and stirred for 1 h whilst to RT and then for 18 h. 150 ml diethyl ether were added whereupon a wide residue precipitated which was filtered with suction. The residue was washed with cold diethyl ether, suspended in ethyl acetate and 1n HCl was added up to an acid reaction. The watery solution was extracted 1× with ethyl acetate, the watery phase was then adjusted to a basic pH with 10% NaOH at 4° C. The resulting precipitate was filtered with suction and washed with water. 5.4 g; ESI-MS [M+H⁺]=289.05.

b) 5.3 g of the Z-compound 19a were suspended in 250 ml ethyl alcohol and 530 mg 10% Pd on activated carbon was added. It was hydrogenated with H₂ for 18 h at RT, then diluted with CH₃OH and the suspension was boiled up whereon the precipitate of the product disintegrated. Filtering and evaporation of the solution afforded 1.5 g; ESI-MS [M+H⁺]=155.05.

### N-[4-(Aminomethyl)phenyl]-4,5-dihydro-1H-imidazole-2-amine (hydrochloride) (20)

tert-Butyl-4-aminobenzylcarbamate (2 g; 9 mmol), 2.2 g ethyldiisopropylamine and 4.4 g (2-(3,5-dimethylpyrazole)-4,5-dihydroimidazole×HBr in 15 ml DMF were stirred at 110° C. After the reaction had completed, the mixture was evaporated, the residue taken up in ethyl acetate, each 2× washed with saturated NaHCO₃ and NaCl solution, dried and again evaporated. The basic watery phase was also evaporated, mixed with acetone, the precipitate filtered with suction and the mother liquor evaporated. The combined residues were purified by means of MPLC (silica gel: Fa. Bischoff Prontoprep 60-2540-C18E, 32 μm; solvent: CH₃CN/H₂O+0.1% acetic acid). 0.22 g; ESI-MS [M+H⁺]=291.15.

The product thus obtained was treated with 4n HCl in dioxane for 2 h at RT. The resulting precipitate was filtered with suction, washed with pentane and dried. 110 mg; ESI-MS [M+H⁺]=191.15.

¹H-NMR (360 MHz, d₆-DMSO) δ ppm: 11.85 (s, 1H), 8.45 (s broad, 3H), 8.40 (s, 1H), 7.60 and 7.30 (each d, 2H), 4.05 (m, 2H), 3.70 (s, 4H).

### N-{[5-Aminomethyl)thiene-3-yl]methyl}-1H-benzimidazole-2-amine (hydrochloride) (21)

Amine 18a (6.5 g; 23.31 mmol) was reacted to the respective aminobenzimidazole by reaction with thiocarbonyldiimidazole, imidazole and then phenylenediamine analogously to the preparation of 17. 1.6 g: ESI-MS [M+H⁺]=359.15. The subsequent cleavage of the Boc-group by means of 4n HCl in dioxane afforded 1.3 g of slightly yellow solids: ESI-MS [M+H⁺]=191.15.

### N¹-Pyridine-2-ylpentane-1,5-diamine (hydrochloride) (22)

Preparation analogously to 18b by reaction of N-1-Boc-1,5-diaminopentane×HCl (5 g; 20.94 mmol) and 20.3 g 2-fluoropyridine, 5.64 g clear oil; ESI-MS [M+H⁺]=280.15. Cleavage of the Boc-group with 4n HCl in dioxane afforded 3.46 g white solids; ESI-MS [M+H⁺]=180.20.

¹H-NMR (360 MHz, d₆-DMSO) δ ppm: 9.10 (s broad, 1H), 8.05 (s broad, 3H), 7.85 (m, 2H), 7.20 (m, 2H), 6.80 (m, 1H), 3.45 (m, superimposed by H₂O), 2.80 (m, 2H), 1.65 (m, 4H), 1.45 (m, 2H).

### N¹-(4,5-Dihydro-1H-imidazole-2-yl)pentane-1,5-diamine (hydrochloride) (23)

N-1-Boc-1,5-diaminopentane×HCl (5 g; 20.94 mmol), 5.4 g ethyldiisopropylamine and 5.11 g 2-(methylsulfanyl)-4,5-dihydro-1H-imidazole×HI in 30 ml DMF were stirred overnight at RT. The reaction mixture was evaporated, taken up in CH₂Cl₂, washed with water and saturated NaCl solution, dried and again evaporated. 5.05 g clear oil, ESI-MS [M+H⁺]: 271.18. Cleavage of the Boc-group with 4n HCl in dioxane and purification of the raw product by means of MPLC afforded 2.57 g; ESI-MS [M+H⁺]: 171.15.

¹³C-NMR (90.55 MHz, d₆-DMSO) δ ppm: 160.3, 43.3 (2 signals superimposed). 42.85, 39.70, 28.90, 27.2, 23.60.

### N-[4-(Aminomethyl)phenyl]-1H-imidazole-2-amine (24)

a) 5.24 g BrCN—dissolved in 50 ml CH₃OH-were added dropwise to a mixture of tert-butyl-4-aminobenzylcarbamate (10 g; 44.99 mmol) and 11.07 g sodium acetate in 100 ml CH₃OH at 0° C., and it was stirred for 3 hours at 0° C. and overnight at RT. The mixture was evaporated, the obtained residue taken up in water and 2× extracted with methyl-tert-butylether. Drying and evaporation of the organic phases afforded 12.99 of a yellow-orange oil.

b) 28.6 g triethylamine were added to 7 g tert-butyl-4-[(iminomethylene)amino]benzyl carbamate in 50 ml pyridine, H₂S was introduced for 1 hour at 0° C. and the mixture was allowed to stand for 48 hours. Evaporation afforded 9.79 g of a rosa foam; ESI-MS [M+H⁺]: 282.05.

¹H-NMR (360 MHz. D₆DMSO) δ ppm: 9.70 (s, 1H), 7.35 (m, 4H), 7.20 (m, 2H), 4.15 (d, 2H), 1.45 (s, 9H).

c) 5 g of the thioamide in 50 ml CH₃OH where methylated with 5.05 g CH₃I and the obtained raw product was directly reacted with 1.73 g aminoacetaldehyde-diethylacetal in 7.5 ml CH₃CN for 3 hours at RT. Evaporation of the reaction mixture afforded 6.36 g of a reddish oil (ESI-MS [M+H⁺]: 381.25) which was dissolved in 50 ml 6n HCl and stirred for 3 hours at 0° C. A pH of 12 was then adjusted with a 25% NaOH solution and it was again stirred for 48 hours at RT. The mixture was extracted 4× with ethyl acetate, the combined organic phases dried and evaporated. The oil thus obtained was stirred 2× with methyl-tert-butylether, the obtained solids filtered with suction and dried. 0.6 g red solid; ESI-MS [M+H⁺]: 189.15.

¹³C-NMR (90.55 MHz, d₆-DMSO) δ ppm: 145.5, 141.60, 130.75, 128.4, 119.8, 115.6, 44.85.

### N¹-(1,4,5,6-Tetrahydropyrimidine-2-yl)pentane-1,5-diamine (hydrochloride) (25)

Preparation was carried out analogously to 23 starting from N-1-Boc-1,5-diaminopentane×HCl (5 g; 20.94 mmol) and 5.4 g 2-(methylsulfanyl)-1,4,5,6-tetrahydropyrimidine×HI. After workup, 1.3 g of a yellowish oil was obtained; [M+H⁺]: 282.2. Cleavage of the Boc-group with 4n HCl in dioxane and purification by means of MPLC afforded 0.46 g; ESI-MS [M+H⁺]: 185.15.

### N-[4-(Aminomethyl)cyclohexyl]pyridine-2-amine (hydrochloride) (26)

Benzyl (4-aminocyclohexyl)methylcarbamate (TFA-salt) (5 g; 13.28 mmol)-preparation by TFA cleavage starting from benzyl-{4-[(tert-butoxycarbonyl)amino]cyclohexyl}methylcarbamate (EP 669317)-was heated to reflux analogously to 18 with 1.71 g ethyldiisopropylamine in 50 ml 2-fluoropyridine. Usual workup and crystallization of the raw products from methyl-tert-butylether/methanol afforded 4.15 g; ESI-MS [M+H⁺]: 340.29.

¹H-NMR (360 MHz, d₆-DMSO) δ ppm: 8.75 (d broad, 1H), 7.85 (m, 2H), 7.35 (m, 5H), 7.05 (d, 1H), 6.85 (m, 1H), 5.05 (s, 2H), 2.90 (m, 2H), 1.95 and 1.75 (each m, 2H), 1.45-0.90 (m, 6H).

Cleavage of the Z group under standard conditions (H₂; Pd-activated carbon), precipitation of the resulting amine as hydrochloride and drying of the obtained precipitates afforded 1.5 g; ESI-MS [M+H⁺]: 206.15.

### tert-Butyl-2,3,4,5-tetrahydro-1H-1-benzazepine-5-ylacetate (27)

75 ml of a 1.0 m BH₃-THF solution were added to a solution of tert-butyl (2-oxo-2,3,4,5-tetrahydro-1H-1-benzazepine-5-yl)acetic acid (2) (10 g; 36.32 mmol) in 100 ml THF and it was stirred at RT. For workup, water was carefully added, it was 2× extracted with diethyl ether and then the organic phases were washed 2× with water. Evaporation and drying afforded 9.3 g; ESI-MS [M+H⁺]: 262.04.

### [5-(2-tert-Butoxy-2-oxoethyl)-2,3,4,5-tetrahydro-1H-1-benzazepine-1-yl]acetic acid (28)

a) 3 g K₂CO₃, 0.05 g KI and 5 g methylbromoacetate were added to a solution of tert-butyl-2,3,4,5-tetrahydro-1H-1-benzazepine-5-yl acetate (27) in 50 ml DMF and it was stirred for 12 hours at 90° C. Thereafter, 4 g methylbromoacetate were added and it was stirred for further 5 hours at 120° C. For workup, the mixture was concentrated, diluted with CH₂Cl₂, washed with saturated NaCl solution, dried and again evaporated. Chromatography on silica gel (CH₂Cl₂/CH₃OH 1-5%) afforded 4.6 g of a bright yellow oil; ESI-MS [M+H⁺]: 334.12.

b) 4.19 of the methylester in 20 ml dioxane/15 ml H₂O were saponified with 19 KOH at RT. For workup, the mixture was concentrated, adjusted to a pH of 2 with 2n HCl and extracted with CH₂Cl₂. The combined organic phases were dried, evaporated and the obtained raw product purified by chromatography on silica gel (CH₂Cl₂/CH₃OH 2-7%).

2.4 g oil, ESI-MS [M+H⁺]: 320.15.

¹H-NMR (360 MHz, CDCl₃) δ ppm: 7.20-7.10 (m, 1H), 6.90 and 6.80 (each m, 1H), 4.0 (s, 2H), 3.55 (m, 1H), 3.10 (m, 2H), 2.85 and 2.70 (each m, 1H), 2.90-2.50 (m, 4H), 1.35 (s, 9H).

### N-[4-(Aminomethyl)cyclohexyl]-1H-imidazole-2-amine (hydrobromide) (29)

Preparation analogously to the preparation of N-[4-(aminomethyl)phenyl]-1H-imidazole-2-amine (23) starting from benzyl-(4-aminocyclohexyl)methylcarbamate (TFA salt) (3 g; 7.97 mmol).

a) Reaction with BrCN and subsequent purification (1.52 g; ESI-MS [M+H⁺]: 288.15).

¹H-NMR (360 MHz, d₆-DMSO) δ ppm: 7.45-7.25 (m, 5H), 6.75 (d, 1H), 5.05 (d, 2H), 2.85 (m, 3H), 1.85 and 1.70 (each m, 2H), 1.35 (m, 1H), 1.20 and 0.95 (each m, 2H).

b) Conversion to the corresponding thiourea and subsequent methylation (1.489; ESI-MS [M+H⁺]: 336.15.

c) Reaction with aminoacetaldehyde-diethylacetal and subsequent cyclization afforded 0.79 g; ESI-MS [M+H⁺]: 329.15.

¹H-NMR (360 MHz, DMSO) δ ppm: 7.45-7.25 (m, 5H), 6.45 (s, 2H), 5.35 (d, 1H), 5.05 (s, 2H), 2.90 (m, 2H), 1.95 and 1.70 (each m, 2H), 1.35 (m, 1H), 1.15 and 0.95 (each m, 2H).

d) For cleavage of the Z-group it was dissolved in 30 ml HBr/glacial acidic acid and stirred for 3 h at RT. For workup, the mixture was evaporated and several times co-evaporated with acetone. 0.89 g; ESI-MS [M+H⁺]: 195.15.

### tert-Butyloxycarbonyl-4-[(2-pyridinylamino)methyl]-1-piperidine (30)

tert-Butyloxycarbonyl-4-(aminomethyl)-1-piperidine (3 g; 14 mmol) and 10 ml 2-fluoropyridine were heated for 4 h to reflux. Evaporation and mixing the raw product in n-pentane afforded 3 g of a wide solid, mp: 126-130° C.; ESI-MS [M+H⁺]=292.15. The amine required for the further reaction was obtained by cleavage of the Boc-group with HCl in dioxane (under standard conditions); the isolated HCl-salt was then directly utilized.

### Ethyl-2-{[5-(2-tert-butoxy-2-oxoethyl)-oxo-2,3,4,6-tetrahydro H-1-benzazepine-1-yl]methyl}-1,3-thiazole-4-carboxylate (31)

a) A solution of t-butyl (2-oxo-2,3,4,5-tetrahydro-1H-1-benzazepine-5-yl)acetic acid (2) in 20 ml DMF was added dropwise to a suspension of 1.28 g NaH (60%; deoiled) in 10 ml DMF at 5° C. and it was stirred for 1 h. 3.49 g Bromoacetonitrile-dissolved in 20 ml DMF-were then added dropwise and it was stirred for 4 h at RT. For workup, water was added carefully, it was diluted with CH₂Cl₂, washed several times with H₂O and saturated NaCl solution, dried and evaporated. Purification of the raw product by chromatography on silica gel (CH₂Cl₂/CH₃OH 5%) afforded 7.61 g; ESI-MS [M+H⁺-^{t}Bu]: 259.05.

b) 5 g tert-Butyl-[1-(cyanomethyl)-2-oxo-2,3,4,5-tetrahydro-1H-1-benzazepine-5-yl]acetate in 70 ml pyridine were saturated for 1 h with H₂S and then allowed to stand overnight at RT. Evaporation of the mixture and mixing of the obtained residue with pentane afforded 5.5 g of a rosa solid which was directly reacted.

c) A mixture of 2 g tert-butyl-[1-(2-amino-2-thioxoethyl)-2-oxo-2,3,4,5-tetrahydro-1H-1-benzazepine-5-yl]acetate, 1.62 g ethylpyruvate and 0.86 g KHCO₃ in 30 ml dioxane was stirred for 2.5 h at RT. Dilution with CH₂Cl₂, washing with saturated NaCl solution, drying and evaporation afforded 2.65 g of a yellow oil; ESI-MS [M+H⁺]: 445.15.

¹H-NMR (360 MHz, d₆-DMSO) δ ppm: 8.50 (s, 1H), 7.50 and 7.35 (each m, 1H), 7.30-7.20 (m, 2H), 7.35 (d, 1H), 5.20 (broad, 1H), 4.30 (q, 2H), 4.20 (m, 1H), 3.65-3.50 (m, 2H), 2.70 (m, 2H), 2.35-2.10 (m, 3H), 1.70-1.50 (m, 2H), 1.30 (s, 9H; superimposed by t, 3H).

### 2-{[5-(2-tert-Butoxy-2-oxoethyl)-2-oxo-2,3,4,5-tetrahydro-1H-1-benzazepine-1-yl]methyl}-1,3-thiazole-4-carbonic acid (32)

2.6 g Ethyl-2-{[5-(2-tert-butoxy-2-oxoethyl)-2-oxo-2,3,4,5-tetrahydro-1H-1-benzazepine-1-yl]methyl}-1,3-thiazole-4-carboxylate (31) was provided in a mixture of 31 ml dioxane and 4 ml H₂O, 1.5 eq. KOH was added and it was heated to reflux. After 5 h, again 1 eq. KOH was added and it was further stirred for 12 h at RT. For workup, it was concentrated, the residue was taken up in water, a pH of 4-5 was adjusted with 2n HCl and it was extracted several times with CH₂Cl₂. The combined org. phases were washed with saturated NaCl solution, dried and evaporated. Mixing with n-pentane afforded 2.1 g of a white solid; ESI-MS [M+H⁺]: 417.15.

¹H-NMR (360 MHz, d₆-DMSO) δ ppm: 8.40 (broad, 1H), 7.5 (m, 1H), 7.35 (m, 1H), 7.30-7.20 (m, 3H), 5.25 (m, 2H), 2.70 (m, 2H), 2.35-2.10 (m, 6H), 1.70 (m, 1H), 1.30 (s, 9H).

### 2-Ammonio-6-(ammoniomethyl)pyridinium trichloride (33)

a) 2-Amino-6-methylpyridine (0.14 mol, 15.0 g) and phthalanhydride (0.14 mol, 20.55 g) were heated to 190° C. at the water separator. Distributing between H₂O and CH₂Cl₂, evaporation of the org. phase and recrystallisation of the residue (diethyl ether) afforded 28.25 g of a slightly yellowish solid; ESI-MS [M+H⁺]=239.15.

¹H-NMR (270 MHz, CDCl₃) δ (ppm): 7.95 (2H, m), 7.78 (3H, m), 7.23 (2H, m), 2.64 (3H, s).

b.) N-bromosuccimide (25.18 mmol, 4.48 g) was added portionwise to a boiling suspension of 2-(6-methyl-2-pyridinyl)-1H-isoindole-1,3(2H)-dione (33a, 20.99 mmol, 5.0 g), AIBN (2.10 mmol, 0.35 g) and dibenzoylperoxide (2.10 mmol, 0.51 g) in CCl₄. The reaction mixture was boiled for 20 h, filtered and the filtrate evaporated. Chromatography (CH₂Cl₂) afforded 3.12 g of the target product and 1.20 g of the dibromo compound; ESI-MS 318.95, 316.95.

¹H-NMR (270 MHz, CDCl₃) δ (ppm): 7.98 (2H, m), 7.95 (1H, t), 7.81 (2H, m), 7.58 (1H, d), 7.35 (1H, d), 4.60 (2H, s).

c) Potassium phthalimide (9.46 mmol, 1.75 g) was added to a solution of 2-[6-(bromomethyl)-2-pyridinyl]-1H-isoindole-1,3(2H)-dione (33b, 6.31 mmol, 2.0 g) in DMF (30 ml), the reaction mixture was heated for 15 h to 60° C., stirred for 24 h at RT, water (60 ml) was added and it was stirred for 2 h at 0° C. The residue was filtrated and washed with a mixture of H₂O-DMF and then with diethyl ether; 2.12 g; ESI-MS [M+H⁺]=384.05.

¹H-NMR (270 MHz, CDCl₃) δ (ppm): 7.98-7.91 (4H, m), 7.88 (1H, t), 7.85-7.75 (4H, m), 7.38-7.32 (2H, m), 5.10 (2H, s).

d) 2-{6-[(1,3-Dioxo-1,3-dihydro-2H-isoindole-2-yl)methyl]-2-pyridinyl}-1H-isoindole-1,3(2H)-dione (33c, 5.22 mmol, 2.09) was heated to reflux for 3 h with hydrazinium hydroxide (13.04 mmol, 0.65 g) in methanol (50 ml). For workup, water was added, it was evaporated and the watery phase acidified with conc. HCl. Anew evaporation and recrystallising (ethyl alcohol) afforded 1.20 g of a white solid; ESI-MS [M+H⁺]=124.05.

¹H-NMR (270 MHz, D₂O) δ (ppm): 7.94 (1H, t), 7.08 (1H, d), 6.99 (1H, d), 4.33 (2H, s).

### trans-N-[4-(Aminomethyl)cyclohexyl]-N′-benzylurea (34)

Preparation was carried out analogously to compound 6 starting from benzyl-{4-[(tert-butoxycarbonyl)amino]cyclohexyl}methylcarbamate (EP 669317) by cleavage of the boc-group with 4n HCl in dioxane. Build-up of the benzyl urea by reaction with benzyl isocyanate and triethylamine in DMF and subsequent hydrogenolysis afforded 0.55 g of the target product; ESI-MS [M+H⁺]=262.20.

### 7-(4-Aminobutyl)-1,2,3,4-tetrahydro[1,8]naphthyridine (bitrifluoroacetate) (35)

a.) A solution of 5-tert-butoxycarbonylaminovaleric acid (50.0 mmol, 10.86 g), O,N-dimethylhydroxylamine hydrochloride (50 mmol, 4.88 g), N-Methylmorpholine (0.30 mol, 30.35 g), HOBT (53.90 mmol, 8.42 g) and EDCI*HCl (55.0 mmol, 10.54 g) in CH₃CN (200 ml) were stirred for 2 days at RT. After evaporation the residue was taken up in ethyl acetate, and then washed with water, a 10% KHSO₄-solution, a saturated watery NaHCO₃ solution and a saturated watery NaCl-solution, subsequently. Drying and evaporation of the organic phase afforded 6.96 g of a yellowish oil; ESI-MS: [2M+Na⁺]=543.3, [M+Na⁺]=283.1, 205.1, 161.1.

¹H-NMR (270 MHz, CDCl₃) δ (ppm): 4.63 (1H, s. br.), 3.68 (3H, s), 3.21-3.05 (3+2H, m), 2.44 (2H, t), 1.76-1.48 (2+2H, m), 1.43 (9H, s).

b.) Methyl magnesia bromide (60.0 mmol, 17.30 ml of a 3M solution in Et₂O) at 0° C. was added dropwise to a solution of tert-butyl 5-[methoxy(methyl)amino]-5-oxopentylcarbamate (35a, 30.0 mmol, 6.9 g) in THF (120 ml) and stirred for 5 h at 0° C. The reaction mixture was then carefully acidified with a 10% KHSO₄-solution, extracted with ethyl acetate and the organic phase then washed with saturated watery NaHCO₃— and saturated watery NaCl-solution, dried and evaporated: 5.5 g yellowish oil; ESI-MS: [M-BOC+H⁺]=116.15.

c.) A mixture of tert-butyl 5-oxohexylcarbamate (35b, 9.29 mmol, 2.0 g), 2-aminonicotinaldehyde (Heterocycl. 1993, 36, 2518; 11.20 mmol, 1.379) and KOH (0.37 ml of a 20% watery solution) was heated to reflux for 8 h. Evaporation and column chromatography afforded 1.60 g of the target product; ESI-MS: [M+H⁺]=302.15.

d.) A suspension of tert-butyl 4-[1,8]naphthyridine-2-ylbutylcarbamate (35c, 5.31 mmol, 1.60 g) and Pd/C (10%, 1.59) in ethyl alcohol (40 ml) were stirred overnight under H₂ atmosphere, then filtered over celite and washed with ethyl alcohol. Column chromatography afforded 290 mg; ESI-MS: [M+H⁺]=306.25.

¹H-NMR (360 MHz, CDCl₃) δ (ppm): 7.04 (1H, d), 6.29 (1H, d), 4.97 (1H, s.br.), 4.81 (1H, s.br.), 3.37 (2H, m sym.), 3.12 (2H, q br.), 2.65 (2H, t), 2.53 (2H, t), 1.89 (2H, quint.), 1.67 (2H, quint.), 1.51 (2H, quint.), 1.43 (9H, s).

e.) TFA (18.30 mmol, 2.099) was added to a solution of tert-butyl 4-(5,6,7,8-tetrahydro[1,8]naphthyridine-2-yl)butylcarbamate (35d, 0.92 mmol, 0.289) in CH₂Cl₂ (8 ml), the solution was stirred for 20 h and evaporated: 380 mg; ESI-MS: 206.1, 130.7.

¹H-NMR (400 MHz, CDCl₃) δ (ppm): 7.07 (1H, d), 6.31 (1H, d), 5.58 (1H, s.br.), 3.39 (2H, m sym.), 2.96 (2H, s br.), 2.76 (2H, t), 2.68 (2H, t), 2.56 (2H, t), 1.88 (2H, quint.), 1.69 (2H, quint.), 1.51 (2H, quint.).

### tert-Butyl [1-(2-hydroxyethyl)-2-oxo-2,3,4,6-tetrahydro-1H-1-benzazepine-5-yl]acetic acid (36)

Tert-butyl (2-oxo-2,3,4,5-tetrahydro-1H-1-benzazepine-5-yl)acetic acid (2) (10.9 mmol, 3.0 g)—dissolved in THF—was added at 0° C. to a solution of diisopropylamine (11.0 mmol, 1.119) and butyl lithium (11.0 mmol, 6.91 ml of a 15% solution in hexane) in THF (100 ml) and the solution further stirred for 1 h. About 100 ml ethylene oxide were then added and the mixture was stirred overnight at RT. The solution was distributed between saturated NH₄Cl and ethyl acetate, the organic phase was washed with water and evaporated; 2.7 g; ESI-MS: [2M+Na⁺]=661.3, [M+K⁺]=358.1, 321.1, [M+H⁺]=320.1, 264.0.

### tert-Butyl [2-oxo-1-(2-oxoethyl)-2,3,4,5-tetrahydro-1H-1-benzazepine-5-yl]acetic acid (37)

tert-Butyl [1-(2-hydroxyethyl)-2-oxo-2,3,4,5-tetrahydro-1H-1-benzazepine-5-yl]acetic acid (36, 6.26 mmol, 2.00 g) dissolved in CH₂Cl₂ was added dropwise within 10 minutes to a solution of oxalyl chloride (7.93 mmol, 1.0 g) and DMSO (16.59 mmol, 1.26 g) in little CH₂Cl₂. After 30 min. triethyl amine (38.22 mmol, 3.87 g) was added, stirred for 5 min., left to reach RT and stirred overnight at RT. For workup, water was added, the mixture extracted with CH₂Cl₂ and the organic phase washed with saturated NaCl—, 1%-H₂SO₄— and with 5%-NaHCO₃-solution. Evaporation afforded 1.8 g of the target product; ESI-MS: 693.2, [M+K⁺]=358.1, 319.1, [M+H⁺]=318.1, 262.0.

### Methyl-2-amino-5-chlorobenzoic acid (38)

Thionyl chloride (0.47 mmol, 55.46 g) was added dropwise at 0° C. to a solution of 2-chloro-5-aminobenzoic acid (0.23 mmol, 40.09) in methanol (400 ml) and the mixture heated to 50-60° C. After the reaction had finished, water was added and it was extracted with ethyl acetate. Then the organic phase was washed with 1n NaOH and diluted HCl solution (pH 1-2), subsequently, and evaporated; 23.0 g; ESI-MS: [M+H⁺]=186.05.

### Methyl 4-chloro-2-[(4-ethoxy-4-oxobutanoyl)amino]benzoic acid (39)

Ethyl succinic acid-chloride (0.14 mol, 22.44 g) in toluen (15 ml) was added dropwise at 0° C. to a solution of methyl 2-amino-5-chloro-benzoic acid (38, 123.9 mmol, 23.0 g) and pyridine (0.26 mol, 20.58 g) in toluen (40 ml). The solution was stirred overnight at RT, water was added and it was extracted with ethyl acetate. The organic phase was washed with 1N HCl-solution, with water, with a saturated NaHCO₃-solution and with a saturated NaCl-solution. Evaporation, recrystallisation (methanol) afforded 34.1 g of the target product; ESI-MS: [2M+Na⁺]=649.0, [M+K⁺]=352.0, [M+H⁺]=314.05; ¹H-NMR (270. MHz, CDCl₃) δ (ppm): 11.06 (1H, s br.), 8.68 (1H, d), 7.99 (1H, m), 7.47 (1H, dd), 4.16 (2H, q), 3.92 (3H, s), 2.74 (4H, m), 1.24 (3H, t).

### Ethyl-7-chloro-5-hydroxy-2-oxo-2,3-dihydro-1H-1-benzazepine-4-carboxylate (40)

Methyl 4-chloro-2-[(4-ethoxy-4-oxobutanoyl)amino]benzoic acid (39, 0.16 mol, 50.20 g) in DMSO (250 ml) was added dropwise at 15° C. to a suspension of deoiled NaH (0.27 mol) in THF (50 ml) and DMSO (80 ml) and the mixture was stirred for 2 h at RT. At 0° C. glacial acetic acid (24 ml) was added and stirred for 20 min. Water (25 ml) was added, the resulting precipitate filtered off, washed with water, taken up in CH₂Cl₂, extracted with water and the organic phase evaporated. The residue was then stirred with diethyl ether (50 ml), filtered and dried; 32 g of a 6:4-mixture methyl/ethyl ester, which was not separated; ESI-MS (Me-ester): [M+K⁺]=307.9, [M+Na⁺]=290.0, [M+H⁺]=268.0; ESI-MS (Et-ester): [M+K⁺]=321.9, [M+Na⁺]=304.0, [M+H⁺]=282.0.

### 7-Chloro-3,4-dihydro-1H-1-benzazepine-2,5-dione (41)

Ethyl-7-chloro-5-hydroxy-2-oxo-2,3-dihydro-1H-1-benzazepine-4-carboxylate (40, 0.11 mol, 32.0 g) was heated to 150° C. in DMSO (500 ml) and water (0.23 mol, 4.09 g) and stirred for 2 h. Water was added at 100° C., the mixture cooled to 0° C. and the resulting precipitate filtered off. Drying afforded 19.09; ESI-MS: [M+Na⁺]=251.1, [M+H⁺]=211.9, 209.95, 130.1.

### tert-Butyl (2E,Z)-(7-Chloro-2-oxo-1,2,3,4-tetrahydro-5H-1-benzazepine-5-ylidene) ethane acid (42)

t-Butyl diethylphosphono acetic acid (0.10 mol, 25.849) was added dropwise at 0° C. to a solution of deoiled NaH (0.10 mol) in DMF (40 ml) and stirred until a clear solution develops. 7-Chloro-3,4-dihydro-1H-1-benzazepine-2,5-dione (41, 90.63 mmol, 19.09) in DMF (185 ml) was added dropwise at 0° C. and stirred at RT. Water was added to the reaction mixture, it was stirred for 1 h and the resulting yellow residue filtered off with suction, washed with water and taken up in CH₂Cl₂. The organic phase was washed with a 5%-NaHCO₃-solution and evaporated. Recrystallisation afforded 23.5 g of the target product; ESI-MS: [2M+H⁺]=615.2, [M+Na⁺]=330.0, 293.0, 254.1, 252.1.

¹H-NMR (400 MHz, CDCl₃) δ (ppm): 9.59 (1H, s br.), 7.45 (1H, m), 7.25 (1H, m), 7.06 (1H, m), 5.99 (1H, t), 3.43 (2H, s), 2.84 (2H, d), 1.32 (9H, s).

### t-Butyl (7-chloro-2-oxo-2,3,4,4-tetrahydro-1H-1-benzazepine-5-yl)acetic acid (43)

tert-Butyl (2E,Z)-(7-chloro-2-oxo-1,2,3,4-tetrahydro-5H-1-benzazepine-5-ylidene)ethane acid (42, 75.0 mmol, 23.089) in ethyl alcohol/dioxane (250 ml/100 ml) was hydrogenated for 4 days with Pt/carbon (5%, 4.1 g) under standard conditions. Water was added to the reaction mixture, stirred for 1 h, the yellow residue filtered off with suction, washed with water and taken up in CH₂Cl₂. The organic phase was washed with a 5%-NaHCO₃-solution and evaporated. Recrystallisation afforded 23.5 g of a solid (mixture of target product and the corresponding dechlorinated compound; the mixture was reacted directly); ESI-MS: [2M+H⁺]=618.94, [M+K⁺]=350.66, 309.75, 254.11.

### Methyl [5-(2-tert-butoxy-2-oxo ethyl)-7-chloro-2-oxo-2,3,4,5-tetrahydro-1H-1-benzazepine-1-yl]acetic acid (44)

tert-Butyl (7-chloro-2-oxo-2,3,4,5-tetrahydro-1H-1-benzazepine-5-yl)acetic acid (43, 60.00 mmol, 18.59 g) in DMF (10 ml) was added dropwise at 15° C. to deoiled NaH (69.00 mmol) in DMF (5 ml) and the mixture was stirred overnight at RT. Ice water was added to the reaction mixture, extracted (2×) with ethyl acetate and the organic phase washed with a 10% CH₃COOH-solution, with water and then with 1n NaOH. Evaporation afforded 20.4 g of a raw product, which was reacted without further purification.

### [5-(2-tert-Butoxy-2-oxoethyl)-7-chloro-2-oxo-2,3,4,5-tetrahydro-1H-1-benzazepine-1-yl]acetic acid (45)

KOH (80.45 mmol, 4.51 g) in water (150 ml) was added to methyl [5-(2-tert-butoxy-2-oxoethyl)-7-chloro-2-oxo-2,3,4,5-tetrahydro-1H-1-benzazepine-1-yl]acetic acid (44, 50.28 mmol, 19.29) dissolved in dioxane (250 ml) and stirred for 1 h at RT. The reaction mixture was evaporated, water (100 ml) was added and it was extracted with ethyl acetate (2×). After the evaporation, the residue was taken up in diethyl ether and precipitated by addition of n-pentane. Recrystallisation (diisopropyl ether, 2×) afforded 4.8 g (comprises about 15% of the corresponding dechloro compound); ESI-MS: [M+Na⁺]=390.0, 314.0, 312.0.

### [4-(Aminomethyl)phenyl]guanidine (Bihydrochloride) (46)

p-Aminobenzylamine (6.7 g; 54.84 mmol) was suspended in 20 ml 6n HCl and 5.3 g cyanamide—dissolved in 5 ml H₂0—added slowly under reflux. After the reaction had completed, 50% NaOH-solution was added at 0° C. to the solution, the resulting precipitate filtered with suction, boiled up in 50 ml ethyl alcohol and filtered off. Evaporation of the mother liquor and mixing of the obtained residue with diethyl ether afforded 1.4 g of yellow solids; Fp.: 255° C.

### 7,8-Dimethoxy-3,4-dihydro-1H-1-benzazepine-2,5-dione (47)

Analogously to the preparation of the corresponding building blocks 39, 40 and 41 first ethyl-2-amino-4,5-dimethoxybenzoate (20 g; 88.8 mmol) was reacted with ethyl succinic acid-chloride to the respective amide. After mixing with n-pentane 30.4 g of a solid was obtained; ESI-MS [M+H⁺]: 354.15.

¹H-NMR (400 MHz, DMSO) δ (ppm): 10.65 (s, 1H), 8.10 and 7.40 (each s, 1H), 4.30 and 4.10 (each q, 2H), 3.85 and 3.80 (each s, 3H), 2.70 (m, 4H), 1.30 and 1.20 (each t, 3H). Subsequent cyclization with 25 g of the obtained amide under utilization of NaH analogously to 40 and usual workup afforded 19.5 g of a white solid; ESI-MS [M+H⁺]: 308.05.

¹H-NMR (400 MHz, DMSO) δ (ppm): 13.3 (s, 1H), 10.10 (s, 1H), 7.25 and 6.75 (each s, 1H), 4.30 (q, 2H), 3.80 (s, 6H), 2.95 (s, 2H), 1.35 (t, 3H).

Decarboxylation analogously to 41 starting from 17 g afforded 10.5 g of the target product as a solid; ESI-MS [M+H⁺]: 236.15.

¹H-NMR (400 MHz, DMSO) δ (ppm): 9.90 (s, 1H), 7.35 and 6.80 (each s, 1H), 3.80 and 3.75 (each s, 3H), 2.85 and 2.65 (each m, 2H).

### tert-Butyl (7,8-dimethoxy-2-oxo-2,3,4,5-tetrahydro-1H-1-benzazepine-5-yl)acetate (48)

Preparation analogously to building blocks 42 and 43 by Wittig-Homer-reaction and subsequent hydrogenation; after mixing with n-pentane 8.16 g of solids were obtained; ESI-MS [M+H⁺-^{t}Bu]: 280.15.

### [5-(2-tert-Butoxy-2-oxoethyl)-7,8-dimethoxy-2-oxo-2,3,4,5-tetrahydro-1H-1-benzazepine-1-yl]acetate (49)

Analogously to 44 and 45, starting from 4 g tert-butyl-(7,8-dimethoxy-2-oxo-2,3,4,5-tetrahydro-1H-1-benzazepine-5-yl)acetate 2.6 g of the target product were isolated as a bright foam; [M+H⁺-^{t}Bu]: 338.15.

¹H-NMR (400 MHz, DMSO) δ (ppm): 6.85 and 6.75 (each s, 1H), 4.35 (s broad, 2H), 3.80 and 3.75 (each s, 3H), 3.60 (s, 2H), 2.70 (m, 2H), 2.25 (m, 1H), 2.15 (m, 2H), 1.60 (m, 1H), 1.35 (s, 9H).

### [1-(2-Methoxy-2-oxoethyl)-2-oxo-2,3-dihydro-1H-1-benzazepine-5-yl]acetic acid (50)

a.) 56.1 g (406 mmol) powdered K₂CO₃ were added at room temperature to a solution of 37 g (135.3 mmol) t-butyl (2-oxo-2,3-dihydro-1H-1-benzazepine-5-yl)acetic acid (1) and 3.7 g tetrabutylammoniumbromide in 370 ml DMF, 22.7 g (148.9 mmol) methylbromoacetic acid was added dropwise, then it was stirred for 3 h at 40° C. and overnight at room temperature. The reaction mixture was poured into 1000 ml of a water-ice-mixture, and 2× extracted with each 200 ml methyl-tert.butyl ether. The combined extracts were washed with H₂O, 5% NaHCO₃— and NaCl-solution, dried over Na₂SO₄, filtered off and evaporated. The remaining yellow oil (about 52 g, purity about 90%) was reacted without further purification; ESI-MS [M+H⁺]: 346.

b.) 9.2 g (26.6 mmol) of the raw product 50a were dissolved in 66.6 ml 4n HCl in dioxane and stirred for 24 h at 50° C., then the dioxane was extensively distilled off, 5% NaHCO₃-solution and diethyl ether added to the residue, the watery phase again washed with diethyl ether and acidified with 1n KHSO₄-solution. The precipitating acid was extracted with diethyl ether, the ether phase washed with a NaCl-solution, dried over Na₂SO₄, filtered off with suction and evaporated. It remained 3.2 g of a slightly yellow oil; ESI-MS [M+H⁺]: 290.

### trans-[4-(1H-Benzimidazole-2-yl)cyclohexyl]methaneamine (hydrochloride) (51)

39.3 g (0.25 mol) trans-4-(Aminomethyl)cyclohexanecarbonic acid and 27.0 g (0.25 mol) 1,2-phenylene diamine were heated to reflux for 18 h in a mixture of 167 ml conc. hydrochloric acid and 333 ml H₂O analogously to J. Heterocycl. Chem. 26, 541 (1989). The green reaction solution was concentrated until the occurrence of a yellow crystalline pulp and stirred with 400 ml isopropanol, filtered off, washed with 90% isopropanol and finally with diethyl ether. After 2 times recrystallisation from a isopropanol-water mixture (70/30), 30 g of a white monohydrochloride remained; ESI-MS [M+H⁺]: 230.

### Methyl (2-oxo-2,3,4,5-tetrahydro-1H-1,5benzodiazepine-1-yl)acetic acid (52)

1.99 (79.8 mmol) NaH (60% dispersion in mineral oil) were added to an ice cooled solution of 12.2 g (75.3 mmol) 1,3,4,5-tetrahydro-2H-1,5-benzodiazepine-2-One (preparation: J. Am. Chem. Soc. 1949, 71, 1985) in 350 ml DMF, stirred for 30 min. at 0-5° C. and for 10 min. at room temperature. Then 11.5 g (75.3 mmol) methyl bromo acetic acid were added dropwise at 0° C. and it was then stirred for 30 min. at the same temperature. The reaction solution was poured onto 600 ml ice water and 3× extracted with each 150 ml ethyl acetate. The organic phase was washed with a NaCl-solution, dried over MgSO₄ and ethyl acetate was distilled off. The residue was purified by column chromatography (eluent: CH₂Cl₂/CH₃OH 9/1). 9.6 g of a yellowish oil were isolated; ESI-MS [M+H⁺]: 235.

### tert-butyl (2-oxo-2,3,4,6-tetrahydro-1H-1,5-benzodiazepine-1-yl)acetic acid (53)

Preparation was carried out analogously to building block 52 by reaction of 11.99 (73 mmol) 1,3,4,5-tetrahydro-2H-1,5-benzodiazepine-2-one with 14.3 g (73 mmol) tert-butyl bromo acetic acid. 17 g of a slightly yellowish oil were isolated; ESI-MS [M+H⁺]: 277.

### [5-(2-tert-Butoxy-2-oxoethyl)-2-oxo-2,3,4,6-tetrahydro-1H-1,5-benzodiazepine-1-yl]acetic acid (54)

a.) 14.2 g (102 mmol) powdered K₂CO₃ were added at 0° C. to a solution of 9.6 g (41 mmol) compound 52 and 8.09 (41 mmol) tert-butyl bromo acetic acid in 90 ml DMF, and stirred for 1 h at 0° C. and then for 14 h at room temperature. The reaction mixture was poured onto 300 ml ice water and 3× extracted with each 100 ml methyl-tert.butyl ether. The combined organic phases were washed several times with a NaCl-solution, dried over MgSO₄ and evaporated to dryness. The residue was purified by column chromatography (eluent: ethyl acetate/cyclohexane 7/3). 7.0 g of a slightly yellowish oil were isolated; ESI-MS [M+H⁺]: 349.

b.) The alkaline saponification of the methyl ester was carried out analogously to 3b. 3.8 g white crystals were obtained; Fp.: 140-142° C.; ESI-MS [M+H⁺]: 335.

### [5-(2-tert-Butoxy-2 oxoethyl)-4-oxo-2,3,4,6-tetrahydro-1H-1,5-benzodiazepine-1-yl]acetic acid (55)

Analogously to building block 54a tert-butyl (2-oxo-2,3,4,5-tetrahydro-1H-1,5-benzodiazepine-1-yl)acetic acid (53) was reacted with methyl bromoacetic acid and then alkaline saponified analogously to 3b. After purification by column chromatography 2.9 g white crystals were obtained; Fp.: 82-84° C.; ESI-MS [M+H⁺]: 335.

### [1-(2-Methoxy-2-oxoethyl)-2-oxo-2,3,4,5-tetrahydro-1H-1-benzazepine-5-yl]acetic acid (56)

14.5 g (42 mmol) building block 3a were dissolved in 105 ml 4n HCl in dioxane and stirred for 2 days at 50° C. After evaporation of the solvent, the residue was dissolved in 5% NaHCO₃-solution, 2× extracted with methyl-tert.butyl ether, then the watery phase was acidified with a 1n KHSO₄-solution and the precipitating acid extracted with methyl-tert.butyl ether. After drying over MgSO₄, evaporation of the solvent and purification by column chromatography (eluent: CH₂Cl₂/CH₃OH/glacial acetic acid 451511) 1.6 g of a viscous, slightly yellowish oil remained; ESI-MS [M+H⁺]: 292.

### [(5R)-5-(2-tert-Butoxy-2-oxoethyl)-2-oxo-2,3,4,5-tetrahydro-1H-1-benzazepine-1-yl]acetic acid (57)

14.2 g (42.6 mmol) [5-(2-t.Butoxy-2-oxoethyl)-2-oxo-2,3,4,5-tetrahydro-1H-benzazepine-1-yl]acetic acid (3) were suspended in 170 ml diethylether and dissolved by addition of 7.3 g (42.64 mmol) (1S)-(−)-1-napthyl)ethylamine. The yellow solution was inoculated with (1S)-1-napthyl)ethaneaminium[(5R)-5-(2-tert.butoxy-2-oxoethyl)-2-oxo-2,3,4,5-tetrahydro-1H-benzazepine-1-yl]acetate—prepared by preparative HPLC-separation of compound 3 by means of a chiral column (Chiralpak AD 500×; 50 mm; 20 μm) and subsequent salification-, the deposited precipitate filtered off with suction after 3.5 h and 3× recrystallized from a ethyl acetate/isopropanol mixture. The purity of enantiomers was checked by means of a chiral HPLC. 3.5 g. of the salt were suspended in 30 ml of a diethylether/hexane-mixture 10/3 and after addition of 50 ml of a 5% watery amidosulfonic acid solution stirred until occurrence of a clear phase. After separation of the watery phase, the organic phase was washed 3× with 5 ml amidosulfonic acid- and then with a NaCl-solution, dried over Na₂SO₄ and evaporated. 2.25 g amorphous residue; ESI-MS [M+H⁺]: 505.

### [(5S)-5-(2-tert-Butoxy-2-oxoethyl)-2-oxo-2,3,4,5-tetrahydro-1H-1-benzazepine-1-yl]acetic acid (58)

From the combined mother liquors of building block 57, 8 g of the acid were isolated as a yellowish amorphous residue as described with a amidosulfonic acid solution, said acid was reacted with (1R)-(+)-1-napthyl)ethyl amine into the diastereomeric salt and recrystallized until the achievement of enantiomeric purity. Analogously to example 57, 2.5 g of the dextrorotatory acid were isolated as an amorphous solid, ESI-MS [M+H⁺]: 505.

A sample of the acid was reacted with 4-bromobenzylamine into the good crystallizing amide and the absolute configuration figured out by means of a x-ray structure analysis.

### tert-Butyl-(5-oxo-5,6-dihydro-4H-thieno[3,2-b]azepine-8-yl)acetate (59)

A solution of 5.3 g (29.2 mmol) 6,7-dihydro-4H-thieno[3,2-b]azepine-5,8-dione (Arch. Pharm. 1991 324, 579) and 12 g (32.2 mmol) (tert-butoxycarbonylmethylene)-triphenyl phosphorane in 15 ml toluen was heated to reflux for 10 h, then toluen was distilled off and the black residue purified by chromatography (eluent: ethyl acetate/cyclohexane 7/3). The consistent fraction was again digested with 40 ml boiling cyclohexane, cooled and filtered with suction. 3 g yellowish crystals; ESI-MS [M+H⁺]: 280.

### tert-Butyl-(5-oxo-5,6,7,8-tetrahydro-4H-thieno[3,2-b]azepine-8-yl)acetate (60)

39 (11 mol) tert-Butyl-(5-oxo-5,6-dihydro-4H-thieno[3,2-b]azepine-8-yl)acetate were hydrogenated analogously to building block 2 in the presence of 10% Pd/C. Since educt was still present after 6 h according to HPLC, the catalyst was filtered with suction and after addition of new catalyst it was again hydrogenated for 6 h. After workup and chromatographic purification (eluent: ethyl acetate/cyclohexane 7/3), 1.4 g yellowish crystals were isolated, which comprised according to HPLC still about 2.5% educt; ESI-MS [M+H⁺]: 282.

### [8-(2-tert-Butoxy-2-oxoethyl)-5-oxo-5,6,7,8-tetrahydro-4H-thieno[3,2-b]azepine-4-yl]acetic acid (61)

Preparation was carried out analogously to building block 50.

Ester stage: 1.5 g yellowish crystals; ESI-MS [M+H⁺]: 354.

Target product: 1.2 g yellowish crystals; ESI-MS [M+H⁺]: 340.

### I.B Compounds of the Formula I or I′

### Example I

### t-Butyl [1-(2-{[(2-{[amino(imino)methyl]amino}-1,3-thiazol-5-yl)methyl]amino}-2-oxoethyl)-2-oxo-2,3,4,5-tetrahydro-1H-1-benzazepin-5-yl]acetate

1.1 g of N-methylmorpholine were added dropwise at 0° C. to 1.5 g (4.65 mmol) of [5-(2-t-butoxy-2-oxoethyl)-2-oxo-2,3,4,5-tetrahydro-1H-benzazepin-1-yl]acetic acid (3) and 1.22 g (4.8 mmol) of N-[5-(aminomethyl)-1,3-thiazol-2-yl]guanidine dihydrochloride (5) in 20 ml of DMF, and 1.55 g (4.7 mmol) of TOTU (O-[(ethoxycarbonyl)cyanomethyleneamino]-N,N,N′,N′-tetrafluoroborate) were then introduced in portions over the course of 35′. The yellow reaction solution was stirred at 0° C. for 1 h and then largely evaporated in vacuo. The residue was then digested a number of times with H₂O, taken up in a mixture of 120 ml of ethyl acetate and 40 ml of diethyl ether, washed with 10% K₂CO₃ and NaCl solution, dried over Na₂SO₄ and concentrated, the crude product thoroughly crystallizing. Purification by chromatography on silica gel (CH₂Cl₂/CH₃OH/NH₃ 42:8:0.1) and crystallization from ethyl acetate/n-hexane afforded 1.45 g (65%) of white crystals.

M.p.: 190-193° C. (dec.); FAB-MS [M+H⁺]: 487.

### Example II

### [1-(2-{[(2-{[Amino(imino)methyl]amino}-1,3-thiazol-5-yl)methyl]amino}-2-oxoethyl)-2-oxo-2,3,4,5-tetrahydro-1H-1-benzazepin-5-yl]acetic acid

1.2 g of the t-butyl ester from Example I were suspended in 70 ml of CH₂Cl₂, treated with 45 ml of 4N HCl in dioxane and stirred overnight at RT. The solution was evaporated, and the residue was digested a number of times with CH₂Cl₂ and then dried. In this way, 1.07 g of a slightly yellowish amorphous powder were isolated; FAB-MS [M−H⁺]: 432.

### Example III

### [1-(2-{[4-(1H-Benzimidazol-2-ylamino)benzyl]amino}-2-oxoethyl)-2-oxo-2,3,4,5-tetrahydro-1H-1-benzazepin-5-yl]acetic acid

Preparation was carried out analogously to Example I by reaction of [5-(2-t-butoxy-2-oxoethyl)-2-oxo-2,3,4,5-tetrahydro-1H-benzazepin-1-yl]acetic acid (3) with N-[4-(aminomethyl)phenyl]-1H-benzimidazole-2-amine hydrochloride (4) and subsequent removal of the t-butyl group analogously to Example II. A slightly yellowish amorphous residue was obtained, FAB-MS [M−H⁺]: 554.

### Example IV

### t-Butyl [1-(2-{[(2-{[amino(imino)methyl]amino}-1,3-thiazol-5-yl)methyl]amino}-2-oxoethyl)-2-oxo-2,3-dihydro-1H-1-benzazepin-5-yl]acetate

Analogously to the preparation of compound 3a, 0.9 g (3.3 mmol) of t-butyl (2-oxo-2,3-dihydro-1H-1-benzazepin-5-yl)acetate (1) was alkylated with methyl bromoacetate (FAB-MS [M−H⁺]: 346) and then hydrolyzed analogously to 3b (0.44 g; FAB-MS [M−H⁺]: 332). Coupling of 0.57 g (1.72 mmol) of [5-(2-t-butoxy-2-oxoethyl)-2-oxo-2,3-dihydro-1H-1-benzazepin-1-yl]acetic acid with N-[5-(aminomethyl)-1,3-thiazol-2-yl]guanidine dihydrochloride (5) analogously to Example I afforded the title compound as a slightly yellowish powder; FAB-MS [M−H⁺]: 485.

### Example V

### [1-(2-{[(2-{[Amino(imino)methyl]amino}-1,3-thiazol-5-yl)methyl]amino}-2-oxoethyl)-2-oxo-2,3-dihydro-1H-1-benzazepin-5-yl]acetic acid

The t-butyl ester was removed analogously to Example II and afforded 0.42 g of the title compound as a slightly yellowish powder; FAB-MS [M−H⁺]: 429.

### Example VI

### (1-{2-[(4-{[(Benzylamino)carbonyl]amino}benzyl)amino]-2-oxoethyl}-2-oxo-2,3,4,5-tetrahydro-1H-1-benzazepin-5-yl)acetic acid

Preparation was carried out analogously to Example I by reaction of [5-(2-t-butoxy-2-oxoethyl)-2-oxo-2,3,4,5-tetrahydro-1H-benzazepin-5-yl]acetic acid (3) with N-[4-(aminomethyl)phenyl]-N′-benzylurea (6) and subsequent removal of the t-butyl group analogously to Example II. Purification of the crude product by elution through a silica gel cartridge (Chromasorb; CH₂Cl₂/CH₃OH 0-20%) afforded 27 mg as an amorphous solid; ESI-MS [M+H⁺]: 515.2; [M+K⁺]: 553.2.

### Example VII

### [1-(2-{[4-(1H-Benzimidazol-2-yl)benzyl]amino}-2-oxoethyl)-2-oxo-2,3,4,5-tetrahydro-1H-1-benzazepin-5-yl]acetic acid

Preparation was carried out analogously to Example I by reaction of [5-(2-t-butoxy-2-oxoethyl)-2-oxo-2,3,4,5-tetrahydro-1H-benzazepin-1-yl]acetic acid (3) with [4-(1H-benzimidazol-2-yl)phenyl]methaneamine (7) and subsequent cleavage of the t-butyl group analogously to Example II. Purification of the crude product by elution through a silica gel cartridge (Chromasorb; CH₂Cl₂/CH₃OH 0-20%) afforded 9 mg as an amorphous solid; ESI-MS [M+H⁺]: 485.2.

### Example VIII

### [1-(2-{[4-(1H-Benzimidazol-2-ylamino)butyl]amino}-2-oxoethyl)-2-oxo-2,3,4,5-tetrahydro-1H-1-benzazepin-5-yl]acetic acid

Preparation was carried out analogously to Example I by reaction of [5-(2-t-butoxy-2-oxoethyl)-2-oxo-2,3,4,5-tetrahydro-1H-benzazepin-5-yl]acetic acid (3) with N¹-(1H-benzimidazol-2-yl)butane-1,4-diamine (trifluoroacetate) (9) and subsequent cleavage of the t-butyl group analogously to Example II. After chromatographic purification on silica gel (eluent: CH₂Cl₂/CH₃OH/50% acetic acid 42:8:0.7), 0.5 g was isolated as a slightly yellowish amorphous powder; FAB-MS [M−H⁺]: 464.

### Example IX

### [1-(2-{[5-(1H-Benzimidazol-2-ylamino)pentyl]amino}-2-oxoethyl)-2-oxo-2,3,4,5-tetrahydro-1H-1-benzazepin-5-yl]acetic acid

Preparation was carried out analogously to Example I by reaction of [5-(2-t-butoxy-2-oxoethyl)-2-oxo-2,3,4,5-tetrahydro-1H-benzazepin-1-yl]acetic acid (3) with N¹-(1H-benzimidazol-2-yl)pentane-1,5-diamine (hydrochloride) (8) and subsequent cleavage of the t-butyl group analogously to Example II. After chromatographic purification on silica gel (eluent: CH₂Cl₂/CH₃OH/50% acetic acid 42:8:0.7), 0.48 g was isolated as a slightly yellowish amorphous powder; FAB-MS [M−H⁺]: 478.

### Example X

### {1-[2-({4-[(1H-Benzimidazol-2-ylamino)methyl]cyclohexyl}amino)-2-oxoethyl]-2-oxo-2,3,4,5-tetrahydro-1H-1-benzazepin-5-yl}acetic acid

Preparation was carried out analogously to Example I by reaction of [5-(2-t-butoxy-2-oxoethyl)-2-oxo-2,3,4,5-tetrahydro-1H-benzazepin-1-yl]acetic acid (3) with trans-N-[(4-aminocyclohexyl)methyl]-1H-benzimidazole-2-amine (dihydrochloride) (10) and subsequent cleavage of the t-butyl group analogously to Example II. After chromatographic purification on silica gel, 0.7 g of slightly yellowish amorphous powder was isolated; FAB-MS [M+H⁺]: 504.

### Example XI

### {1-[2-({[4-(1H-Benzimidazol-2-ylamino)cyclohexyl]methyl}amino)-2-oxoethyl]-2-oxo-2,3,4,5-tetrahydro-1H-1-benzazepin-5-yl}acetic acid

Preparation was carried out analogously to Example I by reaction of [5-(2-t-butoxy-2-oxoethyl)-2-oxo-2,3,4,5-tetrahydro-1H-benzazepin-5-yl]acetic acid (3) with trans-N-{[4-(aminomethyl)cyclohexyl]-1H-benzimidazole-2-amine (dihydrochloride) (11) and subsequent cleavage of the t-butyl group analogously to Example II. After chromatographic purification on silica gel, 0.5 g of slightly yellowish amorphous powder was isolated; FAB-MS [M+H⁺]: 504.

### Example XII

### [2-Oxo-1-(2-oxo-2-{[4-(pyridin-2-ylamino)benzyl]amino}ethyl)-2,3,4,5-tetrahydro-1H-1-benzazepin-5-yl]acetic acid

Preparation was carried out analogously to Example I by reaction of [5-(2-t-butoxy-2-oxoethyl)-2-oxo-2,3,4,5-tetrahydro-1H-benzazepin-1-yl]acetic acid (3) with N-[4-(aminomethyl)phenyl]-2-pyridineamine (13) and subsequent cleavage of the t-butyl group analogously to Example II (14 mg); ESI-MS [M+H⁺]: 459.15.

### Example XIII

### {1-[2-({[6-(1H-Benzimidazol-2-yl)pyridin-3-yl]methyl}amino)-2-oxoethyl]-2-oxo-2,3,4,5-tetrahydro-1H-1-benzazepin-5-yl}acetic acid (bishydrochloride)

Preparation was carried out analogously to Example I by reaction of [5-(2-t-butoxy-2-oxoethyl)-2-oxo-2,3,4,5-tetrahydro-1H-benzazepin-1-yl]acetic acid (3) with [6-(1H-benzimidazol-2-yl)pyridin-3-yl]methaneamine (trifluoroacetate) (12) and subsequent cleavage of the t-butyl group analogously to Example II. (13 mg); ESI-MS [M+H⁺]: 484.15.

Compounds of the general formula I analogously to example II were prepared:

### Example 14

### {2-Oxo-1-[2-oxo-2-({4-[(pyridine-2-ylamino)methyl]benzyl}amino)ethyl]-2,3,4,5-tetrahydro-1H-1-benzazepine-5-yl}acetic acid

Under utilization of N-[4-(aminomethyl)benzyl]-2-pyridine amine (14) as educt, 207 mg as an amorphous solid were obtained according to MPLC 207 mg; ESI-MS [M+H⁺]: 473.28.

¹H-NMR (360 MHz, d₆-DMSO) δ ppm: 8.5 (m, 1H), 7.95 (d, 1H), 7.45 (m, 1H), 7.35-72.0 (m, 9H), 6.60 (m, 2H), 4.45 (m, 3H), 4.25 (m, 3H), 3.55 (m, 1H), 2.70 (m, 2H), 2.35 (m, 1H), 2.20 (m, 2H), 1.65 (m 1H).

### Example 15

### {1-[2-({[5-(1H-Benzimidazole-2-yl)thiene-2-yl]methyl}amino)-2-oxoethyl]-2-oxo-2,3,4,5-tetrahydro-1H-1-benzazepine-5-yl}acetic acid

Under utilization of [5-(1H-benzimidazole-2-yl)thiene-2-yl]methaneamine (15) as educt, 210 mg as amorphous white solids were obtained according to MPLC; ESI-MS [M+H⁺]: 489.27.

¹H-NMR (360 MHz, d₆-DMSO) δ ppm: 8.80 (m, 1H), 7.70 (m, 1H), 7.65 (m, 2H), 7.35-7.15 (m, 6H), 7.05 (m, 1H), 4.55 (m, 3H), 4.20 (m, 1H), 3.55 (m, 1H), 2.75 (m, 2H), 2.35 (m, 1H), 2.15 (m, 2H), 1.65 (m, 1H).

### Example 16

### {1-[2-({2-[4-(1H-Benzimidazole-2-yl)phenyl]ethyl}amino)-2-oxoethyl]-2-oxo-2,3,4,6-tetrahydro-1H-1-benzazepine-5-yl}acetic acid

Under utilization of tert-butyl-2-[4-(1H-benzimidazole-2-yl)phenyl]ethylcarbamate (16) as educt, 190 mg as amorphous white solids were obtained according to MPLC; ESI-MS [M+H⁺]: 497.15.

¹H-NMR (360 MHz, d₆-DMSO) δ ppm: 8.15 (d, 2H), 7.65 (d, 2H), 7.45 (m, 2H), 7.30-7.15 (m, 6H), 4.45 and 4.15 (each m, 1H), 3.6-3.25 (m, superimposed by H₂O), 2.80 (m, 2H), 2.70 (m, 2H), 2.35 (m, 1H), 2.20 (m, 2H), 1.70 (m, 1H).

### Example 17

### [1-(2-{4-[(1H-Benzimidazole-2-ylamino)methyl]piperidine-1-yl}-2-oxoethyl)-2-oxo-2,3,4,5-tetrahydro-1H-1-benzazepine-5-yl]acetic acid

Under utilization of (3 N-(piperidine-4-ylmethyl)-1H-benzimidazole-2-amine (trifluoro acetate) (17) as educt, 134 mg as amorphous white solids were obtained according to MPLC; ESI-MS [M+H⁺]: 490.29.

### Example 18

### [2-Oxo-1-(2-oxo-2-{[2-(pyridine-2-ylamino)ethyl]amino}ethyl)-2,3,4,6-tetrahydro-1H-1-benzazepine-5-yl]acetic acid

Under utilization of N¹-pyridine-2-ylethane-1,2-diamine, the subsequent MPLC afforded 278 mg as amorphous white solids; ESI-MS [M+H⁺]: 397.25.

¹H-NMR (360 MHz, d₆-DMSO) δ ppm: 8.20 (m, 1H), 7.95 (m, 1H), 7.70 (m, 2H), 7.35-7.20 (m, 4H) 6.80 and 6.70 (each m, 1H), 4.45 and 4.15 (each m, 1H), 3.70-3.0 (m, superimposed by H₂O), 2.70 (m, 2H), 2.30 (m, 1H), 2.15 (m, 2H), 1.60 (m, 1H).

### Example 19

### (2-Oxo-1-{2-oxo-2-[({4-[(pyridine-2-ylamino)methyl]thiene-2-yl}methyl)amino]ethyl}-2,3,4,5-tetrahydro-1H-1-benzazepine-5-yl)acetic acid

Under utilization of N-{[5-(aminomethyl)thiene-3-yl]methyl}pyridine-2-amine (trifluoro acetate) (18), the subsequent MPLC afforded 135 mg as amorphous white solids; ESI-MS [M+H⁺]: 479.15.

¹H-NMR (360 MHz, d₆-DMSO) δ ppm: 8.65 (m, 1H), 8.0 (m, 1H), 7.45 (m, 1H), 7.35-7.15 (m, 6H), 6.95 (s, 1H), 6.60 and 6.55 (each m, 1H), 4.60-4.30 (m, 5H), 4.15 (m 1H), 3.50 (m, 1H), 2.80-2.60 (m, 2H), 2.35 (m, 1H), 2.15 (m, 2H), 1.70 (m, 1H).

### Example 20

### [1-(2-{[3-(1H-imidazole-2-ylamino)-3-oxopropyl]amino}-2-oxoethyl)-2-oxo-2,3,4,5-tetrahydro-1H-1-benzazepine-5-yl]acetic acid

Under utilization of 3-amino-N-(1H-imidazole-2-yl)propane amide (19) and following purification of the raw product by MPLC 50 mg were obtained as amorphous white solids; ESI-MS [M+H⁺]: 414.25.

¹H-NMR (360 MHz, d₆-DMSO) δ ppm: 8.20 (m, 1H), 7.30-7.20 (m, 7H), 4.45 and 4.15 (each m, 1H), 3.75-3.25 (m, superimposed by H₂O), 2.80-2.65 (m, 4H), 2.35 (m, 1H), 2.15 (m, 2H), 1.60 (m, 1H).

### Example 21

### [1-(2-{[4-(4,5-Dihydro-1H-imidazole-2-ylamino)benzyl]amino}-2-oxoethyl)-2-oxo-2,3,4,5-tetrahydro-1H-1-benzazepine-5-yl]acetic acid

Under utilization of N-[4-(aminomethyl)-phenyl]-4,5-dihydro-1H-imidazole-2-amine (hydrochloride) (20) as educt, 12 mg were obtained as amorphous white solids after MPLC; ESI-MS [M+H⁺]: 450.3.

### Example 22

### (1-{2-[({4-[(1H-Benzimidazole-2-ylamino)methyl]thiene-2-yl}methyl)amino]-2-oxoethyl}-2-oxo-2,3,4,5-tetrahydro-1H-1-benzazepine-6-yl)acetic acid

Utilization of N-{[5-(aminomethyl)thiene-3-yl]methyl}-1H-benzimidazole-2-amine (hydrochloride) (21) as educt; purification by MPLC afforded 90 mg; ESI-MS [M+H⁺]: 518.29.

### Example 23

### [2-Oxo-1-(2-oxo-2-{[5-(pyridine-2-ylamino)pentyl]amino}ethyl)-2,3,4,5-tetrahydro-1H-1-benzazepine-5-yl]acetic acid

Utilization of N¹-pyridine-2-ylpentane-1,5-diamine (hydrochloride) (22) as educt; after MPLC, 210 mg white solids were obtained; ESI-MS [M+H⁺]: 439.15.

¹H-NMR (360 MHz, d₆-DMSO) δ ppm: 8.05 (m, 1H), 7.95 (m, 1H), 7.75 (m broad, 1H), 7.65 (m, 1H), 7.30-7.15 (m, 4H), 6.75 and 6.60 (each m, 1H), 4.45 and 4.15 (each m, 1H), 3.70-3.20 (m, superimposed by H₂O), 2.70 (m, 2H), 2.35 (m, 1H), 2.15 (m, 2H), 1.60, 1.45 and 1.30 (each m, 2H).

### Example 24

### N-[5-({[5-(Carboxymethyl)-2-oxo-2,3,4,5-tetrahydro-1H-1-benzazepine-1-yl]acetyl}amino)pentyl]-4,5-dihydro-1H-imidazole-2-amine (acetate)

Utilization of N¹-(4,5-Dihydro-1H-imidazole-2-yl)pentane-1,5-diamine (hydrochloride) (23) as educt; 22 mg were obtained after MPLC; ESI-MS [M+H⁺]: 430.15.

### Example 25

### [1-(2-{[4-(1H-imidazole-2-ylamino)benzyl]amino}-2-oxoethyl)-2-oxo-2,3,4,5-tetrahydro-1H-1-benzazepine-5-yl]acetic acid

Utilization of N-[4-(aminomethyl)phenyl]-1H-imidazole-2-amine (24) as educt; purification by means of MPLC afforded 40 mg; ESI-MS [M+H⁺]: 448.15.

¹H-NMR (360 MHz, d₆-DMSO) δ ppm: 8.95 (s, 1H), 8.45 (m, 1H), 7.35-7.10 (m, 8H), 6.75 (s, 2H), 4.50 (m, 1H), 4.20 (m, 3H), 3.5-3.1 (m, superimposed by H₂O). 2.70 (m, 1H), 2.35 (m 1H), 2.20 (m, 2H), 1.65 (m, 1H).

### Example 26

### [2-Oxo-1-(2-oxo-2-{[1-(1,4,6,6-tetrahydropyrimidine-2-ylamino)pentyl]amino}ethyl)-2,3,4,5-tetrahydro-1H-1-benzazepine-5-yl]acetic acid

Utilization of N¹-(1,4,5,6-tetrahydropyrimidine-2-yl)pentane-1,5-diamine (hydrochloride) (26) as educt; 40 mg were obtained after MPLC; ESI-MS [M+H⁺]: 444.9.

### Example 27

### {2-Oxo-1-[2-oxo-2-({[4-(pyridine-2-ylamino)cyclohexyl]methyl}amino)ethyl]-2,3,4,5-tetrahydro-1H-1-benzazepine-5-yl}acetic acid

Utilization of N-[4-(aminomethyl)cyclohexyl]pyridine-2-amine (hydrochloride) (26), purification by means of an elution over a Chromabond-C18-cartridge afforded 85 mg; ESI-MS [M+H⁺]: 465.15.

¹H-NMR (360 MHz, d₆-DMSO) δ ppm: 8.55 (s broad, 1H), 8.05 (m, 1H), 7.90 (d, 1H), 7.85 (m, 1H), 7.35 (m, 2H), 7.25 (m, 2H), 6.95 (d, 1H), 6.80 (m, 1H), 4.40 and 4.20 (each m 1H), 3.55 (m, superimposed by H₂O), 2.95 (m, 2H), 2.70 (m, 2H), 2.35 (m, 1H), 2.15, 1.95 and 1.75 (each m, 2H), 1.60 and 1.40 (each m, 1H), 1.25 (m, 2H), 1.05 (m, 2H).

### Example 28

### Ethyl-{2-oxo-1-[2-oxo-2-({[4-(pyridine-2-ylamino)cyclohexyl]methyl}amino)ethyl]-2,3,4,5-tetrahydro-1H-1-benzazepine-5-yl}acetate

30 ml 4n HCl in dioxane were added to 100 mg {2-oxo-1-[2-oxo-2-({[4-(pyridine-2-ylamino)cyclohexyl]methyl}amino)ethyl]-2,3,4,5-tetrahydro-1H-1-benzazepine-5-yl}acetic acid in 10 ml EtOH and it was stirred for 48 h at RT. Evaporation and elution over a Chromabond-C₈-cartridge afforded 26 mg; ESI-MS [M+H⁺]: 493.25.

### Example 29

### (1-{4-[4-(1H-Benzimidazole-2-ylamino)phenyl]butyl}-2-oxo-2,3,4,5-tetrahydro-1H-1-benzazepine-5-yl)acetic acid

a.) A solution of t-butyl (2-oxo-2,3,4,5-tetrahydro-1H-1-benzazepine-5-yl)acetic acid (2) (0.8 g; 2.91 mmol) in 20 ml DMF was added dropwise at 0° C. to a suspension of 0.169 NaH (60%; deoiled) in 10 ml DMF and stirred for 1 h. A spatula tip full of KI was then added likewise at 0° C., 1 g 1-(4-bromo butyl)-4-nitrobenzene (preparation according to J. Med. Chem. 38, 13 (1995), 2418-2420)—dissolved in 10 ml DMF—was slowly added dropwise and stirred for 2 h at RT. For workup, water was carefully added to the reaction mixture, it was diluted with CH₂Cl₂ and several times extracted with a saturated NaCl-solution. After drying and evaporation, the obtained raw product was purified by chromatography on silica gel (CH₂Cl₂/CH₃OH 2-10%); 0.429 yellow oil, ESI-MS [M+H⁺-Boc]: 397.15.

b.) Hydrogenation of the nitro group in 75 ml CH₃OH with 100 mg 10% Pd on activated carbon under standard conditions afforded 0.36 mg of a bright oil; ESI-MS [M+H⁺]: 423.25.

c.) The build-up of the corresponding aminobenzimidazole was carried out analogously to the preparation of 17 by reaction with thiocarbonyldiimidazole, imidazole and then phenylene diamine. Chromatography on silica gel (CH₂Cl₂/CH₃OH 2-4%) afforded 220 mg of a bright foam; ESI-MS [M+H⁺]: 539.25.

d.) 200 mg tert-Butyl-(1-(4-[4-(1H-benzimidazole-2-ylamino)phenyl]butyl)-2-oxo-2,3,4,5-tetrahydro-1H-1-benzazepine-5-yl)acetate was treated at RT with 10 ml TFA. Evaporation and lyophilizing the obtained oil afforded 213 mg of a solid; ESI-MS [M+H⁺]: 483.25.

### Example 30

### 2-(4-{[{[5-(Carboxymethyl)-2-oxo-2,3,4,5-tetrahydro-1H-1-benzazepine-1-yl]acetyl}(methyl)amino]methyl}anilino)-1H-benzimidazole (trifluoro acetate)

a.) Standard coupling of [5-(2-t.Butoxy-2-oxoethyl)-2-oxo-2,3,4,5-tetrahydro-1H-benzazepine-1-yl]acetic acid (3) (2.2 g; 6.6 mmol) with 1.32 g N-methyl(4-nitrophenyl)methane amine (J. Am. Chem. Soc. 65 (1943), 1984-1990) in 40 ml CH₂Cl₂ under utilization of HATU as a coupling reagent and ethyldiisopropylamine as a base. Subsequent chromatography of the raw product on silica gel (CH₂Cl₂/CH₃OH 2-4%) afforded 2.269 of a slightly yellow oil; ESI-MS [M+H⁺]: 482.03.

¹H-NMR (360 MHz, d₆-DMSO) δ ppm (Rotamers): 8.25/8.20 (d, 2H), 7.60/7.50 (d, 2H), 7.35-7.20 (m, 4H), 4.85-4.5 (m, 4H), 3.05/2.85 (s, 3H), 2.70 (m, 1H), 2.30 (m, 1H), 2.15 (m, 2H), 1.65 (m, 1H), 1.30 (s, 9H).

b.) 5 ml Hydrazine hydrate were added at 60° C. to 1.26 g nitro compound a and 15 mg FeCl₃×6H₂O in 20 ml CH₃OH and for 1 h stirred at 60° C. The mixture was filtered over Celite and the filtrate evaporated. 1.17 g; ESI-MS [M+H⁺]: 452.17.

c.) The build-up of the respective aminobenzimidazole was carried out analogously to the preparation of 17 by reaction with thiocarbonyldiimidazole, imidazole and phenylene diamine, subsequently. Chromatography on silica gel (CH₂Cl₂/CH₃OH 4-7.5%) afforded 0.9 g of a slightly beige foam; ESI-MS [M+H⁺]: 568.25.

d.) Cleavage of the t-butylester with 50 ml TFA and subsequent purification by chromatography on silica gel (CH₂Cl₂/CH₃OH 8-10%) afforded 0.88 g; ESI-MS [M+H⁺]: 512.25.

¹H-NMR (360 MHz, d₆-DMSO) δ ppm (Rotamers): 11.05 (broad, 1H), 7.60-7.20 (m, 1.2H), 4.85 (m, 1H), 4.75-4.45 (m, 3H), 3.65 (m, 1H), 3.20 (s, 3H), 2.70 (m, 2H), 2.35 (m, 1H), 2.20 (m, 2H), 1.65 (m, 1H).

### Example 31

### tert-Butyl-[1-(2-{[4-(1H-benzimidazole-2-ylamino)benzyl]amino}-2-oxoethyl)-2,3,4,5-tetrahydro-1H-1-benzazepine-5-yl]acetate

Coupling of [5-(2-tert-butoxy-2-oxoethyl)-2,3,4,5-tetrahydro-1H-1-benzazepine-1-yl]acetic acid (28) with N-[4-(aminomethyl)phenyl]-1H-benzimidazole-2-amine (hydrochloride) (4) analogously to I and purification by chromatography on silica gel (CH₂Cl₂/CH₃OH 2-3%) afforded 200 mg of a beige foam; ESI-MS [M+H⁺]: 540.25.

### Example 32

### [1-(2-{[4-(1H-Benzimidazole-2-ylamino)benzyl]amino}-2-oxoethyl)-2,3,4,5-tetrahydro-1H-1-benzazepine-5-yl]acetic acid (trifluoro acetate)

TFA-cleavage of tert-butyl-[1-(2-{[4-(1H-benzimidazole-2-ylamino)benzyl]amino}-2-oxoethyl)-2,3,4,5-tetrahydro-1H-1-benzazepine-5-yl]acetate afforded 240 mg; ESI-MS [M+H⁺]: 484.36.

¹H-NMR (360 MHz, d₆-DMSO) δ ppm (Rotamers): 11.10 (s, 1H), 8.30 (t, 1H), 7.5-7.25 (m, 8H), 7.15 (m, 2H), 6.85 (m, 2H), 4.40 (m, 2H), 3.75-3.55 (m, superimposed by H₂O), 3.5 (m, 1H), 3.0 (m, 1H), 2.70 (m, 2H), 1.70 (m, 2H), 1.45 (m, 1H).

### Example 33

### tert-Butyl-{1-[2-({[4-(1H-benzimidazole-2-ylamino)cyclohexyl]methyl}amino)-2-oxoethyl]-2,3,4,5-tetrahydro-1H-1-benzazepine-5-yl}acetate

Preparation analogously to example 31 starting from [5-(2-tert-butoxy-2-oxoethyl)-2,3,4,5-tetrahydro-1H-1-benzazepine-1-yl]acetic acid (28) and trans-N-{[4-(aminomethyl)cyclohexyl]}-1H-benzimidazole-2-amine (dihydrochloride) (11). 410 mg of a white foam; ESI-MS [M+H⁺]: 546.35.

### Example 34

### {1-[2-({[4-(1H-Benzimidazole-2-ylamino)cyclohexyl]methyl}amino)-2-oxoethyl]-2,3,4,5-tetrahydro-1H-1-benzazepine-5-yl}acetic acid (trifluoro acetate)

Analogously to example 32; 420 mg of white solids; ESI-MS [M+H⁺]: 490.47.

¹H-NMR (360 MHz, d₆-DMSO) δ ppm: 9.0 (d, 1H), 7.65 (m, 1H), 7.40 and 7.30 (each m, 2H), 7.15 and 6.85 (each m, 2H), 3.80 (m, 2H), 3.50 (m, 1H), 3.0 (m, 4H), 2.75 (m, 2H), 2.0 (m, 2H), 1.75-1.50 (m, 5H), 1.50-1.20 (m, 5H), 0.95 (m, 2H).

### Example 35

### tert-Butyl-[1-(2-{[5-(1H-benzimidazole-2-ylamino)pentyl]amino}-2-oxoethyl)-2,3,4,6-tetrahydro-1H-1-benzazepine-5-yl]acetate

Preparation analogously to example 31 starting from [5-(2-tert-butoxy-2-oxoethyl)-2,3,4,5-tetrahydro-1H-1-benzazepine-1-yl]acetic acid (28) and N¹-(1H-benzimidazole-2-yl)pentane-1,5-diamine (hydrochloride) (8). 300 mg of a foam; ESI-MS [M+H⁺]: 520.39.

### Example 36

### [1-(2-{[5-(1H-Benzimidazole-2-ylamino)pentyl]amino}-2-oxoethyl)-2,3,4,5-tetrahydro-1H-1-benzazepine-5-yl]acetic acid

Cleavage of the t-butyl ester from example 35 with 15 ml 4nHCl in dioxane afforded 300 mg solid; ESI-MS [M+H⁺]: 464.25.

¹H-NMR (360 MHz, d₆-DMSO) δ ppm: 9.10 (m, 1H), 7.70 (m, 1H), 7.35, 7.25, 7.10 and 6.85 (each m, 2H), 4.75 (m 2H), 3.30, 3.15, 2.95, 2.70 (each m, 2H), 1.65 (m, 5H), 1.45 and 1.30 (each m, 2H).

### Example 37

### tert-Butyl-{1-[2-({[4-(1H-imidazole-2-ylamino)cyclohexyl]methyl}amino)-2-oxoethyl]-2-oxo-2,3,4,5-tetrahydro-1H-1-benzazepine-5-yl}acetate

Preparation analogously to example I under utilization of N-[4-(aminomethyl)cyclohexyl]-1H-imidazole-2-amine (hydrobromide) (29) as educt; chromatography on silica gel (CH₂Cl₂/CH₃OH 4-15%) afforded 530 mg of a solid foam; ESI-MS [M+H⁺]: 510.35.

### Example 38

### {1-[2-({[4-(1H-Imidazole-2-ylamino)cyclohexyl]methyl}amino)-2-oxoethyl]-2-oxo-2,3,4,5-tetrahydro-1H-1-benzazepine-5-yl}acetic acid (acetate)

Cleavage of the t-butyl ester from example 37 mit TFA and subsequent purification by means of chromatography on silica gel (CH₂Cl₂/CH₃OH 10-15%+2% glacial acetic acid) afforded 570 mg; ESI-MS [M+H⁺]: 454.25.

¹H-NMR (360 MHz, d₆-DMSO) δ ppm: 8.0 (broad, 1H), 7.30 and 7.25 (each m, 2H), 6.80 (broad, 1H), 6.70 (s, 2H), 4.40 and 4.20 (each broad, 1H), 4.75-4.25 (superimposed by H₂O), 3.0 (m, 2H), 2.65 (m, 2H), 2.35 (m, 1H), 2.15 (m, 2H), 1.95 (m, 1H), 1.75 (m, 2H), 1.60 and 1.35 (each m, 1H), 1.20 (m, 4H), 0.95 (m, 2H).

### Example 39

### tert-Butyl-(2-oxo-1-{[4-({4-[(pyridine-2-ylamino)methyl]piperidine-1-yl}carbonyl)-1,3-thiazole-2-yl]methyl}-2,3,4,5-tetrahydro-1H-1-benzazepine-5-yl)acetate

Standard coupling of 2-{[5-(2-tert-Butoxy-2-oxoethyl)-2-oxo-2,3,4,5-tetrahydro-1H-1-benzazepine-1-yl]methyl}-1,3-thiazole-4-carbonic acid (32) (0.5 g; 1.2 mmol) with N-(piperidine-4-ylmethyl)pyridine-2-amine (hydrochloride; accessibly from 29) in 15 ml CH₂Cl₂ under utilization of HATU as a coupling reagent and ethyldiisopropylamine as a base. Subsequent chromatography of the raw product on silica gel (CH₂Cl₂/CH₃OH 3-20%) afforded 0.44 g of a slightly yellow oil; ESI-MS [M+H⁺]: 590.35.

### Example 40

### (2-Oxo-1-{[4-({4-[(pyridine-2-ylamino)methyl]piperidine-1-yl}carbonyl)-1,3-thiazole-2-yl]methyl}-2,3,4,5-tetrahydro-1H-1-benzazepine-5-yl)acetic acid

Cleavage of the t-butylester of example 31 with TFA afforded 385 mg; ESI-MS [M+H⁺]: 534.25.

¹H-NMR (360 MHz, d₆-DMSO) δ ppm: 8.05 (s, 1H), 7.90 (m, 2H), 7.55 (m, 1H), 7.35 (m, 1H), 7.25 (m, 2H), 7.05 and 6.80 (each m, 1H), 5.40-5.20 (m, 2H), 4.20 and 4.0 (each m, 1H), 3.25 (m, 2H), 2.95 (m, 1H), 2.80-2.60 (m, 3H), 2.30 (m, 1H), 2.25-2.05 (m, 2H), 1.80 and 1.65 (each m, 2H), 1.20-1.10 (m, 3H).

### Example 41

### tert-Butyl-[1-({4-[({[4-(1H-benzimidazole-2-ylamino)cyclohexyl]methyl}amino) carbonyl]-1,3-thiazole-2-yl}methyl)-2-oxo-2,3,4,6-tetrahydro-1H-1-benzazepine-5-yl]acetate

Analogously to example 29 under utilization of trans-N-{[4-(aminomethyl)cyclohexyl]}-1H-benzimidazole-2-amine (dihydrochloride) (11) as educt; chromatography of the raw product on silica gel (CH₂Cl₂/CH₃OH 5-8%) afforded 350 mg of a bright yellow oil; ESI-MS [M+H⁺]: 643.45.

### Example 42

### [1-({4-[({[4-(1H-Benzimidazole-2-ylamino)cyclohexyl]methyl}amino)carbonyl]-1,3-thiazole-2-yl}methyl)-2-oxo-2,3,4,6-tetrahydro-1H-1-benzazepine-5-yl]acetic acid

Cleavage of the t-butylester of example 31 and purification by elution over Chromabond-C18 (acetonitrile/H₂O 10-80%+0.1% glacial acetic acid) afforded 300 mg; ESI-MS [M+H⁺]: 587.35.

¹H-NMR (360 MHz, d₆-DMSO) δ ppm: 8.40 (broad, 1H), 8.20 (m, 1H), 8.20 (s, 1H), 7.35-7.20 (m, 5H), 7.15 (m, 2H), 5.40 and 5.25 (each d, 1H), 3.15 (m, superimposed by H₂O), 2.80-2.60 (m, 2H), 2.35 (m, 1H), 2.20 (m, 2H), 2.05 and 1.80 (each m, 2H), 1.65, 1.30 and 1.15 (each m, 2H).

### Example 43

### tert-Butyl-(1-{[4 ({4-[(1H-benzimidazole-2-ylamino)methyl]piperidine-1-yl}carbonyl)-1,3-thiazole-2-yl]methyl}-2-oxo-2,3,4,5-tetrahydro-1H-1-benzazepine-5-yl)acetate

430 mg were obtained analogously to example 29 under utilization of N-(piperidine-4-ylmethyl)-1H-benzimidazole-2-amine (trifluoro acetate) (17) and subsequent purification; ESI-MS [M+H⁺]: 629.45.

### Example 44

### (1-{[4-({4-[(1H-Benzimidazole-2-ylamino)methyl]piperidine-1-yl}carbonyl)-1,3-thiazole-2-yl]methyl}-2-oxo-2,3,4,5-tetrahydro-1H-1-benzazepine-5-yl)acetic acid

Cleavage of the t-butylester of example 43 and purification by elution over Chromabond-C18 (acetonitrile/H₂O 10-80%+0.1% glacial acetic acid) afforded 340 mg; ESI-MS [M+H⁺]: 573.35.

¹H-NMR (360 MHz, d-DMSO) δ ppm: 9.20 (m, 1H), 7.95 (s, 1H), 7.45 (m, 1H), 7.40 (m, 2H), 7.30-7.15 (m, 5H), 5.35-5.10 (m, 2H), 4.45 (m, 1H), 4.0-3.25 (m, superimposed by H₂O), 2.95 (m, 1H), 2.8-2.60 (m, 2H), 2.30 and 2.20 (each m, 1H), 2.0-1.75 (m, 3H), 1.70-1.50 (m, 2H), 1.20 (m, 3H).

### Example 45

### tert-Butyl-{2-oxo-1-[(4-{[({4-[(pyridine-2-ylamino)methyl]thiene-2-yl}methyl)amino]carbonyl}-1,3-thiazole-2-yl)methyl]-2,3,4,5-tetrahydro-1H-1-benzazepine-5-yl}acetate

330 mg were obtained analogously to example 39 under utilization of N-{[5-(aminomethyl)thiene-3-yl]methyl}pyridine-2-amine (trifluoro acetate) (18) and purification; ESI-MS [M+H⁺]: 618.25.

### Example 46

### {2-Oxo-1-[(4-{([{4-[(pyridine-2-ylamino)methyl]thiene-2-yl}methyl)amino]carbonyl}-1,3-thiazole-2-yl)methyl]-2,3,4,5-tetrahydro-1H-1-benzazepine-5-yl}acetic acid

Cleavage of the t-butylesters of example 45 and purification by means of MPLC afforded 150 mg; ESI-MS [M+H⁺]: 562.25.

¹H-NMR (360 MHz, d₆-DMSO) δ ppm: 8.80 (m, 1H), 8.20 (m, 1H), 8.0 (d, 1H), 7.50 (d, 1H), 7.30-7.20 (m, 4H), 7.10 and 6.95 (each s, 1H), 6.85 (m, 1H), 6.50 (m, 2H), 5.40 and 5.20 (each d, 1H), 4.55 and 4.35 (each m, 2H), 2.80-2.55 (m, 2H), 2.30 (m, 1H), 2.20 (m, 2H), 1.65 (m, 1H).

### Example 47

### tert-Butyl-[1-({4-[4-(1H-benzimidazole-2-ylamino)phenyl]-1,3-thiazole-2-yl}methyl)-2-oxo-2,3,4,5-tetrahydro-1H-1-benzazepine-5-yl]acetate

a.) A mixture of 1.5 g tert-butyl-[1-(2-amino-2-thioxoethyl)-2-oxo-2,3,4,5-tetrahydro-1H-1-benzazepine-5-yl]acetate, 1.36 g 2-bromo-4-nitroacetophenone and 0.65 g KHCO₃ in 30 ml dioxane were stirred for 12 h at RT. Workup analogously to building block 30c and mixing of the raw product with n-pentane afforded 2.1 g of a brown solid; ESI-MS [M+H⁺]: 494.15.

¹H-NMR (360 MHz, d₆-DMSO) δ ppm: 8.40 (s, 1H), 8.30 and 8.20 (each m, 2H), 7.60 and 7.35 (each m, 1H), 7.25 (m, 2H), 5.50 and 5.30 (each d, 1H), 2.70 (m, 2H), 2.30 (m, 1H), 2.20 (m, 3H), 1.65 (m, 1H), 1.25 (s, 9H).

b.) Reduction of the nitro group analogously to example 30b (1.6 g; ESI-MS [M+H⁺]: 464.15) and build-up of the aminobenzimidazole as already described afforded after chromatography on silica gel (CH₂Cl₂/CH₃OH 24%) 0.58 g of a slightly yellow foam; ESI-MS [M+H⁺]: 614.35.

### Example 48

### [1-({4-[4-(1H-Benzimidazole-2-ylamino)phenyl]-1,3-thiazole-2-yl}methyl)-2-oxo-2,3,4,5-tetrahydro-1H-1-benzazepine-5-yl]acetic acid

Cleavage of the t-butylester of example XXXXVII and purification by means of MPLC afforded 40 mg; ESI-MS [M+H⁺]: 524.25.

¹H-NMR (360 MHz, d₆-DMSO) δ ppm: 11.95 and 9.60 (each broad, 1H), 7.85-7.75 (m, 4H), 7.65 (m, 1H), 7.40-7.25 (m, 5H), 7.0 (m, 2H), 5.40 and 5.30 (each d, 1H), 3.50 (m, 1H), 2.75 (m, 1H), 2.45 (m, 2H), 2.20 (m, 2H), 1.70 (m, 1H).

### Example 49

### tert-Butyl (1-{2-[(4{[amino(imino)methyl]amino}benzyl)amino]-2-oxoethyl}-2-oxo-2,3,4,5-tetrahydro-1H-1-benzazepine-5-yl)acetic acid

Ethyldiisopropylamine (0.42 mmol, 54.50 mg) and HATU (0.50 mmol, 190.11 mg) were added at 0° C. to a solution of [5-(2-t-butoxy-2-oxoethyl)-2-oxo-2,3,4,5-tetrahydro-1H-benzazepine-1-yl]acetic acid (3) (0.42 mmol, 140.59 mg) in CH₂Cl₂ (10 ml), the mixture was stirred for 1 h at 0° C. and [4-(aminomethyl)phenyl]guanidine (bihydrochloride) (46) (0.42 mmol, 100 mg), ethyldiisopropylamine (0.63 mmol, 81.76 mg) added. The mixture was stirred for 1 h at 0° C. and overnight at RT. After evaporation, the residue was taken up in CH₂Cl₂/water, washed with watery NaHCO₃—, 5%-citric acid- and finally with watery saturated NaCl-solution. Evaporation and chromatography on silica gel (CH₂Cl₂/CH₃OH 0-100%) afforded 6.0 mg target product; ESI-MS [M+H⁺]: 426.1, 425.1.

### Example 50

### {4-[({[5-(Carboxymethyl)-2-oxo-2,3,4,5-tetrahydro-1H-1-benzazepine-1-yl]acetyl}amino)methyl]anilino}(imino)methaneamine (trifluoro acetate)

tert-Butyl-({2-[(4{[amino(imino)methyl]amino}benzyl)amino]-2-oxoethyl}-2-oxo-2,3,4,5-tetrahydro-1H-1-benzazepine-5-yl)acetic acid (example 49, 0.01 mmol, 6 mg) was dissolved in 5 ml CH₂Cl₂, TFA (1.25 mmol, 142.53 mg) added at 0° C. and stirred overnight at room temperature. After evaporation, the residue was taken up in CHCl₃/water and the watery phase washed with CHCl₃; evaporation of the watery phase affords 4.8 mg target product; ESI-MS [M+H⁺]=425.1.

### Example 51

### tert-Butyl [1-(2-{[(6-amino-2-pyridinyl)methyl]amino}-2-oxoethyl)-2-oxo-2,3,4,5-tetrahydro-1H-1-benzazepine-5-yl]acetic acid

Analogously to example 49, under utilization of 2-ammonio-6-(ammonio methyl)pyridinium trichloride (33) (0.30 mmol, 69.76 mg) and following purification and subsequent purification (chromatography; CH₂Cl₂/MeOH 0-100%) afforded 120 mg of the target product; ESI-MS: [M+H⁺]=439.28, 383.36.

### Example 52

### [1-(2-{([(6-Amino-2-pyridinyl)methyl]amino}-2-oxoethyl)-2-oxo-2,3,4,5-tetrahydro-1H-1-benzazepine-5-yl]acetic acid (hydrochloride)

Cleavage of the t-butylester, chromatography of the raw product (CHCl₃/MeOH 0-5%) and ester interchange with HCl in diethyl ether afforded 15.0 mg; the target product as hydrochloride; ESI-MS [M+H⁺]=383.1.

### Example 53

### tert-Butyl [1-(2-{[4-(1H-benzimidazole-2-ylamino)benzyl]amino}ethyl)-2-oxo-2,3,4,5-tetrahydro-1H-1-benzazepine-5-yl]acetic acid

2 Drops of a solution of HCl in diethylether were added to N-[4-(aminomethyl)phenyl]-1H-benzimidazole-2-amine (hydrochloride) (4) (0.63 mmol, 196.10 mg) in methanol (40 mL) and stirred for 5 min. at RT. tert-Butyl. [2-oxo-1-(2-oxoethyl)-2,3,4,5-tetrahydro-1H-1-benzazepine-5-yl]acetic acid (37, 0.63 mmol, 0.20 g) was added, NaBH₃CN (3 mmol, 188.5 mg) portionwise added after 10 min. and the mixture stirred for 17 h at RT. After evaporation, the residue was taken up in ethyl acetate and washed with NaHCO₃— (pH 8-9), saturated NaCl— (1×) and a 10% FeSO₄-solution (2×). Chromatography (CHCl₃/MeOH 0-5%) afforded 84 mg of the target product; ESI-MS [M+H⁺]=540.24, 270.75.

### Example 54

### [1-(2-{[4-(1H-Benzimidazole-2-ylamino)benzyl]amino}ethyl)-2-oxo-2,3,4,6-tetrahydro-1H-1-benzazepine-5-yl]acetic acid

Cleavage of the t-butylester and chromatography (CHCl₃/MeOH 0-100%) afforded 4 mg of the target product; ESI-MS [M+H⁺]=484.1.

### Example 55

### tert-Butyl [1-(2-{[(4-{[(benzylamino)carbonyl]amino}cyclohexyl)methyl]amino}-2-oxoethyl)-2-oxo-2,3,4,5-tetrahydro-1H-1-benzazepine-5-yl]acetic acid

Analogously to example 49 under utilization of N-[4-(aminomethyl)cyclohexyl]-N′-benzyl urea (34) (0.50 mmol, 130 mg) afforded 320 mg; ESI-MS [M+H⁺]=577.11.

### Example 56

### [1-(2-{[(4-{[(Benzylamino)carbonyl]amino}cyclohexyl)methyl]amino}-2-oxoethyl)-2-oxo-2,3,4,5-tetrahydro-1H-1-benzazepine-5-yl]acetic acid

Analogous cleavage of the t-butylester afforded a raw product which after evaporation was distributed between ethyl acetate/water. The watery phase was then adjusted to pH 10 with 0.1n NaOH and extracted with ethyl acetate. It was adjusted then with 1nN HCl to pH4, extracted with CH₂Cl₂, washed and evaporated; 80 mg; ESI-MS [M+H⁺]=521.3.

### Example 57

### tert-Butyl [1-(2-{[5-(1H-benzimidazole-2-ylamino)pentyl]amino}ethyl)-2-oxo-2,3,4,5-tetrahydro-1H-1-benzazepine-5-yl]acetic acid

Preparation analogously to example 53 under utilization of N¹-(1H-benzimidazole-2-yl)pentane-1,5-diamine (hydrochloride) (8) (0.63 mmol, 183.47 mg) and tert-butyl [2-oxo-1-(2-oxoethyl)-2,3,4,5-tetrahydro-1H-1-benzazepine-5-yl]acetic acid (37, 0.63 mmol, 0.20 g) afforded 50.0 mg of the target product; ESI-MS [M+H⁺]=520.41, 260.79.

### Example 58

### [1-(2-{[5-(1H-Benzimidazole-2-ylamino)pentyl]amino}ethyl)-2-oxo-2,3,4,5-tetrahydro-1H-1-benzazepine-5-yl]acetic acid (trifluoro acetate)

tert-Butyl [1-(2-{[5-(1H-benzimidazole-2-ylamino)pentyl]amino}ethyl)-2-oxo-2,3,4,5-tetrahydro-1H-1-benzazepine-5-yl]acetic acid (example 57, 0.04 mmol, 20.00 mg) was dissolved in 5 ml CH₂Cl₂, TFA (65.34 mmol, 7.45 g) added at 0° C. and stirred overnight at room temperature. Evaporation of the reaction mixture, chromatography (CHCl₃/MeOH 0-100%) afforded 10 mg of the target product; ESI-MS [M+H⁺]=464.25.

### Example 59

### tert-Butyl {2-oxo-1-[2-({[4-({4-[(1Z)-1-propenyl]-5-vinyl-1H-imidazole-2-yl}amino)cyclohexyl]methyl}amino)ethyl]-2,3,4,6-tetrahydro-1H-1-benzazepine-5-yl}acetic acid

Preparation analogously to example 53 under utilization of ethyldiisopropylamine and EDCI*HCl starting from trans-N-[[4-(aminomethyl)cyclohexyl]-1H-benzimidazole-2-amine (dihydrochloride) (11) (0.63 mmol, 0.209) afforded 84 mg; ESI-MS [M+H⁺]=546.32, 273.79.

### Example 60

### {1-[2-({[4-(1H-Benzimidazole-2-ylamino)cyclohexyl]methyl}amino)ethyl]-2-oxo-2,3,4,5-tetrahydro-1H-1-benzazepine-5-yl}acetic acid (trifluoro acetate)

tert-Butyl {2-oxo-1-[2-({[4-({4-[(1Z)-1-propenyl]-5-vinyl-1H-imidazole-2-yl}amino)cyclohexyl]methyl}amino)ethyl]-2,3,4,5-tetrahydro-1H-1-benzazepine-5-yl}acetic acid (example 59, 0.01 mmol, 8 mg) was dissolved in 5 ml CH₂Cl₂, TFA (65.34 mmol, 7.45 g) added at 0° C. and stirred overnight at room temperature. The reaction mixture was evaporated, the watery phase washed with a 3:1 mixture CHCl₃/EtOH. Evaporation of the watery phase afforded 6.0 mg of the target product; ESI-MS [M+H⁺]=490.25.

### Example 61

### [1-(2-{[4-(1H-Benzimidazole-2-ylamino)benzyl]amino}-2-oxoethyl)-7-chloro-2-oxo-2,3,4,5-tetrahydro-1H-1-benzazepine-5-yl]acetic acid (trifluoro acetate)

Coupling analogously to example 49 starting from N-[4-(aminomethyl)phenyl]-1H-benzimidazole-2-amine (hydrochloride) (4) (1.70 mmol, 467.08 mg) and [5-(2-tert-butoxy-2-oxoethyl)-7-chloro-2-oxo-2,3,4,5-tetrahydro-1H-1-benzazepine-1-yl]acetic acid (45) (1.63 mmol, 600 mg). TFA (1.73 mmol, 197.72 mg) was added at 0° C. to the obtained raw product and it was stirred overnight at room temperature. After evaporation, the residue was distributed between ethyl acetate and water, the watery phase adjusted to pH 10 with 2n NaOH and extracted with ethyl acetate; purification by means of MPLC afforded 90 mg of the target product as TFA-salt; ESI-MS [M+K⁺]=570.2, 534.2, 532.2, 266.5, 101.1.

### Example 62

### [1-(2-{[5-(1H-Benzimidazole-2-ylamino)pentyl]amino}-2-oxoethyl)-7-chloro-2-oxo-2,3,4,5-tetrahydro-1H-1-benzazepine-5-yl]acetic acid (trifluoro acetate)

Coupling analogously to example 49 starting from N¹-(1H-benzimidazole-2-yl)pentane-1,5-diamine (hydrochloride) (8) (1.7 mmol, 433.10 mg) and [5-(2-tert-butoxy-2-oxoethyl)-7-chloro-2-oxo-2,3,4,5-tetrahydro-1H-1-benzazepine-1-yl]acetic acid (45) (1.63 mmol, 600.00 mg). Treatment of the raw product with TFA analogously to example 61 afforded 48 mg as a TFA-salt; ESI-MS: 514.2, 512.2, 101.2.

### Example 63

### [1-(2-{[4-(1H-Benzimidazole-2-ylamino)butyl]amino}-2-oxoethyl)-7-chloro-2-oxo-2,3,4,5-tetrahydro-1H-1-benzazepine-5-yl]acetic acid

Coupling analogously to example 49 starting from N¹-(1H-benzimidazole-2-yl)butane-1,4-diamine (trifluoro acetate) (9) (1.7 mmol, 541.1 mg) and [5-(2-tert-butoxy-2-oxoethyl)-7-chloro-2-oxo-2,3,4,5-tetrahydro-1H-1-benzazepine-1-yl]acetic acid (45) (1.63 mmol, 600 mg). Treatment of the raw product with TFA analogously to example 61 afforded 15 mg as a TFA-salt; ESI-MS: 536.2, 500.1, 498.2, 105.1, 101.2.

### Example 64

### [7-Chloro-1-(2-{[5-(4,5-dihydro-1H-imidazole-2-ylamino)pentyl]amino}-2-oxoethyl)-2-oxo-2,3,4,5-tetrahydro-1H-1-benzazepine-5-yl]acetic acid (trifluoro acetate)

Coupling analogously to example 49 starting from N¹-(4,5-dihydro-1H-imidazole-2-yl)pentane-1,5-diamine (hydrochloride) (23) (1.70 mmol, 351.43 mg) and [5-(2-tert-butoxy-2-oxoethyl)-7-chloro-2-oxo-2,3,4,5-tetrahydro-1H-1-benzazepine-1-yl]acetic acid (45) (1.63 mmol, 600 mg). Treatment of the raw product with TFA analogously to example 61 afforded 85 mg as a TFA-salt; ESI-MS: 464.15.

### Example 65

### tert-Butyl 7-[4-({[5-(2-tert-butoxy-2-oxoethyl)-2-oxo-2,3,4,5-tetrahydro-1H-1-benzazepine-1-yl]acetyl}amino)butyl]-3,4-dihydro[1,8]naphthyridine-1(2H)-carboxylate

Ethyldiisopropylamine (2.08 mmol, 268.41 mg) and EDCI*HCl (0.78 mmol, 150.44 mg) were added at 0° C. to a solution of [5-(2-t.butoxy-2-oxoethyl)-2-oxo-2,3,4,5-tetrahydro-1H-benzazepine-1-yl]acetic acid (3) (0.46 mmol, 0.15 g) in CH₃CN (45 ml). After 1 h at 0° C., 7-(4-aminobutyl)-1,2,3,4-tetrahydro[1,8]naphthyridine (bitrifluoro acetate) (35) (0.46 mmol, 0.2 g) was added, stirred for 1 h at 0° C. and overnight at RT. Since by chromatography a purification could not be achieved, the obtained was reacted to the corresponding Boc-derivative according to standard methods. Chromatography (heptane/CH₂Cl₂ 0-100%, CH₂Cl₂/MeOH 0-100%) afforded 15 mg of the target product (about 95% purity); ESI-MS [M+Na⁺]: 643.5, 622.5, [M+H⁺]=621.5.

### Example 66

### 7-[4-({[5-(Carboxymethyl)-2-oxo-2,3,4,5-tetrahydro-1H-1-benzazepine-1-yl]acetyl}amino)butyl]-1,2,3,4-tetrahydro[1,8]naphthyridine (trifluoro acetate)

tert-Butyl 7-[4-({[5-(2-tert-butoxy-2-oxoethyl)-2-oxo-2,3,4,5-tetrahydro-1H-1-benzazepine-1-yl]acetyl}amino)butyl]-3,4-dihydro[1,8]naphthyridine-1(2H)-carboxylate (example 65; 0.02 mmol, 15 mg) was dissolved in 2 ml CH₂Cl₂, TFA (0.53 mmol, 60.7 mg) added at 0° C. and stirred overnight at RT. Usual workup afforded 9.3 mg of the target product as TFA-salt; ESI-MS: 466.2, [M+H⁺]=465.2, 233.3.

### Example 67

### N-{4-[({[5-(Carboxymethyl)-7-chloro-2-oxo-2,3,4,5-tetrahydro-1H-1-benzazepine-1-yl]acetyl}amino)methyl]cyclohexyl})-1H-benzimidazole (acetate)

Coupling analogously to example 49 starting from trans-N-[4-(aminomethyl)cyclohexyl]-1H-benzimidazole-2-amine (dihydrochloride) (11) (2.99 mmol, 948.78 mg) and [5-(2-tert-butoxy-2-oxoethyl) 7 chloro-2-oxo-2,3,4,5-tetrahydro-1H-1-benzazepine-1-yl]acetic acid (45) (2.72 mmol, 1.00 g) afforded 760 mg raw product. Treatment of the raw product with TFA analogously to example 61 and purification of the raw product by means of MPLC afforded 400 mg; ESI-MS: [M+H⁺]=540.3, 538.25, 269.6, 101.1.

### Example 68

### Ethyl-{1-[2-({[4-(1H-benzimidazole-2-ylamino)cyclohexyl]methyl}amino)-2-oxoethyl]-2,3,4,5-tetrahydro-1H-1-benzazepine-5-yl}acetate

15 ml HCl in diethyl ether (saturated at 0° C.) were added to 300 mg {1-[2-({[4-(1H-benzimidazole-2-ylamino)cyclohexyl]methyl}amino)-2-oxoethyl]-2,3,4,5-tetrahydro-1H-1-1-benzazepine-5-yl}acetic acid in 30 ml ethyl alcohol and allowed to stand for 3 days at room temperature. The mixture was evaporated, the remaining residue purified by chromatography on silica gel (CH₂Cl₂/CH₃OH 5%+1% glacial acetic acid) and lyophilized. 230 mg; ESI-MS: [M+H⁺]=518.35.

### Example 69

### {1-[2-({[4-(1H-Benzimidazole-2-ylamino)cyclohexyl]methyl}amino)-2-oxoethyl]-7,8-dimethoxy-2-oxo-2,3,4,5-tetrahydro-1H-1-benzazepine-5-yl}acetic acid (acetate)

Standard coupling of [5-(2-tert-butoxy-2-oxoethyl)-7,8-dimethoxy-2-oxo-2,3,4,5-tetrahydro-1H-1-benzazepine-1-yl]acetate (building block 49) (1 g; 2.54 mmol) with trans-N-{[4-(aminomethyl)cyclohexyl]}-1H-benzimidazole-2-amine (dihydrochloride) (11); after chromatography on silica gel (CH₂Cl₂/CH₃OH 5-7%), 1.03 g were isolated as a white glass; ESI-MS: [M+H⁺]=620.35. Cleavage of the ester under utilization of TFA and subsequent purification of the raw product by chromatography on silica gel (CH₂Cl₂/CH₃OH 10%+2% glacial acetic acid) afforded 0.8 g of the target product; ESI-MS: [M+H⁺]=564.25.

### Example 70

### [1-(2-{[4-(1H-Benzimidazole-2-ylamino)benzyl]amino}-2-oxoethyl)-7,8-dimethoxy-2-oxo-2,3,4,5-tetrahydro-1H-1-benzazepine-5-yl]acetic acid

Standard coupling of [5-(2-tert-butoxy-2-oxoethyl)-7,8-dimethoxy-2-oxo-2,3,4,5-tetrahydro-1H-1-benzazepine-1-yl]acetate (building block 49) (0.5 g; 1.27 mmol) with N-[4-(aminomethyl)phenyl]-1H-benzimidazole-2-amine (hydrochloride) (4); after chromatography on silica gel (CH₂Cl₂/CH₃OH 3-5%) 0.67 g were isolated as a foam; ESI-MS: [M+H⁺]=614.35. Cleavage of the ester under utilization of TFA and lyophilization of the obtained product afforded 0.61 g; ESI-MS: [M+H⁺]=558.35.

### Example 80

### {5-[2-({[4-(1H-Benzimidazole-2-yl)cyclohexyl]methyl}amino)-2-oxoethyl]-2-oxo-2,3-dihydro-1H-1-benzazepine-1-yl}acetic acid

0.4 g (1.4 mmol) [1-(2-Methoxy-2-oxoethyl)-2-oxo-2,3-dihydro-1H-1-benzazepine-5-yl]acetic acid (50) and 0.37 g (1.4 mmol) [4-(1H-benzimidazole-2-yl)cyclohexyl]methane amine (hydrochloride) (51) were reacted analogously to example I, the reaction product then purified by means of preparative thick-layer chromatography (eluent: CH₂Cl₂/CH₃OH/conc. NH₃ 45/5/0.2) and then the methyl ester cleaved with 0.8 ml 1n NaOH in 8 ml dioxane at room temperature. After neutralization with 1n HCl, evaporation of the solvent, extraction with ethyl acetate and drying with Na₂SO₄, the raw product was purified by chromatography (CH₂Cl₂/CH₃OH/50% glacial acetic acid 20/5/1). After lyophilization, 0.22 g remained as a white amorphous residue; ESI-MS: [M+H⁺]=487.

### Example 81

### [5-(2-{[5-(1H-Benzimidazole-2-ylamino)pentyl]amino}-2-oxoethyl)-2-oxo-2,3-dihydro-1H-1-benzazepine-1-yl]acetic acid

Analogously to example I 0.4 g (1.4 mmol) [1-(2-methoxy-2-oxoethyl)-2-oxo-2,3-dihydro-1H-1-benzazepine-5-yl]acetic acid (50) and 0.36 g (1.4 mmol) N¹-(1H-benzimidazole-2-yl)pentane-1,5-diamine (hydrochloride) (8) were reacted. Purification of the raw product, alkaline saponification of the methyl ester and purification of the end stage was conducted analogously to example 80. 0.3 g of a white amorphous residue; ESI-MS: [M+H⁺]=476.

### Example 82

### [5-(2-{4-[(1H-Benzimidazole-2-ylamino)methyl]piperidine-1-yl}-2-oxoethyl)-2-oxo-2,3-dihydro-1H-1H-benzazepine-1-yl]acetic acid

Analogously to example I 0.49 (1.4 mmol) [1-(2-methoxy-2-oxoethyl)-2-oxo-2,3-dihydro-1H-1-benzazepine-5-yl]acetic acid (50) was reacted with 0.63 g (1.4 mmol) N-(piperidine-4-ylmethyl)-1H-benzimidazole-2-amine (trifluoro acetate) (17). After purification, alkaline ester hydrolysis and chromatographic purification of the end product (CH₂Cl₂/CH₃OH/50% glacial acetic acid 20/5/1) 0.3 g of a white amorphous powder were obtained; ESI-MS: [M+H⁺]=488.

### Example 83

### [5-(2-{4-[(1H-Benzimidazole-2-ylamino)methyl]piperidine-1-yl}-2-oxoethyl)-2-oxo 2,3-dihydro-1H-1-benzazepine-1-yl]acetic acid

Analogously to example I 0.4 g (1.4 mmol) [1-(2-methoxy-2-oxoethyl)-2-oxo-2,3-dihydro-1H-1-benzazepine-5-yl]acetic acid (50) were reacted with 0.43 g trans-N-J[4-(aminomethyl)cyclohexyl]₁H-benzimidazole-2-amine (dihydrochloride) (11). After purification of the ester (eluent: CH₂Cl₂/CH₃OH/25% NH₃ 45/5/0.2), alkaline ester hydrolysis and chromatographic purification of the end product (CH₂Cl₂/CH₃OH/50% glacial acetic acid 20/5/1) 0.18 g of a white amorphous powder were obtained; ESI-MS: [M+H⁺]=502.

### Example 84

### [5-(2-{[5-H-Benzimidazole-2-ylamino)pentyl]amino}-2-oxoethyl)-4-oxo-2,3,4,5-tetrahydro-1H-1,5-benzodiazepine-1-yl]acetic acid (hydrochloride)

Analogously to example I 0.65 g (2 mmol) [5-(2-tert-butoxy-2-oxoethyl)-2-oxo-2,3,4,5-tetrahydro-1H-1,5-benzodiazepine-1-yl]acetic acid (54) were reacted with N¹-(1H-benzimidazole-2-yl)pentane-1,5-diamine (hydrochloride) (8), the reaction product purified by means of preparative thick-layer chromatography (eluent: CH₂Cl₂/CH₃OH/conc. NH₃ 45/5/0.2); ESI-MS: [M+H⁺]=535. Then the tert-butyl ester was cleaved with 4n HCl in dioxane, and after chromatographic purification 40 mg of a white amorphous powder were isolated; ESI-MS: [M+H⁺]=479.

### Example 85

### {5-[2-({[4-(1H-Benzimidazole-2-ylamino)cyclohexyl]methyl}amino)-2-oxoethyl]-4-oxo-2,3,4,5-tetrahydro-1H-1,5-benzodiazepine-1-yl}acetic acid (hydrochloride)

Preparation and purification was conducted analogously to example 84 by reaction with trans-N-{[4-(aminomethyl)cyclohexyl]}-1H-benzimidazole-2-amine (dihydrochloride) (11); ester stage ESI-MS: [M+H⁺]=561. Purification after ester cleavage was conducted by means of preparative thick-layer chromatography (CH₂Cl₂/CH₃OH/50% glacial acetic acid 12/3/1); 0.35 g of a white amorphous powder; ESI-MS: [M+H⁺]=505.

### Example 86

### [5-(2-{[4-(1H-Benzimidazole-2-ylamino)benzyl]amino}-2-oxoethyl)-4-oxo-2,3,4,5-tetrahydro-1H-1,5-benzodiazepine-1-yl]acetic acid (hydrochloride)

Preparation and purification was conducted analogously to example 84 by reaction with N-[4-(aminomethyl)phenyl]-1H-benzimidazole-2-amine (hydrochloride) (4); ester stage ESI-MS: [M+H⁺]=555; target product: 0.4 g of a white amorphous powder; ESI-MS: [M+H⁺]=499.

### Example 87

### [5-(2-{[4-(1H-Benzimidazole-2-ylamino)benzyl]amino}-2-oxoethyl)-2-oxo-2,3,4,5-tetrahydro-1H-1,5-benzodiazepine-1-yl]acetic acid

Analogously to example I 1.2 g (3.6 mmol) [5-(2-tert-butoxy-2-oxoethyl)-4-oxo-2,3,4,5-tetrahydro-1H-1,5-benzodiazepine-1-yl]acetic acid (55) was reacted with 0.86 g (3.6 mmol) N-[4-(aminomethyl)phenyl]-1H-benzimidazole-2-amine (hydrochloride) (4), the reaction product purified by means of preparative thick-layer chromatography (eluent: CH₂Cl₂/CH₃OH/conc. NH₃ 45/5/0.1); ESI-MS: [M+H⁺]=: 555. Subsequent cleavage of the tert-butylester with 4n HCl in dioxane afforded 0.8 g of a white amorphous powder; ESI-MS [M+H⁺]=499.

### Example 88

### {5-[2-({[4-(1H-Benzimidazole-2-ylamino)cyclohexyl]methyl}amino)-2-oxoethyl]-2-oxo-2,3,4,5-tetrahydro-1H-1,5-benzodiazepine-1-yl}acetic acid

Preparation and purification analogously to example 87; ester stage ESI-MS: [M+H⁺]=561; 0.9 g of the target product were obtained as a white amorphous powder; ESI-MS [M+H⁺]=505.

### Example 89

### (1-{2-[4-(1H-Benzimidazole-2-yl)piperidine-1-yl]-2-oxoethyl}-2-oxo-2,3,4,5-tetrahydro-1H-1-benzazepine-5-yl)acetic acid (acetate)

Analogously to example I 0.5 g (1.5 mmol) [5-(2-t-butoxy-2-oxoethyl)-2-oxo-2,3,4,5-tetrahydro-1H-benzazepine-1-yl]acetic acid (3) and 0.36 g (1.5 mmol) 2-(4-piperidinyl)-1H-benzimidazole (J. Heterocycl. Chem. 1989, 26, 541) were reacted, purified by means of preparative thick-layer chromatography (eluent CH₂Cl₂/CH₃OH/conc. NH₃ 45/5/0.2); ESI-MS: [M+H⁺]=517. Subsequent cleavage of the tert-butyl ester with 4n HCl in a mixture of dioxane/glacial acetic acid and chromatographic purification afforded 0.259 of a white amorphous powder; ESI-MS [M+H⁺]=: 461.

### Example 90

### (1-{2-[[3-(1H-Benzimidazole-2-yl)propyl](methyl)amino]-2-oxoethyl}-2-oxo-2,3,4,6-tetrahydro-1H-1-benzazepine-5-yl)acetic acid (hydrochloride)

Preparation analogously to example I by reaction with N-[3-(1H-benzimidazole-2-yl)propyl]-N-methylamine (WO 9849148); after chromatographic purification 0.69 were obtained as a white amorphous powder; ESI-MS [M+H⁺]=: 449.

### Example 91

### (1-{2-[4-(1H-Benzimidazole-2-ylamino)piperidine-1-yl]-2-oxoethyl}-2-oxo-2,3,4,6-tetrahydro-1H-1-benzazepine-5-yl)acetic acid

Preparation analogously to example I by reaction with N-piperidine-4-yl-1H-benzimidazole-2-amine (J. Med. Chem. 1985, 28, 1925); after chromatographic purification 0.45 g were obtained as a white amorphous powder; ESI-MS [M+H⁺]=: 476.

### Example 92

### [5-(2-{[4-(1H-Benzimidazole-2-ylamino)benzyl]amino}-2-oxoethyl)-2-oxo-2,3,4,5-tetrahydro-1H-1-benzazepine-1-yl]acetic acid

Analogously to example I 0.4 g (1.4 mmol) [1-(2-methoxy-2-oxoethyl)-2-oxo-2,3,4,5-tetrahydro-1H-1-benzazepine-5-yl]acetic acid (56) and 0.33 g (1.4 mmol) N-[4-(aminomethyl)phenyl]-1H-benzimidazole-2-amine (hydrochloride) (4) were reacted and purified by means of preparative thick-layer chromatography (eluent: CH₂Cl₂/CH₃OH/conc. NH₃ 45/5/0.2); ESI-MS: [M+H⁺]=512. Subsequent cleavage of the methyl ester with 1n NaOH in dioxane and chromatographic purification of the residue (CH₂Cl₂/CH₃OH/50% glacial acetic acid 20/5/1) afforded 0.15 g, isolated as a white amorphous powder; ESI-MS [M+H⁺]=498.

### Example 93

### {5-[2-({[4-(1H-Benzimidazole-2-yl)cyclohexyl]methyl}amino)-2-oxoethyl]-2-oxo-2,3,4,5-tetrahydro-1H-1-benzazepine-1-yl}acetic acid

Preparation and purification analogously to example 92; ester stage ESI-MS [M+H⁺]=503; chromatographic purification after cleavage of the ester afforded 0.3 g of the target product as a white amorphous powder; ESI-MS [M+H⁺]=489.

### Example 94

### [5-(2-{[5-(1H-Benzimidazole-2-ylamino)pentyl]amino}-2-oxoethyl)-2-oxo-2,3,4,6-tetrahydro-1H-1-benzazepine-1-yl]acetic acid

Preparation and purification analogously to example 92; ester stage ESI-MS [M+H⁺]=492. Chromatographic purification after cleavage of the ester afforded 20 mg as a white amorphous powder ESI-MS [M+H⁺]=478.

### Example 95

### {5-[2-({[4-(1H-Benzimidazole-2-ylamino)cyclohexyl]methyl}amino)-2-oxoethyl]-2-oxo-2,3,4,5-tetrahydro-1H-1-benzazepine-1-yl}acetic acid

Preparation and purification analogously to example 92; ester stage ESI-MS [M+H⁺]=518. Chromatographic purification after cleavage of the ester afforded 0.15 g as a white amorphous powder; ESI-MS [M+H⁺]=504.

### Example 96

### Ethyl-{1-[2-({[4-(1H-benzimidazole-2-ylamino)cyclohexyl]methyl}amino)-2-oxoethyl]-2-oxo-2,3,4,5-tetrahydro-1H-1-benzazepine-5-yl}acetate

0.6 g (2.4 mmol) N,N-Bis(2-oxo-3-oxazolidinyl)phosphoryl chloride were added to a solution of 0.8 g (1.6 mmol) of the acid of example XI, 0.15 g (3.2 mmol) ethyl alcohol, 0.2 g (1.6 mmol) 4-dimethylaminopyridine and 0.4 g (4 mmol) triethylamine in 60 ml CH₂Cl₂ and stirred overnight. After the reaction had completed 2 g glacial acetic acid and 3.7 g ethyl alcohol were added to the reaction solution and stirred for another 20 h at room temperature. For workup, 50 ml H₂O were added to the mixture, the organic phase washed with 10% K₂CO₃-solution and H₂O, dried over MgSO₄ and evaporated. Chromatographic purification of the residue (eluent: CH₂Cl₂/ethyl alcohol/50% glacial acetic acid 15/5/1) afforded 0.35 g of a white amorphous powder; ESI-MS [M+H⁺]=532.

Analogously to example 96 were prepared:

### Example 97

### Cyclohexyl-{1-[2-({[4-(1H-benzimidazole-2-ylamino)cyclohexyl]methyl}amino)-2-oxoethyl]-2-oxo-2,3,4,5-tetrahydro-1H-1-benzazepine-5-yl}acetate

0.26 g; ESI-MS [M+H⁺]=586.

### Example 98

### Neopentyl{1-[2-({[4-(1H-benzimidazole-2-ylamino)cyclohexyl]methyl}amino)-2-oxoethyl]-2-oxo-2,3,4,5-tetrahydro-1H-1-benzazepine-5-yl}acetate

0.21 g; ESI-MS [M+H⁺]=574.

### Example 99

### tert-Butyl [1-(2-{[4-(1H-benzimidazole-2-ylamino)benzyl]amino}-2-oxoethyl)-2-oxo-2,3,4,5-tetrahydro-1H-1-benzazepine-5-yl]acetate

Preparation was carried out analogously to example I by reaction of [5-(2-tert-butoxy-2-oxoethyl)-2-oxo-2,3,4,5-tetrahydro-1H-1-benzazepine-1-yl]acetic acid (3) with N-[4-(aminomethyl)phenyl]-1H-benzimidazole-2-amine (hydrochloride) (4). After chromatographic purification (eluent: CH₂Cl₂/ethyl alcohol/50% glacial acetic acid 45/5/0.3) the residue was dissolved in 70 ml CH₂Cl₂ and 5 ml CH₃OH, washed with 5% K₂CO₃-solution and H₂O, dried over Na₂SO₄ and evaporated to dryness. The amorphous residue was mixed with 25 ml CH₃OH in heat. 0.51 g of white crystals; Fp.: 231° C. (decomposition); ESI-MS [M+H⁺]=554.

### Example 100

### Cyclohexyl-[1-(2-{[4-(1H-benzimidazole-2-ylamino)benzyl]amino}-2-oxoethyl)-2-oxo-2,3,4,5-tetrahydro-1H-1-benzazepine-5-yl]acetate

0.64 g (1.28 mmol) of [1-(2-{[4-(1H-benzimidazole-2ylamino)benzyl]amino}-2-oxoethyl)-2-oxo-2,3,4,5-tetrahydro-1H-1-benzazepine-5-yl]acetic acid (acid of example III) was suspended in 30 ml cyclohexanol, 1.0 g HCl-gas introduced with stirring and the resulting yellow solution allowed to stand for 4 days at room temperature. Since an acid was still detectable by chromatography, it was heated for 7 h to 40-50° C. For workup, 100 ml diethylether were added, washed with K₂CO₃-solution and H₂O, dried over Na₂SO₄ and the solvent—finally for removal of cyclohexanol under oil pump vacuum and at a bath temperature of 50° C.—distilled off. The residue was purified chromatographically (eluent: CH₂Cl₂/acetone/methanol/50% acetic acid 45151410.3). 0.659 of a slightly beige coloured powder; ESI-MS [M+H⁺]=580.

### Example 101

### Ethyl-[1-(2-{[4-(1H-benzimidazole-2-ylamino)benzyl]amino}-2-oxoethyl)-2-oxo-2,3,4,6-tetrahydro-1H-1-benzazepine-6-yl]acetate

0.619 (1.23 mmol) of [1-(2-{[4-(1H-benzimidazole-2ylamino)benzyl]amino}-2-oxoethyl)-2-oxo-2,3,4,5-tetrahydro-1H-1-benzazepine-5-yl]acetic acid (acid of example III) was suspended in 30 ml ethyl alcohol, 0.4 g HCl-gas introduced and allowed to stand for 4 days at room temperature. The ethyl alcohol was distilled off in vacuum, the residue taken up in ethyl acetate, washed with 5% NaHCO₃- and NaCl-solution, dried over Na₂SO₄ and evaporated. After thick-layer chromatographic purification (eluent: CH₂Cl₂/ethyl alcohol/50% acetic acid 43/7/0.5) the eluate was diluted with some CH₂Cl₂ and for removal of the acetic acid washed with 50% NaHCO₃-solution. After drying over Na₂SO₄ it was evaporated and the residue converted to an amorphous powder filterable with suction by means of diethylether/n-hexane; 0.55 g; ESI-MS [M+H⁺]=526.

### Example 102

### 1-{[(Cyclohexyloxy)carbonyl]oxy}ethyl [1-(2-{[4-(1H-benzimidazole-2-ylamino)benzyl]amino}-2-oxoethyl)-2-oxo-2,3,4,5-tetrahydro-1H-1-benzazepine-5-yl]acetate

0.3 g K₂CO₃ were added at 3° C. to a solution of 0.6 g (1.2 mmol) of [1-(2-{[4-(1H-benzimidazole-2ylamino)benzyl]amino}-2-oxoethyl)-2-oxo-2,3,4,5-tetrahydro-1H-1-benzazepine-5-yl]acetic acid (add of example III) in 10 ml DMF with stirring, some crystals 18-crown-6, 0.4 g cyclohexyl-1-iodo ethylcarbonate (preparation from cyclohexyl-1-chloroethylcarbonate and NaI analogously to J. Antibiot. 1987, 40 (1), 81-90) dissolved in 5 ml CH₃CN were added dropwise and stirred for 20 min. After addition of 100 ml cold NaCl-solution it was extracted several times with ethyl acetate, the combined organic phases washed with NaCl-solution, dried over Na₂SO₄ and evaporated. The residue was purified by column chromatography (eluent: CH₂Cl₂/acetone/methanol/50% acetic acid 45/5/5/0.3), after evaporation of the solvent taken up in 50 ml CH₂Cl₂, washed with 5% NaHCO₃-solution, dried over Na₂SO₄ and again evaporated. 90 mg of a white amorphous powder; ESI-MS [M+H⁺]=668.

### Example 103

### [(5R)-1-(2-{[4-(1H-Benzimidazol-2-ylamino)benzyl]amino}-2-oxoethyl)-2-oxo-2,3,4,5-tetrahydro-1H-1-benzazepine-5-yl]acetic acid

1.0 g (3.0 mmol) of the left-rotating acid (building block 57) and 0.72 g (3.0 mmol) N-[4-(aminomethyl)phenyl]-1H-benzimidazole-2-amine (hydrochloride) (4) were reacted analogously to example I and the reaction product purified by column chromatography (eluent: CH₂Cl₂/acetone/methanol/50% acetic acid 45/5/4/0.3); ester stage ESI-MS [M+H⁺]=554. Cleavage of the tert-butyl ester with 4n HCl in dioxane afforded 0.82 g of a white amorphous powder; ESI-MS [M+H⁺]=498; [α]_{D}²⁰=−107.7° (K⁺-salt, c=1 in H₂O).

### Example 104

### [(5S)-1-(2-{[4-(1H-Benzimidazole-2-ylamino)benzyl]amino}-2-oxoethyl)-2-oxo-2,3,4,5-tetrahydro-1H-1-benzazepine-5-yl]acetic acid

Preparation analogously to example 103 starting from the dextrorotatory acid building block 58.

Ester stage: 1.5 g of a amorphous powder; ESI-MS [M+H⁺]=554.

Target product: 0.79 g of a white amorphous powder; ESI-MS [M+H⁺]=498.

### Example 105

### {(5R)-1-[2-({[4-(1H-Benzimidazole-2-ylamino)cyclohexyl]methyl}amino)-2-oxoethyl]-2-oxo-2,3,4,5-tetrahydro-1H-1-benzazepine-5-yl}acetic acid

Preparation analogously to example I from the left-rotating acid building block 57 and trans-N-{[4-(aminomethyl)cyclohexyl]}-1H-benzimidazole-2-amine (dihydrochloride) (11).

Ester stage: 0.9 g of a white amorphous powder; ESI-MS [M+H⁺]=560.

Target product: 0.67 g of a white amorphous powder; ESI-MS [M+H⁺]=504; [α]_{D}²⁰=−104° (K⁺-salt, c=1 in H₂O).

### Example 106

### {(SS)-1-[2-({[4-(1H-Benzimidazole-2-ylamino)cyclohexyl]methyl}amino)-2-oxoethyl]-2-oxo-2,3,4,5-tetrahydro-1H-1-benzazepine-5-yl}acetic acid

Preparation analogously to example 103 starting from the dextrorotatory acid building block 58.

Ester stage: 0.72 g of a amorphous powder; ESI-MS [M+H⁺]=560.

Target product: 0.56 g of a white amorphous powder; ESI-MS [M+H⁺]=504; [α]_{D}²⁰=+101.60 (K⁺-salt, c=1 in H₂O).

### Example 107

### [4-(2-{[4-(1H-Benzimidazole-2-ylamino)benzyl]amino}-2-oxoethyl)-5-oxo-5,6,7,8-tetrahydro-4H-thieno[3,2-b]azepine-8-yl]acetic acid

Analogously to example I 0.6 g (1.8 mmol) [8-(2-tert-butoxy-2-oxoethyl)-5-oxo-5,6,7,8-tetrahydro-4H-thieno[3,2-b]azepine-4-yl]acetic acid (61) were reacted with 0.42 g (1.8 mmol) N-[4-(aminomethyl)phenyl]-1H-benzimidazole-2-amine (hydrochloride) (4) and the reaction product purified by thick-layer chromatography (eluent: CH₂Cl₂/methanol/conc. NH₃ 451510.2); ESI-MS [M+H⁺]=560. Cleavage of the t-butyl group with 4n HCl in dioxane afforded 0.34 g of a slightly yellowish powder; ESI-MS [M+H⁺]=504.

### Example 108

### {4-[2-({[4-(1H-Benzimidazole-2-ylamino)cyclohexyl]methyl}amino)-2-oxoethyl]-5-oxo-5,6,7,8-tetrahydro-4H-thieno[3,2-b]azepine-8-yl}acetic acid

Analogously to example 107.

Ester stage: 100 mg of a white amorphous powder, ESI-MS [M+H⁺]=566.

Target product: 98 mg of a white amorphous powder; ESI-MS [M+H⁺]=510.

### II. BIOLOGICAL EXAMPLES

### Example 1

### Integrin αᵥβ₃ Assay

For the identification and assessment of integrin αᵥβ₃ ligands, a test system was used which was based on competition between the natural integrin αᵥβ₃ ligand vitronectin and the test substance for binding to solid phase-bound integrin αᵥβ₃.

### Procedure

Microtiter plates coated with 250 ng/ml of integrin αᵥβ₃ in 0.05 M NaHCO₃ pH 9.2; 0.1 ml/well;
saturation with 1% powdered milk/assay buffer; 0.3 ml/well; 0.5 h/RT
3× washing with 0.05% Tween 20/assay buffer
test substance in 0.1% powdered milk/assay buffer, 50 μl/well+0 μg/ml or 2 μg/ml of human vitronectin (Boehringer Ingelheim T007) in 0.1% powdered milk/assay buffer, 50 μl/well; 1 h/RT
3× washing with 0.05% Tween 20/assay buffer
1 μg/ml of anti human vitronectin antibody coupled to peroxidase (Kordia SAVN-APHRP) in 0.1% powdered milk/assay buffer; 0.1 ml/well; 1 h/RT
3× washing with 0.05% Tween 20/assay buffer
0.1 ml/well of peroxidase substrate
stop reaction with 0.1 ml/well of 2 M H₂SO₄
measurement of the absorption at 450 nm

Integrin αᵥβ₃: Human placenta is solubilized with Nonidet and integrin αᵥβ₃ affinity-purified on a GRGDSPK matrix (elution with EDTA). Impurities due to integrin α_{IIb}β₃ and human serum albumin, and the detergent and EDTA are removed by anion-exchange chromatography.

Assay buffer: 50 mM Tris pH 7.5; 100 mM NaCl; 1 mM CaCl₂; 1 mM MgCl₂; 10 μM MnCl₂

Peroxidase substrate: mix 0.1 ml of TMB solution (42 mM TMB in DMSO) and 10 ml of substrate buffer (0.1 M sodium acetate pH 4.9), then add 14.7 μl of 3% H₂O₂.

Various dilutions of the test substances are employed in the assay and the IC₅₀ values are determined (concentration of the ligand at which 50% of the ligand is displaced). The compound from Example VII showed the best result here.

### Example 2

### Integrin α_{IIb}β₃ Assay

The assay is based on competition between the natural integrin α_{IIb}β₃ ligand fibrinogen and the test substance for binding to integrin α_{IIb}β₃.

### Procedure

Coat microtiter plates with 10 μg/ml of fibrinogen (Calbiochem 341578) in 0.05 M NaHCO₃ pH 9.2; 0.1 ml/well;
saturate with 1% BSA/PBS; 0.3 ml/well; 30 min/RT
3× washing with 0.05% Tween 20/PBS
test substance in 0.1% BSA/PBS; 50 μl/well+200 μg/ml of integrin α_{IIb}β₃ (Kordia) in 0.1% BSA/PBS; 50 μl/well; 2 to 4 h/RT
3× washing as above
biotinylated anti-integrin α_{IIb}β₃ antibody (Dianova CBL 130 B); 1:1000 in 0.1% BSA/PBS; 0.1 ml/well; 2 to 4 h/RT.
3× washing as above
streptavidin-peroxidase complex (B.M. 1089153) 1:10000 in 0.1% BSA/PBS; 0.1 ml/well; 30 min/RT
3× washing as above
0.1 ml/well of peroxidase substrate
stop reaction using 0.1 ml/well of 2 M H₂SO₄
measurement of the absorption at 450 nm

Peroxidase substrate: mix 0.1 ml of TMB solution (42 mM TMB in DMSO) and 10 ml of substrate buffer (0.1 M Na acetate pH 4.9), then add 14.7 μl of 3% H₂O₂

Various dilutions of the test substances are employed in the assay and the IC₅₀ values are determined (concentration of the antagonist at which 50% of the ligand is displaced).

By comparison of the IC₅₀ values in the integrin α_{IIb}β₃ and integrin αᵥb₃ assay, the selectivity of the substances can be determined.

### Example 3

### CAM Assay

The CAM (chorioallantoic membrane) assay serves as a generally recognized model for the assessment of the in vivo activity of integrin αᵥβ₃ antagonists. It is based on the inhibition of angiogenesis and neovascularization of tumor tissue (Am. J. Pathol. 1975, 79, 597-618; Cancer Res. 1980, 40, 2300-2309; Nature 1987, 329, 630). The procedure is carried out analogously to the prior art. The growth of the chicken embryo blood vessels and of the transplanted tumor tissue can be readily monitored and assessed.

### Example 4

### Rabbit Eye Assay

In this in vivo model, the inhibition of angiogenesis and neovascularization in the presence of integrin αᵥβ₃ antagonists can be monitored and assessed analogously to Example 3. The model is generally recognized and is based on the growth of rabbit blood vessels starting from the edge in the corner of the eye (Proc. Natl. Acad. Sci. USA. 1994, 91, 4082-4085; Science 1976, 193, 70-72). The procedure is carried out analogously to the prior art.

We claim:

1. The use of compounds of the formula I
B-G-L I
as ligand of integrin receptors,
where B, G and L have the following meanings:
L is a structural element of the formula I_{L}
—U-T I_{L}
where
T is a group COOH, a radical hydrolyzable to COOH or a radical bioisosteric to COOH and
—U— is —(X_{L})ₐ—(CR_{L}¹R_{L}²)_{b}—, —CR_{L}¹═CR_{L}²—, ethynylene or ═CR_{L}¹—, where
a is 0 or 1,
b is 0, 1 or 2
X_{L} is CR_{L}³R_{L}⁴, NR_{L}⁵, oxygen or sulfur,
R_{L}¹, R_{L}², R_{L}³, R_{L}⁴
independently of one another are hydrogen, -T, —OH, —NR_{L}⁶R_{L}⁷, —CO—NH₂, a halogen radical, a branched or unbranched, optionally substituted C₁-C₆-alkyl, C₂-C₆-alkenyl, C₂-C₆-alkynyl, C₃-C₇-cycloalkyl, —CO—NH(C₁-C₆-alkyl), CO—N(C₁-C₆-alkyl)₂ or C₁-C₄-alkoxy radical, an optionally substituted radical C₁-C₂-alkylene-T, C₂-alkenylene-T or C₂-alkynylene-T, an optionally substituted aryl or arylalkyl radical or in each case independently of one another are two radicals R_{L}¹ and R_{L}² or R_{L}³ and R_{L}⁴, or optionally R_{L}¹ and R_{L}³, together are an optionally substituted 3- to 7-membered saturated or unsaturated carbocycle or heterocycle, which can contain up to three identical or different heteroatoms O, N, S,
R_{L}⁵, R_{L}⁶, R_{L}⁷
independently of one another are hydrogen, a branched or unbranched, optionally substituted C₁-C₆-alkyl, C₃-C₇-cycloalkyl, CO—O—C₁-C₆-alkyl, SO₂—C₁-C₆-alkyl or CO—C₁-C₆-alkyl radical or an optionally substituted CO—O-alkylenearyl, SO₂-aryl, CO-aryl, SO₂-alkylenearyl or CO-alkylenearyl radical,
G is a structural element of the formula I_{G} 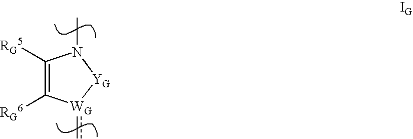
where
the structural element B is bonded to the structural element G via the ring nitrogen and the structural element L is bonded via W_{G},
Y_{G} is CO, CS, C═NR_{G}² or CR_{G}³R_{G}⁴,
R_{G}² is hydrogen, a hydroxyl group, a branched or unbranched, optionally substituted C₁-C₆-alkyl, C₁-C₄-alkoxy, C₃-C₇-cycloalkyl or —O—C₃-C₇-cycloalkyl radical or an optionally substituted aryl, —O-aryl, arylalkyl or —O-alkylenearyl radical,
R_{G}³, R_{G}⁴
independently of one another are hydrogen or a branched or unbranched, optionally substituted C₁-C₆-alkyl, C₂-C₆-alkenyl, C₂-C₆-alkynyl or C₁-C₄-alkoxy radical or both radicals R_{G}³ and R_{G}⁴ together a cyclic acetal —O—CH₂—CH₂—O— or —O—CH₂—O— or both radicals R_{G}³ and R_{G}⁴ together are an optionally substituted C₃-C₇-cycloalkyl radical,
R_{G}⁵ and R_{G}⁶
independently of one another are hydrogen, a hydroxyl group, a branched or unbranched, optionally substituted C₁-C₆-alkyl or C₁-C₄-alkoxy radical, an optionally substituted aryl or arylalkyl radical or both radicals R_{G}⁵ and R_{G}⁶ together are an optionally substituted, fused, unsaturated or aromatic 3- to 10-membered carbocycle or heterocycle, which can contain up to three different or identical heteroatoms O, N, S,
W_{G} is a structural element selected from the group of structural elements of the formulae I_{WG}¹ to I_{WG}⁴ 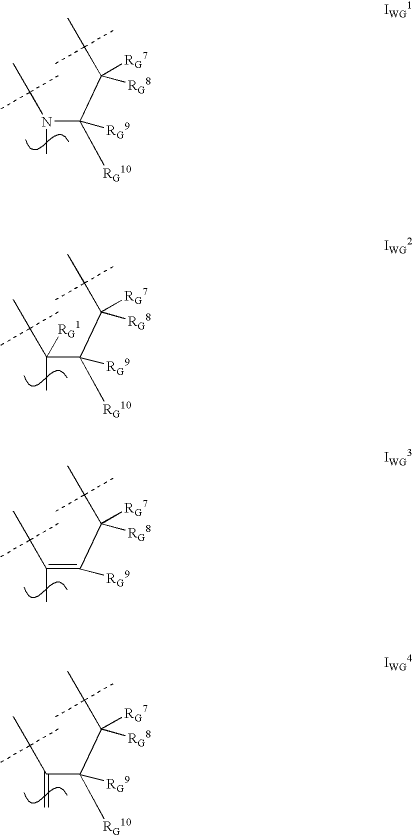
R_{G}¹ is hydrogen, halogen, a hydroxyl group or a branched or unbranched, optionally substituted C₁-C₆-alkyl or C₁-C₄-alkoxy radical,
R_{G}⁷, R_{G}⁸, R_{G}⁹, R_{G}¹⁰
independently of one another are hydrogen, a hydroxyl group, —CN, halogen, a branched or unbranched, optionally substituted C₁-C₆-alkyl, C₂-C₆-alkenyl, C₂-C₆-alkynyl, C₁-C₄-alkylene-C₃-C₇-cycloalkyl, C₁-C₄-alkylene-C₃-C₇-heterocycloalkyl or C₁-C₄-alkylene-C₃-C₇-heterocycloalkenyl radical, a branched or unbranched, optionally substituted radical C₁-C₄-alkylene-OR_{G}¹¹, C₁-C₄-alkylene-CO—OR_{G}¹¹, C₁-C₄-alkylene-O—CO—R_{G}¹¹, C₁-C₄-alkylene-CO—R_{G}¹¹, C₁-C₄-alkylene-SO₂—NR_{G}¹²R_{G}¹³, C₁-C₄-alkylene-CO—NR_{G}¹²R_{G}¹³, C₁-C₄-alkylene-O—CO—NR_{G}¹²R_{G}¹³, C₁-C₄-alkylene-NR_{G}¹²R_{G}¹³ or C₁-C₄-alkylene-SR_{G}¹¹, C₁-C₄-alkylene-SO—R_{G}¹¹, a radical —S—R_{G}¹¹, O—R_{G}¹¹, —SO—R_{G}¹¹, —SO₂—R_{G}¹¹, —CO—OR_{G}¹¹, —O—CO—R_{G}¹¹, —O—CO—NR_{G}¹²R_{G}¹³, —SO₂—NR_{G}¹²R_{G}¹³, —C1-NR_{G}¹²R_{G}¹³—NR_{G}¹²R_{G}¹³ or CO—R_{G}¹¹, an optionally substituted C₃-C₇-cycloalkyl, C₃-C₇-heterocycloalkyl, C₃-C₇-heterocycloalkenyl, aryl, hetaryl, arylalkyl or hetarylalkyl radical or in each case independently of one another two radicals R_{G}⁷ and R_{G}⁹ or R_{G}⁸ and R_{G}¹⁰ or R_{G}⁷ and R_{G}⁸ or R_{G}⁹ and R_{G}¹⁰ together are an optionally substituted, saturated or unsaturated, nonaromatic, 3- to 7-membered carbocycle or heterocycle which can contain up to 3 heteroatoms selected from the group O, N, S and up to two double bonds,
R_{G}¹¹ is hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- or bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical,
R_{G}¹², R_{G}¹³
independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- or bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹¹ and
R_{G}¹¹* is a radical R_{G}¹¹ which is independent of R_{G}¹¹
B is a structural element containing at least one atom which, under physiological conditions, as a hydrogen acceptor can form hydrogen bridges, where at least one hydrogen acceptor atom has a distance of 5 to 14 atomic bonds from structural element G along the shortest possible route along the structural element skeleton,
and the physiologically tolerable salts, prodrugs and the enantiomerically pure or diastereomerically pure and tautomeric forms.

2. The use as claimed in claim 1, wherein the structural element B is a structural element of the formula I_{B}
A-E- I_{B}
where A and E have the following meanings:
A is a structural element selected from the group:
a 4- to 8-membered monocyclic saturated, unsaturated or aromatic hydrocarbon which can contain up to 4 heteroatoms selected from the group O, N and S, where, in each case independently of one another, the optionally present ring nitrogen or the carbons can be substituted, with the proviso that at least one heteroatom selected from the group O, N and S is contained in the structural element A,
or
a 9- to 14-membered polycyclic, saturated, unsaturated or aromatic hydrocarbon which can contain up to 6 heteroatoms selected from the group N, O and S, where, in each case independently of one another, the ring nitrogen optionally contained or the carbon atoms can be substituted,
with the proviso that at least one heteroatom selected from the group O, N and S is contained in the structural element A,
a radical 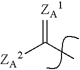
where
Z_{A}¹ is oxygen, sulfur or optionally substituted nitrogen and
Z_{A}² is optionally substituted nitrogen, oxygen or sulfur,
and a radical 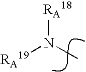
where
R_{A}¹⁸, R_{A}¹⁹
independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹¹,
and
E is a spacer structural element which covalently bonds the structural element A to the structural element G, where the number of atomic bonds along the shortest possible route along the structural element skeleton E is 5 to 14.

3. The use as claimed in one of claims 1 or 2, wherein the structural element A used is a structural element selected from the group of structural elements of the formulae I_{A}¹ to I_{A}¹⁸, 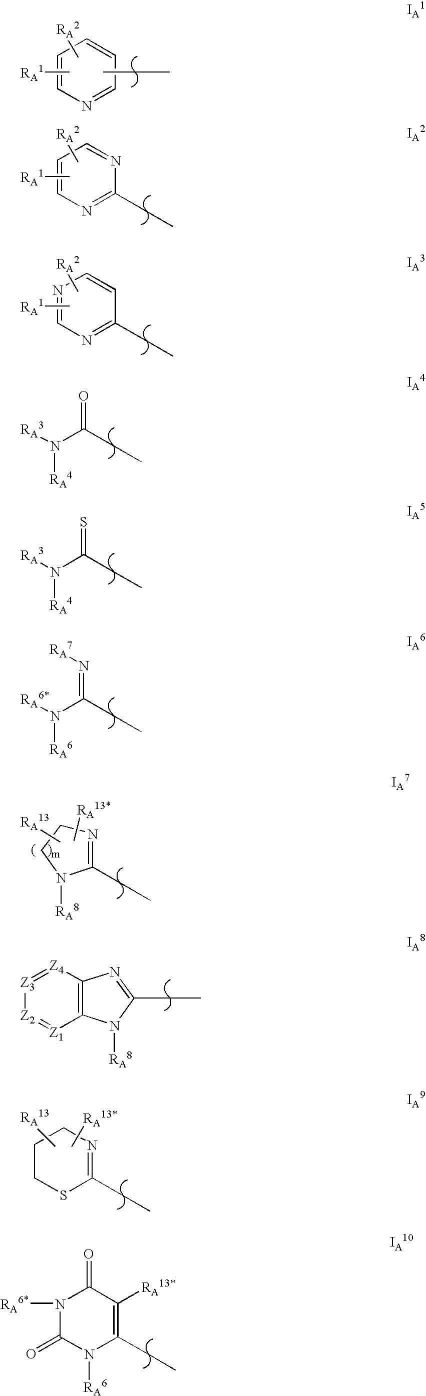 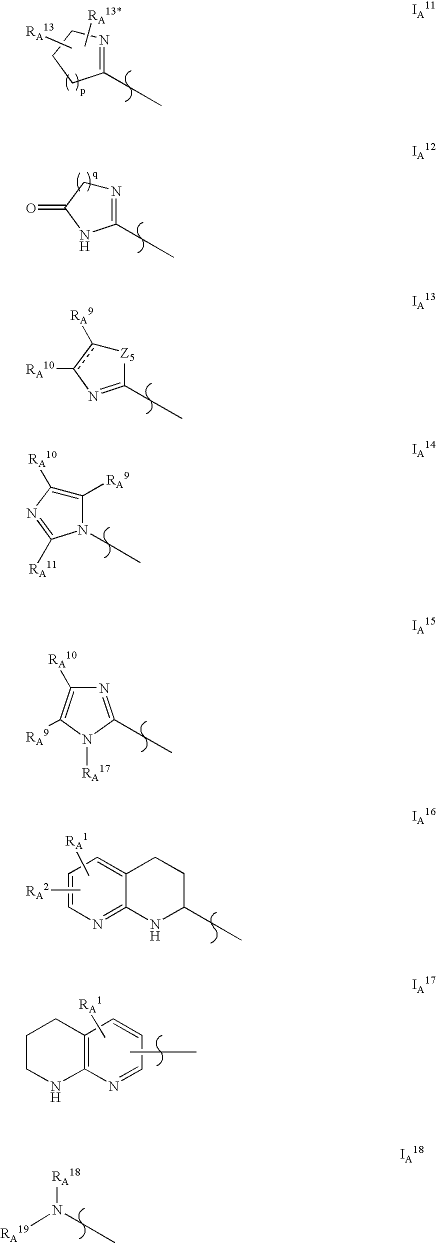
where
m, p, q
independently of one another are 1, 2 or 3,
R_{A}¹, R_{A}²
independently of one another are hydrogen, CN, halogen, a branched or unbranched, optionally substituted C₁-C₆-alkyl or CO—C₁-C₆-alkyl radical or an optionally substituted aryl, arylalkyl, hetaryl, hetarylalkyl or C₃-C₇-cycloalkyl radical or a radical CO—O—R_{A}¹⁴, O—R_{A}¹⁴, S—R_{A}¹⁴, NR_{A}¹⁵R_{A}¹⁶, CO—NR_{A}¹⁵R_{A}¹⁶ or SO₂NR_{A}¹⁵R_{A}¹⁶ or both radicals R_{A}¹ and R_{A}² together are a fused, optionally substituted, 5- or 6-membered, unsaturated or aromatic carbocycle or heterocycle which can contain up to three heteroatoms selected from the group O, N, and S,
R_{A}¹³, R_{A}¹³*
independently of one another are hydrogen, CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,
where
R_{A}¹⁴ is hydrogen, a branched or unbranched, optionally substituted C₁-C₆-alkyl, alkylene-C₁-C₄-alkoxy, C₂-C₆-alkenyl, C₂-C₆-alkynyl or C₁-C₆-alkylene-C₃-C₇-cycloalkyl radical or an optionally substituted C₃-C₇-cycloalkyl, aryl, arylalkyl, hetaryl or hetarylalkyl radical,
R_{A}¹⁵, R_{A}¹⁶,
independently of one another are hydrogen, a branched or unbranched, optionally substituted C₁-C₆-alkyl, CO—Cl₁-C₆-alkyl, SO₂—C₁-C₆-alkyl, COO—C₁-C₆-alkyl, CO—NH—C₁-C₆-alkyl, arylalkyl, COO-alkylenearyl, SO₂-alkylenearyl, CO—NH-alkylenearyl, CO—NH-alkylenehetaryl or hetarylalkyl radical or an optionally substituted C₃-C₇-cycloalkyl, aryl, CO-aryl, CO—NH-aryl, SO₂-aryl, hetaryl, CO—NH-hetaryl or CO-hetaryl radical,
R_{A}³, R_{A}⁴
independently of one another are hydrogen, —(CH₂)ₙ—(X_{A})ⱼ—R_{A}¹², or both radicals together are a 3- to 8-membered, saturated, unsaturated or aromatic N heterocycle which can additionally contain two further, identical or different heteroatoms O, N or S, where the cycle is optionally substituted or a further, optionally substituted, saturated, unsaturated or aromatic cycle can be fused to this cycle,
where
n is 0, 1, 2 or 3,
j is 0 or 1,
X_{A} is —CO—, —CO—N(R_{X}¹)—, —N(R_{X}¹)—CO—, —N(R_{X}¹)—CO—N(R_{X}¹*)—, —N(R_{X}¹)—CO—O—, —O—, —S—, —SO₂—, —SO₂—N(R_{X}¹)—, —SO₂—O—, —CO—O—, —O—CO—, —O—CO—N(R_{X}¹)—, —N(R_{X}¹)— or —N(R_{X}¹)—SO₂—,
R_{A}¹² is hydrogen, a branched or unbranched, optionally substituted C₁-C₆-alkyl radical, an optionally C₁-C₄-alkyl- or aryl-substituted C₂-C₆-alkynyl or C₂-C₆-alkenyl radical or a 3- to 6-membered, saturated or unsaturated heterocycle, substituted by up to three identical or different radicals, which can contain up to three different or identical heteroatoms O, N, S, a C₃-C₇-cycloalkyl, aryl or hetaryl radical, where two radicals together can be a fused, saturated, unsaturated or aromatic carbocycle or heterocycle which can contain up to three different or identical heteroatoms O, N, S, and the cycle can optionally be substituted or a further, optionally substituted, saturated, unsaturated or aromatic cycle can be fused to this cycle, or the radical R_{A}¹², together with R_{X}¹ or R_{X}¹* forms a saturated or unsaturated C₃-C₇-heterocycle which can optionally contain up to two further heteroatoms selected from the group O, S and N,
R_{X}¹, R_{X}¹*
independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl or SO₂—C₁-C₆-alkyl radical or an optionally substituted C₃-C₇-cycloalkyl, aryl, arylalkyl, CO—O-alkylenearyl, CO-alkylenearyl, CO-aryl, SO₂-aryl, hetaryl, CO-hetaryl or SO₂-alkylenearyl radical,
R_{A}⁶, R_{A}⁶*
are hydrogen, a branched or unbranched, optionally substituted C₁-C₄-alkyl, —CO—O—C₁-C₄-alkyl, arylalkyl, —CO—O-alkylenearyl, —CO—O-allyl, —CO—C₁-C₄-alkyl, —CO-alkylenearyl, C₃-C₇-cycloalkyl or —CO-allyl radical or in the structural element I_{A}⁷ both radicals R_{A}⁶ and R_{A}⁶* together are an optionally substituted, saturated, unsaturated or aromatic heterocycle which, in addition to the ring nitrogen, can contain up to two further different or identical heteroatoms O, N, S,
R_{A}⁷ is hydrogen, —OH, —CN, —CONH₂, a branched or unbranched, optionally substituted
C₁-C₄-alkyl, C₁-C₄-alkoxy, C₃-C₇-cycloalkyl or —O—CO—C₁-C₄-alkyl radical, or an optionally substituted arylalkyl, —O-alkylenearyl, —O—CO-aryl, —O—CO-alkylenearyl or —O—CO-allyl radical, or both radicals R_{A}⁶ and R_{A}⁷ together are an optionally substituted, unsaturated or aromatic heterocycle which, in addition to the ring nitrogen, can contain up to two further different or identical heteroatoms-O, N, S,
R_{A}⁸ is hydrogen, a branched or unbranched, optionally substituted C₁-C₄-alkyl, CO—C₁-C₄-alkyl, SO₂—C₁-C₄-alkyl or CO—O—C₁-C₄-alkyl radical or an optionally substituted aryl, CO-aryl, SO₂-aryl, CO—O-aryl, CO-alkylenearyl, SO₂-alkylenearyl, CO—O-alkylenearyl or alkylenearyl radical,
R_{A}⁹, R_{A}¹⁰
independently of one another are hydrogen, —CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶, or both: radicals R_{A}⁹ and R_{A}¹⁰ together in the structural element I_{A}¹⁴ are a 5- to 7-membered saturated, unsaturated or aromatic carbocycle or heterocycle which can contain up to three different or identical heteroatoms O, N, S and is optionally substituted by up to three identical or different radicals,
R_{A}¹¹ is hydrogen, —CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,
R_{A}¹⁷ is hydrogen or, in the structural element I_{A}¹⁶, both radicals R_{A}⁹ and R_{A}¹⁷ together are a 5- to 7-membered saturated, unsaturated or aromatic heterocycle which, in addition to the ring nitrogen, can contain up to three different or identical heteroatoms O, N, S and is optionally substituted by up to three identical or different radicals,
R_{A}¹, R_{A}¹⁹
independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- or bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—, —CO—OR_{G}¹¹, —CO—NR_{G}¹¹R_{G}¹¹* or —CO—R_{G}¹¹ which is independent of R_{G}¹¹
Z¹, Z², Z³, Z⁴
independently of one another are nitrogen, C—H, C-halogen or a branched or unbranched, optionally substituted C—C₁-C₄-alkyl or C—C₁-C₄-alkoxy radical,
Z⁵ is NR_{A}⁸, oxygen or sulfur.

4. The use as claimed in one of claims 1 to 3, wherein the spacer structural element E is composed of two to four substructural elements, selected from the group consisting of E¹ and E², where the sequence of linkage of the substructural elements is arbitrary and E¹ and E² have the following meanings:
E¹ is a substructural element of the formula I_{E1}
—(Y_{E})ₖ₁—(CR_{E}¹R_{E}²)_{c}-(Q_{E})ₖ₂-(CR_{E}³R_{E}⁴)_{d}— I_{E1}
and
E² is a substructural element of the formula I_{E2}
—(NR_{E}¹¹)ₖ₃—(CR_{E}⁵R_{E}⁶)_{f}-(Z_{E})ₖ₄-(CR_{E}⁹R_{E}¹⁰)_{g}—(X_{E})ₖ₅—(CR_{E}⁹R_{E}¹⁰)ₕ—(NR_{E}¹¹*)ₖ₆— I_{E2},
where
c, d, f, g, h
independently of one another are 0, 1 or 2,
k1, k2, k3, k4, k5, k6
independently of one another are 0 or 1,
X_{E}, Q_{E}
independently of one another are an optionally substituted 4- to 1-membered mono- or polycyclic, aliphatic or aromatic hydrocarbon which can contain up to 6 double bonds and up to 6 identical or different heteroatoms selected from the group N, O and S, where the ring carbons and/or the ring nitrogens can optionally be substituted,
Y_{E}, Z_{E}
independently of one another are CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴, C(═CR_{E}¹³R_{E}¹⁴), CR_{E}¹³═CR_{E}¹⁴, —CR_{E}¹³(OR_{E}¹⁵)—CHR_{E}¹⁴— or —CHR_{E}¹³—CR_{E}¹⁴(OR_{E}¹⁵)—,
R_{E}¹, R_{E}², R_{E}³, R_{E}⁴, R_{E}⁵, R_{E}⁶, R_{E}⁷, R_{E}⁸, R_{E}⁹, R_{E}¹⁰
independently of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)ₓ—(W_{E})_{z}—R_{E}¹⁷, an optionally substituted C₃-C₇-cycloalkyl, aryl, arylalkyl, hetaryl or hetarylalkyl radical, or
independently of one another in each case two radicals R_{E}¹ and R_{E}² or R_{E}³ and R_{E}⁴ or R_{E}⁵ and R_{E}⁶ or R_{E}⁷ and R_{E}⁸ or R_{E}⁹ and R_{E}¹⁰ together are a 3- to 7-membered, optionally substituted, saturated or unsaturated carbocycle or heterocycle which can contain up to three heteroatoms selected from the group O, N and S
x is 0, 1, 2, 3 or 4,
z is 0 or 1,
W_{E} is —CO—, —CO—N(R_{W}²)—, —N(R_{W}²)—CO—, —N(R_{W}²)—CO—N(R_{W}²*)—, —N(R_{W}²)—CO—O—, —O—, —S—, —SO₂—, —SO₂—N(R_{W}²)—, —SO₂—O—, —CO—O—, —O—CO—, —O—CO—N(R_{W}²)—, —N(R_{W}²)— or —N(R_{W}²)—SO₂—,
R_{W}², R_{W}²*
independently of one another are hydrogen, a branched or unbranched, optionally substituted C₁-C₆-alkyl, C₂-C₆-alkenyl, C₂-C₈-alkynyl, CO—C₁-C₆-alkyl CO—O—C₁-C₆-alkyl or SO₂—C₁-C₆-alkyl radical or an optionally substituted hetaryl, hetarylalkyl, arylalkyl, C₃-C₇-cycloalkyl, CO—O-alkylenearyl, CO-alkylenearyl, CO-aryl, SO₂-aryl, CO-hetaryl or SO₂-alkylenearyl radical,
R_{E}¹⁷ is hydrogen, a hydroxyl group, CN, halogen, a branched or unbranched, optionally substituted C₁-C₆-alkyl radical, an optionally substituted C₃-C₇-cycloalkyl, aryl, hetaryl or arylalkyl radical, a C₂-C₆-alkynyl or C₂-C₆-alkenyl radical optionally substituted by C₁-C₄-alkyl or aryl, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocycle substituted by up to three identical or different radicals, which can contain up to three different or identical heteroatoms O, N, S, where two radicals can together be a fused, saturated, unsaturated or aromatic carbocycle or heterocycle which can contain up to three different or identical heteroatoms O, N, S and the cycle can optionally be substituted or a further, optionally substituted, saturated, unsaturated or aromatic cycle can be fused to this cycle, or the radical R_{E}¹⁷ forms, together with R_{W}² or R_{W}²*, a saturated or unsaturated C₃-C₇-heterocycle which can optionally contain up to two further heteroatoms selected from the group O, S and N,
R_{E}¹¹, R_{E}¹¹*
independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl, CO—NHC₁-C₆-alkoxyalkyl, CO—NHC₁-C₆-alkyl or SO₂—C₁-C₆-alkyl radical or an optionally substituted hetaryl, arylalkyl, C₃-C₇-cycloalkyl, CO—O-alkylenearyl, CO—NH-alkylenearyl, CO-alkylenearyl, CO-aryl, CO—NH-aryl, SO₂-aryl, CO-hetaryl, SO₂-alkylenearyl, SO₂-hetaryl or SO₂-alkylenehetaryl radical,
R_{E}¹² is hydrogen, a branched or unbranched, optionally substituted C₁-C₆-alkyl, C₂-C₆-alkenyl, C₂-C₈-alkynyl radical, an optionally substituted C₃-C₇-cycloalkyl, hetaryl, arylalkyl or hetarylalkyl radical or a radical CO—R_{E}¹⁶, COOR_{E}¹⁶ or SO₂—R_{E}¹⁶,
R_{E}¹³, R_{E}¹⁴
independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁵ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁶ is hydrogen, a hydroxyl group, a branched or unbranched, optionally substituted C₁-C₆-alkyl, C₂-C₆-alkenyl, C₂-C₆-alkynyl or C₁-C₅-alkylene-C₁-C₄-alkoxy radical, or an, optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical.

5. The use as claimed in one of claims 1 to 4, wherein the spacer structural-element E used is a structural element of the formula I_{E1E2}
-E₂-E₁- I_{E1E2}
and E¹ and E² have the following meanings:
E¹ is a substructural element of the formula I_{E1}
—(Y_{E})ₖ₁—(CR_{E}¹R_{E}²)_{c}-(Q_{E})ₖ₂-(CR_{E}³R_{E}⁴)_{d}— I_{E1}
and
E² is a substructural element of the formula I_{E2}
—(NR_{E}¹¹)ₖ₃—(CR_{E}⁵R_{E}⁶)_{f}-(Z_{E})ₖ₄-(CR_{E}⁷R_{E}⁸)_{g}—(X_{E})ₖ₅—(CR_{E}⁹R_{E}¹⁰)ₕ—(NR_{E}¹¹*)ₖ₆—I_{E2},
where
c, d, f, g, h
independently of one another are 0, 1 or 2,
k1, k2, k3, k4, k5, k6
independently of one another are 0 or 1,
X_{E}, Q_{E}
independently of one another are an optionally substituted 4- to 11-membered mono- or polycyclic, aliphatic or aromatic hydrocarbon which can contain up to 6 double bonds and up to 6 identical or different heteroatoms selected from the group N, O and S, where the ring carbons and/or the ring nitrogens can optionally be substituted,
Y_{E}, Z_{E}
independently of one another are CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴, C(═CR_{E}¹³R_{E}¹⁴), CR_{E}¹³═CR_{E}¹⁴, —CR_{E}¹³(OR_{E}¹⁵)—CHR_{E}¹⁴— or —CHR_{E}¹³—CR_{E}¹⁴ (OR_{E}¹⁵)—,
R_{E}¹, R_{E}², R_{E}³, R_{E}⁴, R_{E}⁵, R_{E}⁶, R_{E}⁷, R_{E}⁸, R_{E}⁹, R_{E}¹⁰
independently of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)ₓ—(W_{E})_{z}—R_{E}¹⁷, an optionally substituted C₃-C₇-cycloalkyl, aryl, arylalkyl, hetaryl or hetarylalkyl radical or independently of one another are in each case two radicals R_{E}¹ and R_{E}² or R_{E}³ and R_{E}⁴ or R_{E}⁵ and R_{E}⁶ or R_{E}⁷ and R_{E}⁸ or R_{E}⁹ and R_{E}¹⁰ together are a 3- to 7-membered, optionally substituted, saturated or unsaturated carbo- or heterocycle, which can contain up to three heteroatoms selected from the group O, N and S,
x is 0, 1, 2, 3 or 4,
z is 0 or 1,
W_{E} is —CO—, —CO—N(R_{W}²)—, —N(R_{W}²)—CO—, N(R_{W}²)—CO—N(R_{W}²*)—, —N(R_{W}²)—CO—O—, —O—, —S—, —SO₂—, —SO₂—N(R_{W}²)—, —SO₂—O—, —CO—O—, —O—CO—, —O—CO—N(R_{W}²)—, —N(R_{W}²)— or —N(R_{W}²)—SO₂—,
R_{W}², R_{W}²*
independently of one another are hydrogen, a branched or unbranched, optionally substituted C₁-C₆-alkyl, C₂-C₆-alkenyl, C₂-C₈-alkynyl, CO—C₁-C₆-alkyl, CO—O—C₁-C₆-alkyl or SO₂—C₁-C₆-alkyl radical or an optionally substituted hetaryl, hetarylalkyl, arylalkyl, C₃-C₇-cycloalkyl, CO—O-alkylenearyl, CO-alkylenearyl, CO-aryl, SO₂-aryl, CO-hetaryl or SO₂-alkylenearyl radical,
R_{E}¹⁷ is hydrogen, a hydroxyl group, CN, halogen, a branched or unbranched, optionally substituted C₁-C₆-alkyl radical, an optionally substituted C₃-C₇-cycloalkyl, aryl, hetaryl or arylalkyl radical, a C₂-C₆-alkynyl or C₂-C₆-alkenyl radical optionally substituted by C₁-C₄-alkyl or aryl, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocycle substituted by up to three identical or different radicals, which can contain up to three different or identical heteroatoms O, N, S where two radicals together can be a fused, saturated, unsaturated or aromatic carbocycle or heterocycle which can contain up to three different or identical heteroatoms O, N, S and the cycle can optionally be substituted or a further, optionally substituted, saturated, unsaturated or aromatic cycle can be fused to this cycle, or the radical R_{E}¹⁷ forms together with R_{W}² or R_{W}²* a saturated or unsaturated C₃-C₇-heterocycle, which can optionally contain up to two further heteroatoms selected from the group O, S and N,
R_{E}¹¹, R_{E}¹¹*
independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl, CO—NH—C₁-C₆-alkoxyalkyl, CO—NH—C₁-C₆-alkyl or SO₂—C₁-C₆-alkyl radical or an optionally substituted hetaryl, arylalkyl, C₃-C₇-cycloalkyl, CO—O-alkylenearyl, CO—NH-alkylenearyl, CO-alkylenearyl, CO-aryl, CO—NH-aryl, SO₂-aryl, CO-hetaryl, SO₂-alkylenearyl, SO₂-hetaryl or SO₂-alkylenehetaryl radical,
R_{E}¹² is hydrogen, a branched or unbranched, optionally substituted C₁-C₆-alkyl, C₂-C₆-alkenyl, C₂-C₈-alkynyl, an optionally substituted C₃-C₇-cycloalkyl, hetaryl, arylalkyl or hetarylalkyl radical or a radical CO—R_{E}¹⁶, COOR_{E}¹⁶ or SO₂—R_{E}¹⁶,
R_{E}¹³, R_{E}¹⁴
independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁵ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁶ is hydrogen, a hydroxyl group, a branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical.

6. The use of the structural element of the formula I_{GL}
-G-L, I_{GL}
for the preparation of compounds which bind to integrin receptors,
where G and L have the following meanings:
L is a structural element of the formula I_{L}
—U-T I_{L}
where
T is a group COOH, a radical hydrolyzable to COOH or a radical bioisosteric to COOH and
—U— is —(X_{L})ₐ—(CR_{L}¹R_{L}²)_{b}—, —CR_{L}¹═CR_{L}²—, ethynylene or ═CR_{L}¹—, where
a is 0 or 1,
b is 0, 1 or 2
X_{L} is CR_{L}³R_{L}⁴, NR_{L}⁵, oxygen or sulfur,
R_{L}¹, R_{L}², R_{L}³, R_{L}⁴
independently of one another are hydrogen, -T, —OH, —NR_{L}⁶R_{L}⁷, —CO—NH₂, a halogen radical, a branched or unbranched, optionally substituted C₁-C₆-alkyl, C₂-C₆-alkenyl, C₂-C₆-alkynyl, C₃-C₇-cycloalkyl, —CO—NH(C₁-C₆-alkyl), —CO—N(C₁-C₆-alkyl)₂ or C₁-C₄-alkoxy radical, an optionally substituted radical C₁-C₂-alkylene-T, C₂-alkenylene-T or C₂-alkynylene-T, an optionally substituted aryl or arylalkyl radical or in each case independently of one another are two radicals R_{L}¹ and R_{L}² or R_{L}³ and R_{L}⁴, or optionally R_{L}¹ und R_{L}³ together are an optionally substituted 3- to 7-membered saturated or unsaturated carbocycle or heterocycle which can contain up to three different or identical heteroatoms O, N, S,
R_{L}⁵, R_{L}⁶, R_{L}⁷
independently of one another are hydrogen, a branched or unbranched, optionally substituted C₁-C₆-alkyl, C₃-C₇-cycloalkyl, CO—O—C₁-C₆-alkyl, SO₂—C₁-C₆-alkyl or CO—C₁-C₆-alkyl radical or an optionally substituted CO—O-alkylenearyl, SO₂-aryl, CO-aryl, SO₂-alkylenearyl or CO-alkylenearyl radical,
G is a structural element of the formula I_{G} 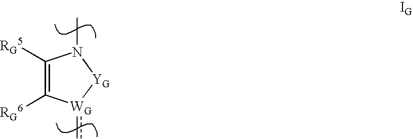
where
the structural element B is bonded to the structural element G via the ring nitrogen and the structural element L is bonded via W_{G},
Y_{G} is CO, CS, C═NR_{G}² or CR_{G}³R_{G}⁴,
R_{G}² is hydrogen, a hydroxyl group, a branched or unbranched, optionally substituted C₁-C₆-alkyl, C₁-C₄-alkoxy, C₃-C₇-cycloalkyl or —O—C₃-C₇-cycloalkyl radical or an optionally substituted aryl, —O-aryl, arylalkyl or —O-alkylenearyl radical,
R_{G}³, R_{G}⁴
independently of one another are hydrogen or a branched or unbranched, optionally substituted C₁-C₆-alkyl, C₂-C₆-alkenyl, C₂-C₆-alkynyl or C₁-C₄-alkoxy radical or both radicals R_{G}³ and R_{G}⁴ together are a cyclic acetal —O—CH₂—CH₂—O— or —O—CH₂—O— or both radicals R_{G}³ and R_{G}⁴ together are an optionally substituted, C₃-C₇-cycloalkyl radical,
R_{G}⁵ and R_{G}⁶
independently of one another are hydrogen, a hydroxyl group, a branched or unbranched, optionally substituted C₁-C₆-alkyl or C₁-C₄-alkoxy radical, an optionally substituted aryl or arylalkyl radical or both radicals R_{G}⁵ and R_{G}⁶ together are an optionally substituted, fused, unsaturated or aromatic 3- to 10-membered carbocycle or heterocycle, which can contain up to three different or identical heteroatoms O, N, S,
with the proviso that in the case of this fused, unsaturated or aromatic 3- to 6-membered carbocycle or heterocycle substituents are excluded which contain a structural element —V—CO—R⁸, where
V is an optionally substituted C₁-C₂-alkylene radical
R⁸ is a hydroxyl group, a C₁-C₈-alkoxy, aryl-C₀-C₆-alkoxy, C₁-C₈-alkylcarbonyloxy-C₁-C₄-alkoxy or aryl-C₁-C₈-alkylcarbonyloxy-C₁-C₄-alkoxy group or an L- or D-amino acid, which is bonded by an amide bond and in which the carboxylic acid component of said amino acid is present as a free acid or esterified with C₁-C₆-alkyl,
W_{G} is a structural element selected from the group of structural elements of the formulae I_{WG}¹ to I_{WG}⁴, 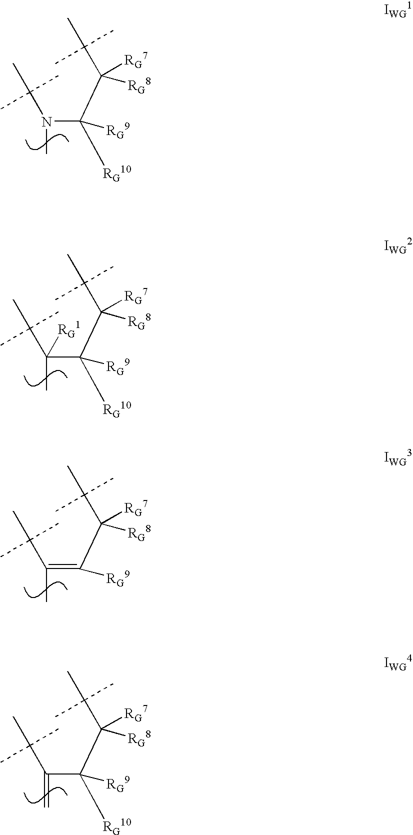
R_{G}¹ is hydrogen, halogen, a hydroxyl group or a branched or unbranched, optionally substituted C₁-C₆-alkyl or C₁-C₄-alkoxy radical,
R_{G}⁷, R_{G}⁸, R_{G}⁹, R_{G}¹⁰
independently of one another are hydrogen, a hydroxyl group, —CN, halogen, a branched or unbranched, optionally substituted C₁-C₆-alkyl, C₂-C₆-alkenyl, C₂-C₆-alkynyl, C₁-C₄-alkylene-C₃-C₇-cycloalkyl, C₁-C₄-alkylene-C₃-C₇-heterocycloalkyl or C₁-C₄-alkylene-C₃-C₇-heterocycloalkenyl radical, a branched or unbranched, optionally substituted radical C₁-C₄-alkylene-OR_{G}¹¹, C₁-C₄-alkylene-CO—OR_{G}¹¹, C₁-C₄-alkylene-O—CO—R_{G}¹¹, C₁-C₄-alkylene-CO—R_{G}¹¹, C₁-C₄-alkylene-SO₂—NR_{G}¹²R_{G}¹³, C₁-C₄-alkylene-CO—NR_{G}¹²R_{G}¹³, C₁-C₄-alkylene-O—CO—NR_{G}¹²R_{G}¹³, C₁-C₄-alkylene-NR_{G}¹²R_{G}¹³ or C₁-C₄-alkylene-SR_{G}¹¹, C₁-C₄-alkylene-SO—R_{G}¹¹, a radical —S—R_{G}¹¹, —O—R_{G}¹¹, —SO—R_{G}¹¹, —SO₂—R_{G}¹¹, —CO—OR_{G}¹¹, —O—CO—R_{G}¹¹, —O—CO—NR_{G}¹²R_{G}¹³, —SO₂—NR_{G}¹²R_{G}¹³, —CO—N¹²R_{G}¹³, —NR_{G}¹²R_{G}¹³ or CO—R_{G}¹¹, an optionally substituted C₃-C₇-cycloalkyl, C₃-C₇-heterocycloalkyl, C₃-C₇-heterocycloalkenyl, aryl, hetaryl, arylalkyl or hetarylalkyl radical or in each case independently of one another two radicals R_{G}⁷ and R_{G}⁹ or R_{G}⁸ and R_{G}¹⁰ or R_{G}⁷ and R_{G}⁸ or R_{G}⁹ and R_{G}¹⁰ together are an optionally substituted, saturated or unsaturated, nonaromatic, 3- to 7-membered carbocycle or heterocycle which can contain up to 3 heteroatoms selected from the group O, N, S and which can contain up to two double bonds,
R_{G}¹¹ is hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical,
R_{G}¹², R_{G}¹³
independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹¹ and
R_{G}¹¹* is a radical R_{G}¹¹ which is independent of R_{G}¹¹.

7. The use of the compounds as claimed in one of claims 1 to 5 as ligands of the α_{V}β₃ integrin receptor.

8. The use of the compounds as claimed in one of claims 1 to 5 for the production of drugs for treating diseases in which the interaction between integrins and their natural ligands is excessive or decreased.

9. The use of the compounds as claimed in one of claims 1 to 5 as claimed in claim 8 for the treatment of diseases in which the interaction between α_{V}β₃ ingegrin and its natural ligands is excessive or decreased.

10. The use of the compounds as claimed in one of claims 1 to 5 as claimed in claim 9 for the treatment of atherosclerosis, rheumatoid arthritis, restenosis after vascular injury or stent implantation, angioplasty, acute kidney failure, angiogenesis-associated microangiopathies, diabetic angiopathies, blood platelet-mediated vascular occlusion, arterial thrombosis, congestive heart failure, myocardial infarct, stroke, cancer, osteoporosis, high blood pressure, psoriasis or viral, parasitic or bacterial conditions, inflammation, wound healing, hyperparathyroidism, Paget's disease, malignant hypercalcemia or metastatic osteolytic lesions.

11. A compound of the formula I′
A-E′-G′-L I′
where A, E′, G′ and L have the following meanings:
L is a structural element of the formula I_{L}
—U-T I_{L}
where
T is a group COOH, a radical hydrolyzable to COOH or a radical bioisosteric to COOH and
—U— is —(X_{L})ₐ—(CR_{L}¹R_{L}²)_{b}—, —CR_{L}¹═CR_{L}²—, ethynylene or ═CR_{L}¹—, where
a is 0 or 1,
b is 0, 1 or 2
X_{L} is CR_{L}³R_{L}⁴, NR_{L}⁵, oxygen or sulfur,
R_{L}¹, R_{L}², R_{L}³, R_{L}⁴
independently of one another are hydrogen, -T, —OH, —NR_{L}⁶R_{L}⁷, —CO—NH₂, a halogen radical, a branched or unbranched, optionally substituted C₁-C₆-alkyl, C₂-C₆-alkenyl, C₂-C₆-alkynyl, C₃-C₇-cycloalkyl, —CO—NH(C₁-C₆-alkyl), —CO—N(C₁-C₆-alkyl)₂ or C₁-C₄-alkoxy radical, an optionally substituted radical C₁-C₂-alkylene-T, C₂-alkenylene-T or C2-alkynylene-T, an optionally substituted aryl or arylalkyl radical or in each case independently of one another are two radicals R_{L}¹ and R_{L}² or R_{L}³ and R_{L}⁴, or optionally R_{L}¹ and R_{L}³ together are an optionally substituted 3- to 7-membered saturated or unsaturated carbocycle or heterocycle, which can contain up to three identical or different heteroatoms O, N, S,
R_{L}⁵, R_{L}⁶, R_{L}⁷
independently of one another are hydrogen, a branched or unbranched, optionally substituted C₁-C₆-alkyl, C₃-C₇-cycloalkyl, CO—O—C₁-C₆-alkyl, SO₂—C₁-C₆-alkyl or CO—C₁-C₆-alkyl radical or an optionally substituted CO—O-alkylenearyl, SO₂-aryl, CO-aryl, SO₂-alkylenearyl or CO-alkylenearyl radical,
G′ is a structural element of the formula I_{G} 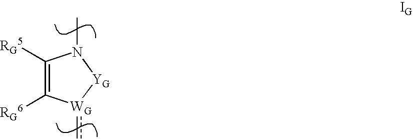
where
the structural element A-E′ is bonded to the structural element G′ via the ring nitrogen and the structural element L is bonded via W_{G},
Y_{G} is CO, CS, C═NR_{G}² or CR_{G}³R_{G}⁴,
R_{G}² is hydrogen, a hydroxyl group, a branched or unbranched, optionally substituted C₁-C₆-alkyl, C₁-C₄-alkoxy, C₃-C₇-cycloalkyl or —O—C₃-C₇-cycloalkyl radical or an optionally substituted aryl, —O-aryl, arylalkyl or —O-alkylenearyl radical,
R_{G}³, R_{G}⁴
independently of one another are hydrogen or a branched or unbranched, optionally substituted C₁-C₆-alkyl, C₂-C₆-alkenyl, C₂-C₆-alkynyl or C₁-C₄-alkoxy radical or both radicals R_{G}³ and R_{G}⁴ together are a cyclic acetal —O—CH₂—CH₂—O— or —O—CH₂—O— or both radicals R_{G}³ and R_{G}⁴ together are an optionally substituted C₃-C₇-cycloalkyl radical,
R_{G}⁵ and R_{G}⁶
independently of one another are hydrogen, a hydroxyl group, a branched or unbranched, optionally substituted C₁-C₆-alkyl or C₁-C₄-alkoxy radical, an optionally substituted aryl or arylalkyl radical or both radicals R_{G}⁵ and R_{G}⁶ together are an optionally substituted, fused, unsaturated or aromatic 3- to 10-membered carbocycle or heterocycle, which can contain up to three different or identical heteroatoms O, N, S,
W_{G} is a structural element selected from the group of structural elements of the formulae I_{WG}¹ to I_{WG}⁴, 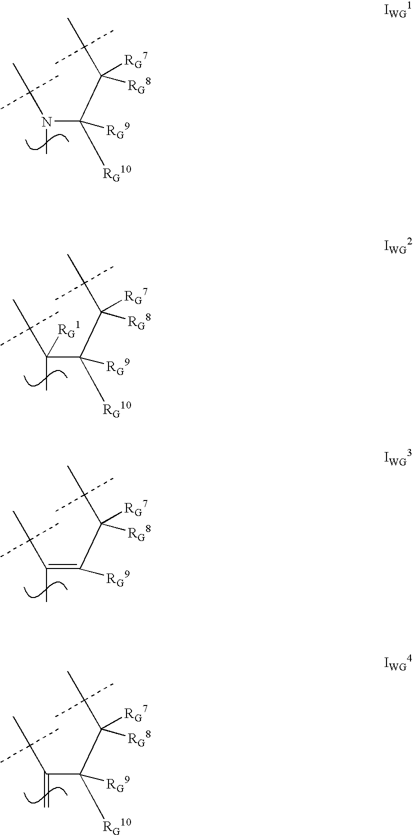
R_{G}¹ is hydrogen, halogen, a hydroxyl group or a branched or unbranched, optionally substituted C₁-C₆-alkyl or C₁-C₄-alkoxy radical,
R_{G}⁷, R_{G}⁸, R_{G}⁹, R_{G}¹⁰
independently of one another are hydrogen, a hydroxyl group, —CN, halogen, a branched or unbranched, optionally substituted C₁-C₆-alkyl, C₂-C₆-alkenyl, C₂-C₆-alkynyl, C₁-C₄-alkylene-C₃-C₇-cycloalkyl, C₁-C₄-alkylene-C₃-C₇-heterocycloalkyl or C₁-C₄-alkylene-C₃-C₇-heterocycloalkenyl radical, a branched or unbranched, optionally substituted radical C₁-C₄-alkylene-OR_{G}¹¹, C₁-C₄-alkylene-CO—OR_{G}¹¹, C₁-C₄-alkylene-O—CO—R_{G}¹¹, C₁-C₄-alkylene-CO—R_{G}¹¹, C₁-C₄-alkylene-SO₂—NR_{G′}¹²R_{G′}¹³, C₁-C₄-alkylene-CO—NR_{G′}¹²R_{G′}¹³, C₁-C₄-alkylene-O—CO—NR_{G′}¹²R_{G′}¹³, C₁-C₄-alkylene-NR_{G′}¹²R_{G′}¹³ or C₁-C₄-alkylene-SR_{G}¹¹, C₁-C₄-alkylene-SO—R_{G}¹¹, a radical —S—R_{G}¹¹, —O—R_{G}¹¹, —SO—R_{G}¹¹, —SO₂—R_{G}¹¹, —CO—OR_{G}¹¹, —O—CO—R_{G}¹¹, —O—CO—NR_{G′}¹²R_{G′}¹³, —SO₂—NR_{G′}¹²R_{G′}¹³, —CO—NR_{G′}¹²R_{G′}¹³, —NR_{G′}¹²R_{G′}¹³ or CO—R_{G}¹¹, an optionally substituted C₃-C₇-cycloalkyl, C₃-C₇-heterocycloalkyl, C₃-C₇-heterocycloalkenyl, aryl, hetaryl, arylalkyl or hetarylalkyl radical or in each case independently of one another two radicals R_{G}⁷ and R_{G}⁹ or R_{G}⁸ and R_{G}¹⁰ or R_{G}⁷ and R_{G}⁸ or R_{G}⁹ and R_{G}¹⁰ together are an optionally substituted, saturated or unsaturated, nonaromatic, 3- to 7-membered carbocycle or heterocycle which can contain up to 3 heteroatoms selected from the group O, N, S and up to two double bonds,
R_{G}¹¹ is hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical,
R_{G′}¹², R_{G′}¹³
independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹¹, and
R_{G}¹¹*
is a radical R_{G}¹¹ which is independent of R_{G}¹¹,
R_{G}¹⁴ is hydrogen, a branched or unbranched, optionally substituted C₁-C₈-alkyl, C₂-C₆-alkenyl, C₂-C₆-alkynyl or C₁-C₅-alkylene-C₁-C₄-alkoxy radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-heterocycloalkyl, C₁-C₄-alkylene-heterocycloalkenyl or hetarylalkyl radical,
E′ is a structural element, composed of two to four substructural elements, selected from the group E¹ and E², where the sequence of linkage of the substructural elements is arbitrary and E¹ and E² have the following meanings:
E¹ is a substructural element of the formula I_{E1}
—(Y_{E})ₖ₁—(CR_{E}¹R_{E}²)_{c}-(Q_{E})ₖ₂-(CR_{E}³R_{E}⁴)_{d}— I_{E1}
and
E² is a substructural element of the formula I_{E2}
—(NR_{E}¹¹)ₖ₃—(CR_{E}⁵R_{E}⁶)_{f}-(Z_{E})ₖ₄-(CR_{E}⁷R_{E}⁸)_{g}—(X_{E})ₖ₅—(CR_{E}⁹R_{E}¹⁰)ₕ—(NR_{E}¹¹*)ₖ₆— I_{E2},
where
c, d, f, g, h
independently of one another are 0, 1 or 2,
k1, k2, k3, k4, k5, k6
independently of one another are 0 or 1,
X_{E}, Q_{E}
independently of one another are an optionally substituted 4- to 11-membered mono- or polycyclic, aliphatic or aromatic hydrocarbon which can contain up to 6 double bonds and up to 6 identical or different heteroatoms selected from the group N, O and S, where the ring carbons and/or the ring nitrogens can optionally be substituted,
Y_{E}, Z_{E}
independently of one another are CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO-o, O—CO, oxygen, ethynylene, CR_{E}¹³—O—CR_{E}¹⁴, C(═CR_{E}¹³R_{E}¹⁴), CR_{E}¹³═CR_{E}¹⁴, —CR_{E}¹³(OR_{E}¹⁵)—CHR_{E}¹⁴— or —CHR_{E}¹³—CR_{E}¹⁴(OR_{E}¹⁵)—,
R_{E}¹, R_{E}², R_{E}³, R_{E}⁴, R_{E}⁵, R_{E}⁶, R_{E}⁷, R_{E}⁸, R_{E}⁹, R_{E}¹⁰
independently of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)ₓ—(W_{E})_{z}—R_{E}¹⁷, an optionally substituted C₃-C₇-cycloalkyl, aryl, arylalkyl, hetaryl or hetarylalkyl radical, or
independently of one another in each case two radicals R_{E}¹ and R_{E}² or R_{E}³ and R_{E}⁴ or R_{E}⁸ and R_{E}⁶ or R_{E}⁷ and R_{E}⁸ or R_{E}⁹ and R_{E}¹⁰ together are a 3- to 7-membered, optionally substituted, saturated or unsaturated carbocycle or heterocycle which can contain up to three heteroatoms selected from the group O, N and S,
x is 0, 1, 2, 3 or 4,
z is 0 or 1,
W_{E} is —CO—, —CO—N(R_{W}²)—, —N(R_{W}²)—CO—, —N(R_{W}²)—C—N(R_{W}²*)—, —N(R_{W}²)—CO—O—, —O—, —S—, —SO₂—, —SO₂—N(R_{W}²)—, —SO₂—O—, —CO—O—, —O—CO—, —O—CO—N(R_{W}²)—, —N(R_{W}¹²)— or —N(R_{W}²)—SO₂—,
R_{W}², R_{W}²*
independently of one another are hydrogen, a branched or unbranched, optionally substituted C₁-C₆-alkyl, C₂-C₆-alkenyl, C₂-C₈-alkynyl, CO—C₁-C₆-alkyl CO—O—C₁-C₆-alkyl or SO₂—C₁-C₆-alkyl radical or an optionally substituted hetaryl, hetarylalkyl, arylalkyl, C₃-C₇-cycloalkyl, CO—O-alkylenearyl, CO-alkylenearyl, CO-aryl, SO₂-aryl, CO-hetaryl or SO₂-alkylenearyl radical,
R_{E}¹⁷ is hydrogen, a hydroxyl group, CN, halogen, a branched or unbranched, optionally substituted C₁-C₆-alkyl radical, an optionally substituted C₃-C₇-cycloalkyl, aryl, hetaryl or arylalkyl radical, a C₂-C₆-alkynyl or C₂-C₆-alkenyl radical optionally substituted by C₁-C₄-alkyl or aryl, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocycle substituted by up to three identical or different radicals, which can contain up to three different or identical heteroatoms O, N, S, where two radicals together can be a fused, saturated, unsaturated or aromatic carbocycle or heterocycle which can contain up to three different or identical heteroatoms O, N, S and the cycle can optionally be substituted or a further, optionally substituted, saturated, unsaturated or aromatic cycle can be fused to this cycle, or the radical R_{E}¹⁷ forms, together with R_{W}² or R_{W}²*, a saturated or unsaturated C₃-C₇-heterocycle which can optionally contain up to two further heteroatoms selected from the group O, S and N,
R_{E}¹¹, R_{E}¹¹*
independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl, CO—NH—C₁-C₆-alkoxyalkyl, CO—NH—C₁-C₆-alkyl or SO₂—C₁-C₆-alkyl radical or an optionally substituted hetaryl, arylalkyl, C₃-C₇-cycloalkyl, CO—O-alkylenearyl, CO—NH-alkylenearyl, CO-alkylenearyl, CO-aryl, CO—NH-aryl, SO₂-aryl, CO-hetaryl, SO₂-alkylenearyl, SO₂-hetaryl or SO₂-alkylenehetaryl radical,
R_{E}¹² is hydrogen, a branched or unbranched, optionally substituted C₁-C₆-alkyl, C₂-C₆-alkenyl, C₂-C₈-alkynyl radical, an optionally substituted C₃-C₇-cycloalkyl, hetaryl, arylalkyl or hetarylalkyl radical or a radical CO—R_{E}¹⁶, COOR_{E}¹⁶ or SO₂—R_{E}¹⁶,
R_{E}¹³, R_{E}¹⁴
independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁵ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁶ is hydrogen, a hydroxyl group, a branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical,
with the proviso that in the case
where Y_{E} or Z_{E}=CO and a radical X_{E} or Q_{E} or an aromatic or heteroaromatic radical from the structural element A is bonded directly to Y_{E} or Z_{E}, a direct atomic bond from Y_{E} or Z_{E} to the structural element G is excluded,
A is a structural element selected from the group:
a 4- to 8-membered monocyclic saturated, unsaturated or aromatic hydrocarbon, which can contain up to 4 heteroatoms selected from the group O, N and S, where, in each case independently of one another, the ring nitrogen optionally present or the carbons can be substituted, with the proviso that at least one heteroatom selected from the group O, N and S is contained in the structural element A,
or
a 9- to 14-membered polycyclic saturated, unsaturated or aromatic hydrocarbon which can contain up to 6 heteroatoms selected from the group N, O and S, where, in each case independently of one another, the ring nitrogen optionally present or the carbons can be substituted, with the proviso that at least one heteroatom selected from the group O, N and S is contained in the structural element A,
a radical 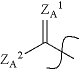
where
Z_{A}¹ is oxygen, sulfur or optionally substituted nitrogen and
Z_{A}² is optionally substituted nitrogen, oxygen or sulfur,
and a radical 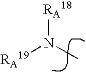
where
R_{A}¹⁸, R_{A}¹⁹
independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹¹,
and the physiologically tolerable salts, prodrugs and the enantiomerically pure or diastereomerically pure and tautomeric forms.

12. A compound as claimed in claim 11, wherein the structural element A used is a structural element selected from the group of structural elements of the formulae I_{A}¹ to I_{A}¹⁸ 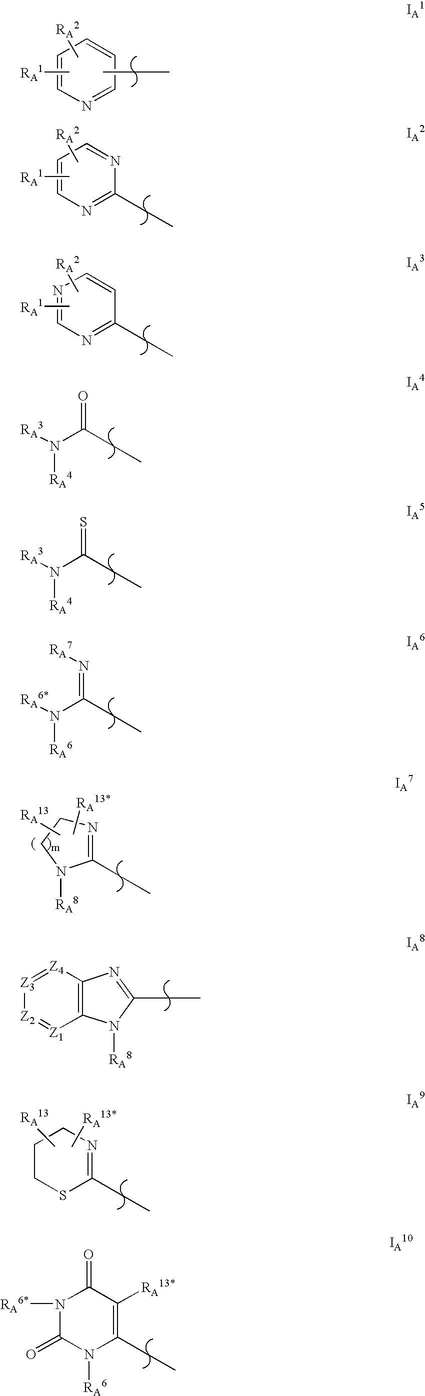 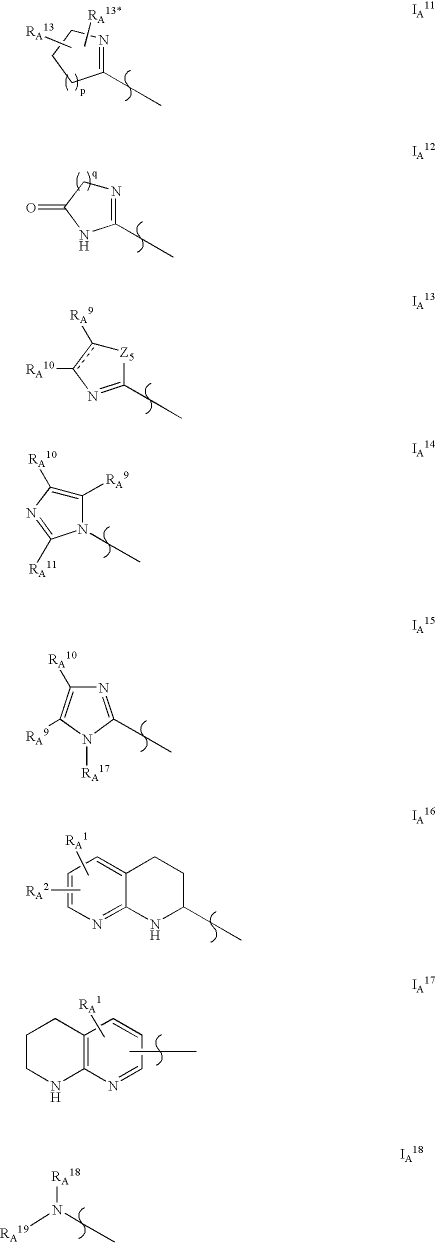
where
m, p, q
independently of one another are 1, 2 or 3,
R_{A}¹, R_{A}²
independently of one another are hydrogen, CN, halogen, a branched or unbranched, optionally substituted C₁-C₆-alkyl or CO—C₁-C₆-alkyl radical or an optionally substituted aryl, arylalkyl, hetaryl, hetarylalkyl or C₃-C₇-cycloalkyl radical or a radical CO—O—R_{A}¹⁴, O—R_{A}¹⁴, S—R_{A}¹⁴, NR_{A}¹⁵R_{A}¹⁶, CO—NR_{A}¹⁵R_{A}¹⁶ or SO₂NR_{A}¹⁵R_{A}¹⁶ or both radicals R_{A}¹ and R_{A}² together are a fused, optionally substituted, 5- or 6-membered, unsaturated or aromatic carbocycle or heterocycle which can contain up to three heteroatoms selected from the group O, N, and S,
R_{A}¹³, R_{A}¹³*
independently of one another are hydrogen, CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,
where
R_{A}¹⁴ is hydrogen, a branched or unbranched, optionally substituted C₁-C₆-alkyl, alkylene-C₁-C₄-alkoxy, C₂-C₆-alkenyl, C₂-C₆-alkynyl or C₁-C₆-alkylene-C₃-C₇-cycloalkyl radical or an optionally substituted C₃-C₇-cycloalkyl, aryl, arylalkyl, hetaryl or hetarylalkyl radical,
R_{A}¹⁵, R_{A}¹⁶,
independently of one another are hydrogen, a branched or unbranched, optionally substituted C₁-C₆-alkyl, CO—C₁-C₆-alkyl, SO₂—C₁-C₆-alkyl, COO—C₁-C₆-alkyl, CO—NH—C₁-C₆-alkyl, arylalkyl, COO-alkylenearyl, SO₂-alkylenearyl, CO—NH-alkylenearyl, CO—NH-alkylenehetaryl or hetarylalkyl radical or an optionally substituted C₃-C₇-cycloalkyl, aryl, CO-aryl, CO—NH-aryl, SO₂-aryl, hetaryl, CO—NH-hetaryl or CO-hetaryl radical,
R_{A}³, R_{A}⁴
independently of one another are hydrogen, —(CH₂)ₙ—(X_{A})ⱼ—R_{A}¹², or both radicals together are a 3- to 8-membered, saturated, unsaturated or aromatic N-heterocycle which can additionally contain two further, identical or different heteroatoms O, N or S, where the cycle is optionally substituted or a further, optionally substituted, saturated, unsaturated or aromatic cycle can be fused to this cycle,
where
n is 0, 1, 2 or 3,
j is 0 or 1,
X_{A} —CO—, —CO—N(R_{X}¹)—, —N(R_{X}¹)—CO—, —N(R_{X}¹)—CO—N(R_{X}¹*)—, —N(R_{X}¹)—CO—O—, —O—, —S—, —SO₂—, —SO₂—N(R_{X}¹)—, —SO₂—O—, —CO—O—, —O—CO—, —O—CO—N(R_{X}¹)—, —N(R_{X}¹)— or —N(R_{X}¹)—SO₂—,
R_{A}¹² is hydrogen, a branched or unbranched, optionally substituted C₁-C₆-alkyl radical, an optionally C₁-C₄-alkyl or aryl-substituted C₂-C₆-alkynyl or C₂-C₆-alkenyl radical or a 3- to 6-membered, saturated or unsaturated heterocycle, substituted by up to three identical or different radicals, which can contain up to three different or identical heteroatoms O, N, S, a C₃-C₇-cycloalkyl, aryl or hetaryl radical, where both radicals together can be a fused, saturated, unsaturated or aromatic carbocycle or heterocycle which can contain up to three different or identical heteroatoms O, N, S and the cycle can optionally be substituted or a further, optionally substituted, saturated, unsaturated or aromatic cycle can be fused to this cycle, or the radical R_{A}¹², together with R_{X}¹ or R_{X}¹* is a saturated or unsaturated C₃-C₇-heterocycle which can optionally contain up to two further heteroatoms selected from the group O, S and N,
R_{X}¹, R_{X}¹*
independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl or SO₂—C₁-C₆-alkyl radical or an optionally substituted C₃-C₇-cycloalkyl, aryl, arylalkyl, CO—O-alkylenearyl, CO-alkylenearyl, CO-aryl, SO₂-aryl, hetaryl, CO-hetaryl or SO₂-alkylenearyl radical,
R_{A}⁶, R_{A}⁶
are hydrogen, a branched or unbranched, optionally substituted C₁-C₄-alkyl, —CO—O—C₁-C₄-alkyl, arylalkyl, —CO—O-alkylenearyl, —CO—O-allyl, —CO—C₁-C₄-alkyl, —CO-alkylenearyl, C₃-C₇-cycloalkyl or —CO-allyl radical or in the structural element I_{A}⁷ both radicals R_{A}⁶ and R_{A}⁶* together are an optionally substituted, saturated, unsaturated or aromatic heterocycle which, in addition to the ring nitrogen, can contain up to two further different or identical heteroatoms O, N, S,
R_{A}⁷ is hydrogen, —OH, —CN, —CONH₂, a branched or unbranched, optionally substituted
C₁-C₄-alkyl, C₁-C₄-alkoxy, C₃-C₇-cycloalkyl or —O—CO—C₁-C₄-alkyl radical, or an optionally substituted arylalkyl, —O-alkylenearyl, —O—CO-aryl, —O—CO-alkylenearyl or —O—CO-allyl radical, or both radicals R_{A}⁶ and R_{A}⁷ together are an optionally substituted, unsaturated or aromatic heterocycle which, in addition to the ring nitrogen, can contain up to two further different or identical heteroatoms O, N, S,
R_{A}⁸ is hydrogen, a branched or unbranched, optionally substituted C₁-C₄-alkyl, CO—C₁-C₄-alkyl, SO₂—C₁-C₄-alkyl or CO—O—C₁-C₄-alkyl radical or an optionally substituted aryl, CO-aryl, SO₂-aryl, CO—O-aryl, CO-alkylenearyl, SO₂-alkylenearyl, CO—O-alkylenearyl or alkylenearyl radical,
R_{A}⁹, R_{A}¹⁰
independently of one another are hydrogen, —CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶, or both radicals R_{A}⁹ and R_{A}¹⁰ together in the structural element I_{A}¹⁴ are a 5- to 7-membered saturated, unsaturated or aromatic carbocycle or heterocycle which can contain up to three different or identical heteroatoms Q, N, S and is optionally substituted by up to three identical or different radicals,
R_{A}¹¹ is hydrogen, —CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,
R_{A}¹⁷ is hydrogen or, in the structural element I_{A}¹⁶, both radicals R_{A}⁹ and R_{A}¹⁷ together are a 5- to 7-membered saturated, unsaturated or aromatic heterocycle which, in addition to the ring nitrogen, can contain up to three different or identical heteroatoms O, N, S and is optionally substituted by up to three identical or different radicals,
R_{A}¹⁸, R_{A}¹⁹
independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bis-alkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R_{G}¹¹, —CO—OR_{G}¹¹, —CO—NR_{G}¹¹R_{G}¹¹* or —CO—R_{G}¹¹ which is independent of R_{G}¹¹,
Z¹, Z², Z³, Z⁴
independently of one another are nitrogen, C—H, C-halogen or a branched or unbranched, optionally substituted C—C₁-C₄-alkyl or C—C₁-C₄-alkoxy radical,
Z⁵ is NR_{A}⁸, oxygen or sulfur.

13. A compound as claimed in claim 11 or 12, wherein the structural element E′ used is a structural element of the formula I_{E1E2}
-E₂-E₁- I_{E1E2}
and E¹ and E² have the following meanings:
E¹ is a substructural element of the formula I_{E1}
—(Y_{E})ₖ₁—(CR_{E}¹R_{E}²)_{c}-(Q_{E})ₖ₂-(CR_{E}³R_{E}⁴)_{d}— I_{E1}
and
E² is a substructural element of the formula I_{E2}
(NR_{E}¹¹)ₖ₃—(CR_{E}⁵R_{E}⁶)_{f}-(Z_{E})ₖ₄-(CR_{E}⁷R_{E}⁸)_{g}—(X_{E})ₖ—(CR_{E}⁹R_{E}¹⁰)ₕ—(NR_{E}¹¹*)ₖ₆— I_{E2},
where
c, d, f, g, h
independently of one another are 0, 1 or 2,
k1, k2, k3, k4, k5, k6
independently of one another are 0 or 1,
X_{E}, Q_{E}
independently of one another are an optionally substituted 4- to 11-membered mono- or polycyclic, aliphatic or aromatic hydrocarbon, which can contain up to 6 double bonds and up to 6 identical or different heteroatoms selected from the group N, O, and S, where the ring carbons and/or the ring nitrogens can optionally be substituted,
Y_{E}, Z_{E}
independently of one another are CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴, C(═CR_{E}¹³R_{E}¹⁴), CR_{E}¹³═CR_{E}¹⁴, —CR_{E}¹³⁽OR_{E}¹⁵)—CHR_{E}¹⁴— or —CHR_{E}¹³—CR_{E}¹⁴⁽OR_{E}¹⁵)—,
R_{E}¹, R_{E}², R_{E}³, R_{E}⁴, R_{E}⁵, R_{E}⁶, R_{E}⁷, R_{E}⁸, R_{E}⁹, R_{E}¹⁰
independently of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)ₓ—(W_{E})_{z}—R_{E}¹⁷, an optionally substituted C₃-C₇-cycloalkyl, aryl, arylalkyl, hetaryl or hetarylalkyl radical, or
independently of one another in each case two radicals R_{E}¹ and R_{E}² or R_{E}³ and R_{E}⁴ or R_{E}⁵ and R_{E}⁶ or R_{E}⁷ and R_{E}⁸ or R_{E}⁹ and R_{E}¹⁰ together are a 3- to 7-membered, optionally substituted, saturated or unsaturated carbocycle or heterocycle which can contain up to three heteroatoms selected from the group-O, N and S,
x is 0, 1, 2, 3 or 4,
z is 0 or 1,
W_{E} is —CO—, —CO—N(R_{W}²)—, —N(R_{W}²)—CO—, —N(R_{W}²)—CO—N(R_{W}²*)—, —N(R_{W}²)—CO—O—, —O—, —S—, —SO₂—, —SO₂—N(R_{W}²)—, —SO₂—O—, —CO—O—, —O—CO—, —O—CO—N(R_{W}²)—, —N(R_{W}²)— or —N(R_{W}²)—SO₂—,
R_{W}², R_{W}²*
independently of one another are hydrogen, a branched or unbranched, optionally substituted C₁-C₆-alkyl, C₂-C₆-alkenyl, C₂-C₈-alkynyl, CO—C₁-C₆-alkyl CO—O—C₁-C₆-alkyl or SO₂—C₁-C₆-alkyl radical or an optionally substituted hetaryl, hetarylalkyl, arylalkyl, C₃-C₇-cycloalkyl, CO—O-alkylenearyl, CO-alkylenearyl, CO-aryl, SO₂-aryl, CO-hetaryl or SO₂-alkylenearyl radical,
R_{E}¹⁷ is hydrogen, a hydroxyl group, CN, halogen, a branched or unbranched, optionally substituted C₁-C₆-alkyl radical, an optionally substituted C₃-C₇-cycloalkyl, aryl, hetaryl or arylalkyl radical, a C₂-C₆-alkynyl or C₂-C₆-alkenyl radical optionally substituted by C₁-C₄-alkyl or aryl, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocycle substituted by up to three identical or different radicals, which can contain up to three different or identical heteroatoms O, N, S, where two radicals can together be a fused, saturated, unsaturated or aromatic carbocycle or heterocycle which can contain up to three different or identical heteroatoms O, N, S and the cycle can optionally be substituted or a further, optionally substituted, saturated, unsaturated or aromatic cycle can be fused to this cycle, or the radical R_{E}¹⁷ forms, together with R_{W}² or R_{W}²*, a saturated or unsaturated C₃-C₇-heterocycle which can optionally contain up to two further heteroatoms selected from the group O, S and N,
R_{E}¹¹, R_{E}¹¹*
independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl, CO—NH—C₁-C₆-alkoxyalkyl, CO—NH—C₁-C₆-alkyl or SO₂—C₁-C₆-alkyl radical or an optionally substituted hetaryl, arylalkyl, C₃-C₇-cycloalkyl, CO—O-alkylenearyl, CO—NH-alkylenearyl, CO-alkylenearyl, CO-aryl, CO—NH-aryl, SO₂-aryl, CO-hetaryl, SO₂-alkylenearyl, SO₂-hetaryl or SO₂-alkylenehetaryl radical,
R_{E}¹² is hydrogen, a branched or unbranched, optionally substituted C₁-C₆-alkyl, C₂-C₆-alkenyl, C₂-C₈-alkynyl radical, an optionally substituted C₃-C₇-cycloalkyl, hetaryl, arylalkyl or hetarylalkyl radical or a radical CO—R_{E}¹⁶, COOR_{E}¹⁶ or SO₂—R_{E}¹⁶,
R_{E}¹³, R_{E}¹⁴
independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁵ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁶ is hydrogen, a hydroxyl group, a branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical,
with the proviso that in the case where
Y_{E}═CO,
k1 and k5=1 and
h and k6=0
the sum of the indices c, k2 and d must be other than 0
and in the case where an aromatic or heteroaromatic radical from the structural element A is bonded directly to Y_{E} or Z_{E}, a direct atomic bond from Y_{E} or Z_{E} to the structural element G is excluded.

14. A compound as claimed in one of claims 11 to 13 for use as a drug.

15. The use of the compounds as claimed in one of claims 11 to 13 for the production of drugs for treating diseases.

16. A pharmaceutical preparation comprising, in addition to the customary pharmaceutical excipients, at least one compound as claimed in one of claims 11 to 13.

17. A pharmaceutical preparation, comprising at least one compound as claimed in one of claims 1 to 5, if appropriate pharmaceutical excipients and at least one further compound selected from the group
inhibitors of blood platelet adhesion, activation or aggregation,
anticoagulants which prevent thrombin activity or formation,
antagonists of blood platelet-activating compounds or selectin antagonists.

18. The use of the pharmaceutical preparation as claimed in claim 17 for the production of a drug for treating blood platelet-mediated vascular occlusion or thrombosis.

19. A pharmaceutical preparation, comprising at least one compound as claimed in one of claims 1 to 5, if appropriate pharmaceutical excipients and at least one further compound selected from the group
inhibitors of blood platelet activation or aggregation,
serine protease inhibitors,
fibrinogen-lowering compounds,
selectin antagonists,
antagonists of ICAM-1 or VCAM-1
inhibitors of leukocyte adhesion
inhibitors of vascular wall transmigration,
fibrinolysis-modulating compounds,
inhibitors of complement factors,
endothelin receptor antagonists,
tyrosine kinase inhibitors,
antioxidants and
interleukin 8 antagonists.

20. The use of the pharmaceutical preparation as claimed in claim 19 for the production of a drug for treating myocardial infarct or stroke.

21. A pharmaceutical preparation comprising at least one compound as claimed in one of claims 1 to 5, if appropriate pharmaceutical excipients and at least one further compound selected from the group
endothelin antagonists,
ACE inhibitors,
angiotensin receptor antagonists,
endopeptidase inhibitors,
beta-blockers,
calcium channel antagonists,
phosphodiesterase inhibitors and
caspase inhibitors.

22. The use of the pharmaceutical preparation as claimed in claim 21 for the production of a drug for treating congestive heart failure.

23. A pharmaceutical preparation comprising at least one compound as claimed in one of claims 1 to 5, if appropriate pharmaceutical excipients and at least one further compound selected from the group
thrombin inhibitors,
inhibitors of factor Xa,
inhibitors of the coagulation pathway which leads to thrombin formation,
inhibitors of blood platelet adhesion, activation or aggregation,
endothelin receptor antagonists,
nitrogen oxide synthase inhibitors,
CD44 antagonists,
selectin antagonists,
MCP-1 antagonists,
inhibitors of signal transduction in proliferating cells,
antagonists of the cell response mediated by EGF, PDGF, VEGF or bFGF and antioxidants.

24. The use of the pharmaceutical preparation as claimed in claim 23 for the production of a drug for treating restenosis after vascular injury or stent implantation.

25. A pharmaceutical preparation comprising at least one compound as claimed in one of claims 1 to 5, if appropriate pharmaceutical excipients and at least one further compound selected from the group
antagonists of the cell response mediated by EGF, PDGF, VEGF or bFGF,
heparin or low-molecular weight heparins or further GAGs,
inhibitors of MMPs,
selectin antagonists,
endothelin antagonists,
ACE inhibitors,
angiotensin receptor antagonists,
glycosylation inhibitors and
AGE formation inhibitors or AGE breakers and antagonists of their receptors.

26. The use of the pharmaceutical preparation as claimed in claim 25 for the production of a drug for treating diabetic angiopathies.

27. A pharmaceutical preparation comprising at least one compound as claimed in one of claims 1 to 5, if appropriate pharmaceutical excipients and at least one further compound selected from the group
lipid-lowering compounds,
selectin antagonists,
antagonists of ICAM-1 or VCAM-1
heparin or low-molecular weight heparins or further GAGs,
inhibitors of MMPs,
endothelin antagonists,
apolipoprotein A1 antagonists,
cholesterol antagonists,
HMG CoA reductase inhibitors,
ACAT inhibitors,
ACE inhibitors,
angiotensin receptor antagonists,
tyrosine kinase inhibitors,
protein kinase C inhibitors,
calcium channel antagonists,
LDL receptor function stimulants,
antioxidants
LCAT mimetics and
free radical scavengers.

28. The use of the pharmaceutical preparation as claimed in claim 27 for the production of a drug for treating atherosclerosis.

29. A pharmaceutical preparation comprising at least one compound as claimed in one of claims 1 to 5, if appropriate pharmaceutical excipients and at least one further compound selected from the group
cytostatic or antineoplastic compounds,
compounds which inhibit proliferation and
heparin or low-molecular weight heparins or further GAGs.

30. The use of the pharmaceutical preparation as claimed in claim 29 for the production of a drug for the treatment of cancer.

31. A pharmaceutical preparation comprising at least one compound as claimed in one of claims 1 to 5, if appropriate pharmaceutical excipients and at least one further compound selected from the group
compounds for antiresorptive therapy,
compounds for hormone exchange therapy,
recombinant human growth hormone,
bisphosphonates,
compounds for calcitonin therapy,
calcitonin stimulants,
calcium channel antagonists,
bone formation stimulants,
interleukin-6 antagonists and
Src tyrosine kinase inhibitors.

32. The use of the pharmaceutical preparation as claimed in claim 31 for the production of a drug for the treatment of osteoporosis.

33. A pharmaceutical preparation comprising at least one compound as claimed in one of claims 1 to 5, if appropriate pharmaceutical excipients and at least one further compound selected from the group
TNF antagonists,
antagonists of VLA-4 or VCAM-1,
antagonists of LFA-1, Mac-1 or ICAMs,
complement inhibitors,
immunosuppressants,
interleukin-1, -5 or -8 antagonists and
dihydrofolate reductase inhibitors.

34. The use of the pharmaceutical preparation as claimed in claim 33 for the production of a drug for treating rheumatoid arthritis.

35. A pharmaceutical preparation comprising at least one compound as claimed in one of claims 1 to 5, if appropriate pharmaceutical excipients and at least one further compound selected from the group
collagenase,
PDGF antagonists and
MMPs.

36. The use of the pharmaceutical preparation as claimed in claim 35 for the production of a drug for improving wound healing.
